(19) United States
(12) Patent Application Publication
(10) Pub. No.: US 2017/0209443 A1
(43) Pub. Date: Jul. 27, 2017

(54) ANTI-INFLAMMATORY EFFECT OF ORALLY ACTIVE FYN KINASE INHIBITOR SARA-CATINIB (AZD 0530) AGAINST PARKINSON'S DISEASE AND OTHER RELATED NEURODEGENARATIVE DISEASES

(71) Applicant: Iowa State University Research Foundation, Inc., Ames, IA (US)

(72) Inventors: Anumantha G. Kanthasamy, Ames, IA (US); Arthi Kanthasamy, Ames, IA (US)

(21) Appl. No.: 15/003,671

(22) Filed: Jan. 21, 2016

(51) Int. Cl. A61K 31/517; A61K 45/06; A61K 9/00

(52) U.S. Cl. CPC A61K 31/517; A61K 9/0053; A61K 45/06

(57) ABSTRACT

The present invention describes novel pharmaceutical compositions and methods for treatment of diseases, disorders, or conditions characterized by neuroinflammation, particularly, Parkinson's disease. According to the invention, compounds which inhibit the activity or expression of non-receptor Fyn tyrosine kinase can prevent activation of the neural inflammatory pathway and provide protection from and treatment of Parkinson's disease. Particularly preferred is the class of small molecule Fyn kinase inhibitors such as sacaratinib and its prodrugs, derivatives, analogs and the like.

### GRANT REFERENCE

This invention was made with government support under contract NIH Grant No. R01 NS088206. The Government has certain rights in this invention.

### FIELD OF THE INVENTION

This invention relates to methods and pharmaceutical compositions for inhibiting or protecting against neuroinflammation using a Fyn tyrosine kinase inhibitor. In particular, it relates to use of the small molecule Fyn inhibitor saracatinib in the treatment of diseases, disorders, and conditions where neuroinflammation is implicated. The invention also relates to methods of using Fyn tyrosine kinase inhibitors and pharmaceutical compositions of this invention to treat neurodegenerative diseases, disorders, and conditions, such as Parkinson's disease.

### BACKGROUND OF THE INVENTION

Parkinson's disease (PD) is a neurodegenerative disorder affecting more than 1% of the population over the age of 60 in the US (Allam et al., 2005; West et al., 2005). PD is characterized clinically by severe motor symptoms including uncontrollable resting tremor, muscular rigidity, impaired postural reflexes, and bradykinesia, which vary between patients. These abnormalities can be accompanied by other symptoms, such as autonomic dysfunction, depression, and a general slowing of intellectual processes. Pathologically, PD is characterized by the marked degeneration of dopaminergic neurons in the substantia nigra pars compacta, which leads to the depletion of dopamine (DA) in its striatal projections, and of other brainstem neurons, with consequent disruption of the cerebral neuronal systems responsible for motor functions. This neurodegeneration is accompanied by the presence of cytoplasmic (Lewy bodies, LBs) and neuritic (Lewy neurites, LNs) inclusions in the surviving dopaminergic neurons and other affected regions of the central nervous system (CNS), but the mechanism underlying their formation is unclear, as is their pathogenic relevance.

PD is an essentially sporadic neurodegenerative disease whose pathogenesis remains largely unknown, despite years of intense research in an attempt to explain the complexity and the relative selectivity of dopaminergic neurodegeneration.

There are nine members of Src family of intracellular non-receptor tyrosine kinases. Five of them (Src, Fyn, Lck, Lyn, and Yes) are expressed in the central nervous system, but Src and Fyn are most highly expressed in the brain. Fyn activity, like that of other Src family kinases, is regulated by intramolecular interactions that depend on an equilibrium between tyrosine phosphorylation and dephosphorylation (Thomas et al., 1997, Ann. Rev. Cell & Dev. Biol. 13:513-609). In the basal state, catalytic activity is constrained by intramolecular interactions, such as engagement of the SH2 domain by a phosphorylated C-terminal Tyr 527. Disruption of these interactions by phosphorylation at Tyr 416 in the activation loop of the kinase domain and/or by dephosphorylation of Tyr 527 results in Fyn activation (Hunter, 1987, Cell, 49: 1-4).

Fyn has been localized to the post-synaptic density (PSD) fraction of the brain and amongst its substrates are receptors for the major excitatory transmitter glutamate. Fyn regulates glutamate receptor trafficking and synaptic plasticity (Nakazawa et al., 2001, J Biol Chem 276:693-699; Kojima et al., 1998, Learning & Memory (Cold Spring Harbor N.Y.) 5:429-445; Grant et al., 1992, Science 258:1903-1910; Prybylowski et al., 2005, Neuron 47:845-857). Specifically, Fyn phosphorylates the NMDA-type glutamate receptor subunits, NR2A and NR2B (Suzuki et al., 1995, Biochem Biophys Res Commun 216:582-588).

Inhibitors of a kinase of the Src family, such as dasatinib, bosutinib, saracatinib or ponatinib, are used in the treatment of cancer, in particular lung cancer. Approximately 85% of all lung cancer incidences are non-small cell lung cancer (NSCLC) (Jemal, Siegel et al., 2008). Non-small cell lung cancer (NSCLC) is one of the two main types of lung carcinoma, non-small cell (80.4%) and small-cell (16.8%) lung carcinoma, the classification being based on histological criteria. The non-small cell lung carcinomas have a similar prognosis and similar management and comprise three sub-types: squamous cell lung carcinoma, adenocarcinoma and large cell lung carcinoma. Squamous cell lung carcinoma (31.1% of lung cancers) often starts near a central bronchus and commonly shows cavitation and necrosis within the center of the cancer. Adenocarcinoma (29.4% of lung cancers) mostly originates in peripheral lung tissue and is usually associated with smoking. Large cell lung carcinoma (10.7% of lung cancers) is a fast-growing form that develops near the surface of the lung. Common treatments of NSCLC include surgery, chemotherapy, and radiation therapy. In particular, NSCLC is treated with adjuvant chemotherapy (i.e. chemotherapy after surgery). Wu (2009) describes phosphorylation patterns in lung cancer (see Wu (2009) PloS ONE 4 (11) e7994).

It is an object of the current invention to provide novel pharmaceutical compositions and treatment protocols for Parkinson's disease and associated neuroinflammation through inhibition of Src intracellular non-receptor tyrosine kinases. Preferably treatment of Parkinson's disease without dementia, and more preferably treatment of Parkinson's without concomitant Amyloid-β accumulation.

### BRIEF SUMMARY OF THE INVENTION

The present invention provides methods and a pharmaceutical compositions comprising an effective, orally active amount of the Fyn tyrosine kinase inhibitor, for use in treating of Parkinson's and other related neurodegenerative disease.

The present invention further provides a method of treating or preventing inflammation in Parkinson's disease or other neurological disorder in a mammal in need thereof, the method comprising administering to the mammal a therapeutically effective amount of a Fyn inhibitor. The present invention further provides a method of preventing further neurodegeneration in a mammal in need thereof, the method comprising administering to the mammal a therapeutically effective amount of a Fyn inhibitor.

In certain embodiments, the Fyn inhibitor is selected from the group consisting of a nucleic acid, siRNA, antisense nucleic acid, ribozyme, peptide, antibody, small molecule, antagonist, aptamer, peptidomimetic, and any combinations thereof. In preferred embodiments, the Fyn small molecule inhibitor is selected from the group consisting of saracatinib, bosutinib, dasatinib, ponatinib, PP2, a salt, prodrug or solvate thereof, a derivative thereof, and any combinations thereof.

In certain embodiments, the composition is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes. In a preferred embodiment administration is oral.

In certain embodiments, the method further comprises administering to the mammal at least one additional agent that treats or prevents the Parkinson's or other neurological disease states, or other Fyn tyrosine kinase modulated diseases or disorders in the mammal. In yet other embodiments, the method further comprises administering to the mammal at least one additional agent that improves or prevents further neurodegeneration in the mammal. In yet other embodiments, the composition and at least one additional agent are co-formulated.

The present invention further provides a kit for preventing or treating an inflammation associated with Parkinson's disease or other neurological in a mammal, wherein the kit comprises a pharmaceutical composition of the invention, an applicator, and an instructional material for use thereof. In certain embodiments, the instructional material recites the amount of, and frequency with which, the composition is to be administered to the mammal.

In the preceding aspects of the invention, inhibitors of Fyn tyrosine kinase can be included in pharmaceutical compositions and administered to a mammal suffering from or at risk of a disease, disorder, or condition associated with Fyn tyrosine kinase activation, including but not limited to Parkinson's disease, Alzheimer's disease, Tourette's syndrome, schizophrenia, Huntington's disease, symptoms of attention deficit hyperactivity disorder, drug abuse and clinical depression. In a still further aspect, the invention concerns a method for treating neurodegeneration of neurons in a mammal in need thereof comprising administering to a mammal in need thereof a Fyn tyrosine kinase inhibitor, thereby providing neuroprotection. In another aspect, the pharmaceutical composition may be used in the treatment of diseases, disorders, or conditions associated with neurodegeneration. In one aspect, the neuron is a sympathetic, parasympathetic, or enteric, e.g. dorsal root ganglia neurons, motor neurons, and central neurons, e.g. neurons from the spinal cord.

### BRIEF DESCRIPTION OF THE DRAWINGS

FIGS. 1A-1E shows that Fyn and PKCδ are differentially expressed in primary astrocytes and microglia. FIG. 1A is a representative image from immunocytochemical analysis for the microglial marker Iba-1 and the astrocytic marker GFAP on both, the magnetically purified and pour-off fractions of cells obtained post-separation revealed almost no astrocytic contamination in the samples. Scale bar, 200 microns. FIG. 1B shows the colocalization image of Hoechst (nuclear stain) and Iba-1 images in 6 random image fields were obtained using the ImageJ plugin JACoP. FIG. 1C shows the number of Hoechst-positive and colocalization-positive cells were counted using ImageJ. The magnetically purified samples were >97% positive for microglial cells. FIGS. 1D and 1E, show western Blot analyses of the magnetically separated cells revealed that the microglial fraction expressed higher amounts of the non-receptor Src kinase Fyn and the serine threonine kinase PKCδ than did the astrocyte-rich pour-off fraction (*p<0.05, **p<0.01).

FIGS. 2A-2I show Fyn kinase is rapidly activated in microglial cells and in the ventral midbrain following inflammogen stimulation. FIG. 2A shows a Fyn kinase assay shows that Fyn activity was highly induced in BV2 microglia treated with 1 μg/mL LPS for 10, 15 and 30 min. (*p<0.05, **p<0.01). FIG. 2B are immunoblots showing a concomitant rise in p-Y416 SFK levels in BV2 cell lysates post-LPS treatment. FIGS. 2C and 2D show immunoprecipitation studies revealed that Fyn+/+ (WT Fyn), but not active loop tyrosine-mutant Fyn (Y417A Fyn), when overexpressed in BV2 microglia, was activated following LPS stimulation. FIG. 2E shows treatment of primary microglia with LPS. FIG. 2F shows TNFα treatment for 15 and 30 min increased p-Y416 SFK levels in primary microglia obtained from wild-type Fyn+/+, but not Fyn−/− mice, identifying Fyn as the primary Src family kinase that was activated by inflammogen stimulation. FIG. 2G shows pretreatment of primary microglia with the TLR-signaling antagonist IAXO-101 or the TNFα receptor decoy Etanercept abolished Fyn activation by LPS or TNFα stimulation (p-44/42 phosphorylation used as marker for early microglial activation) FIG. 2H shows immunocytochemistry of LPS-treated WT primary microglia showing that activated Fyn expression greatly increased and was localized preferentially to the membrane periphery of the microglial cell. Scale bar, 20 microns. FIG. 2I shows immunoblots of ventral midbrain lysates showed that peripheral administration of the inflammogen LPS (5 mg/kg) increased p-Y416 SFK levels in Fyn+/+, but not in Fyn−/− ventral midbrain tissues.

FIGS. 3A-3G show Fyn contributes to LPS- and TNFα-induced tyrosine phosphorylation and activation of PKCδ in primary microglia. Western blot analysis revealed that stimulation of microglia with LPS (FIG. 3A, 3B) and TNFα (FIG. 3C, 3D) induced a time-dependent increase in p-Y311 PKCδ levels in wild type but not Fyn−/− microglia (*p<0.05, **p<0.01). FIG. 3E shows LPS-induced PKCδ kinase activity was reduced in Fyn−/− microglial lysates in contrast to wild type lysates, as measured by PKCδ kinase assay. FIGS. 3F and 3G show co-immunoprecipitation studies showed that LPS stimulation elicited a physical interaction between Fyn and PKCδ in WT Fyn-transfected BV2 microglial cells.

FIGS. 4A-4D show the Fyn-PKCδ signaling axis mediates MAP kinase activation in microglial cells. FIGS. 4A and 4B show immunoblot analysis demonstrated diminished LPS-induced p38 and p44/42 (p-ERK) phosphorylation in Fyn−/− and PKCδ−/− microglia (*p<0.05, ***p<0.001). FIGS. 4C and 4D show diminished TNFα-induced p38 and p44/42 (p-ERK) phosphorylation in Fyn−/− and PKCδ−/− microglia (*p<0.05).

FIGS. 5A-5E show Fyn contributes to inflammogen-mediated NFκB pathway activation in microglial cells. FIGS. 5A and 5B show immunoblot analyses of whole cell lysates of wild-type and Fyn−/− microglia treated with LPS for 15-45 min revealed reduced IκBα degradation in Fyn−/− microglia at 15 min, and attenuated IκBα resynthesis at 30 and 45 min (*p<0.05, **p<0.01). FIGS. 5C and 5D show cytosolic and nuclear fractionation of LPS- and TNFα-treated wild type and Fyn−/− microglia revealed diminished nuclear translocation of the p65 subunit of the NFκB complex in the Fyn−/− microglia (**p<0.01, ***p<0.001). FIG. 5E is immunocytochemistry showing reduced nuclear p65 in LPS-treated Fyn−/− microglia. Scale bar, 50 microns.

FIGS. 6A-6G shows LPS- or TNFα-induced proinflammatory cytokine production is suppressed in Fyn/PKCδ deficient microglia. FIG. 6A shows Luminex analyses of supernatants from LPS-treated wild-type, PKCδ−/− and Fyn−/− microglia revealed reduced secretion of the pro-inflammatory cytokines IL-6, IL-12 and TNFα (**p<0.01, ***p<0.001). FIG. 6B shows wild-type primary microglia were transfected with non-targeting and Fyn-specific siRNA for 72 hours. Knockdown of Fyn was evaluated by Western blot. FIG. 6C shows Fyn depleted microglia demonstrated diminished IL-6 and TNFα secretion in response to LPS stimulation (**p<0.01, ***p<0.001). FIG. 6D shows TNFα stimulation of Fyn−/− microglia reduced IL-6 and TNFα production in contrast to wild-type microglia. (**p<0.01, ***p<0.001). FIG. 6E shows immunoblots showing reduced TNFα levels in Fyn-deficient microglia after TNFα stimulation in contrast to wild-type microglia. FIGS. 6F and 6G show overexpressing the FLAG-tagged activation loop tyrosine mutant of Fyn in BV2 microglia attenuated IL-6 and IL-12 production when the cells were treated with LPS, as shown by Luminex cytokine analysis (*p<0.05, **p<0.01 and ***p<0.001).

FIGS. 7A-7F show Fyn plays a role in LPS-induced iNOS expression, nitrite production and neuroinflammatory marker expression. FIG. 7A shows Griess nitrite measurement assay demonstrated that LPS-induced nitrite production was reduced in Fyn−/− microglia (*p<0.05, ***p<0.001). FIGS. 7B, 7C, and 7D show diminished iNOS expression in LPS-treated Fyn−/− microglia (**p<0.01). Scale bar, 100 microns. FIGS. 7E and 7F show reduced gp91phox and Iba-1 expression in LPS-treated Fyn−/− and PKCδ−/− microglia, as shown by immunoblotting analysis (*p<0.05, **p<0.01).

FIGS. 8A-8F show Fyn^{-/-} and PKCδ^{-/-} mice are resistant to LPS- and MPTP-induced neuroinflammatory responses. FIG. 8A show wild-type, PKCδ^{-/-} and Fyn^{-/-} mice were injected intraperitoneally with 5 mg/kg LPS for 3 h. Striatal cytokine mRNA levels, assessed by q-RT PCR, showed significantly reduced induction of pro-IL-1β and TNFα mRNA levels in PKCδ^{-/-} and Fyn^{-/-} mice in contrast to wild-type mice (*p<0.05, **p<0.01 and ***p<0.001). FIG. 8B shows the transitional stages of microglial activation, from ramified (inactivated, type A) to amoeboid (activated, types B, C and D), are shown by representative images. FIGS. 8C, and 8D show Iba-1-DAB immunohistochemistry in MPTP-injected Fyn^{-/-} and wild-type ventral midbrain sections demonstrated nigral microgliosis, assessed by quantification of microglial morphology, in the wild-type, but not the Fyn^{-/-} sections. Scale bar, 75 microns (*p<0.05, **p<0.01). FIGS. 8E, and 8F show Fyn^{-/-} mice showed diminished induction of the proinflammatory marker gp91^{phox} in ventral midbrain lysates following the acute MPTP regimen (*p<0.05).

FIGS. 9A-9D show Fyn−/− mice are protected against 6-OHDA-induced nigrostriatal dopaminergic neuronal deficits and microgliosis. FIG. 9A shows TH-DAB immunohistochemistry in 6-OHDA-injected Fyn−/− and wild-type mouse striatal sections. Scale bar, 1000 microns. FIG. 9B is a schematic diagram of a coronal section through the mouse striatum at the level of the injection. FIG. 9C shows significant preservation of 6-OHDA-induced degeneration of dopaminergic terminals is seen in the Fyn−/− mice in contrast to wild-type mice (**p<0.01, ***p<0.001). FIG. 9D shows immunofluorescence staining of 6-OHDA-injected Fyn−/− and wild-type ventral midbrain sections reveals diminished microgliosis and concomitant nigral neuroprotection in Fyn−/− mice after 6-OHDA administration, in contrast to the massive microgliosis and nigral dopaminergic neuronal death observed in the wild-type mice. Scale bar, 200 microns.

FIGS. 10A-10E show PKCδ^{-/-} mice are resistant to 6-OHDA-induced nigrostriatal dopaminergic neuronal deficits and microgliosis. FIG. 10A shows TH-DAB immunohistochemistry in 6-OHDA-injected PKCδ^{-/-} and wild-type mouse striatal sections. Scale bar, 1000 microns. FIG. 10B is a schematic diagram of a coronal section through the mouse striatum at the level of the injection. FIG. 10C shows significant preservation of dopaminergic terminals is seen in the 6-OHDA-treated PKCδ^{-/-} mice in contrast to wild-type mice (*p<0.05, **p<0.01 and ***p<0.001). FIG. 10D shows immunofluorescence staining of 6-OHDA-injected PKCδ^{-/-} and wild-type ventral midbrain sections reveals reduced nigral TH degeneration and microgliosis in PKCδ^{-/-} mice after 6-OHDA administration, in contrast to the wild-type mice. Scale bar, 200 microns. FIG. 10E shows a high magnification image of 6-OHDA-injected PKCδ^{-/-} and wild-type ventral midbrain sections. Scale bar, 50 microns.

FIGS. 11A-11F show diminished 6-OHDA-induced glial-neuronal contact (gliapse) formation in the Fyn^{-/-} substantia nigra. FIGS. 11A and 11C show confocal Z stack maximum projection image analysis of ventral midbrain sections reveals a strongly increased number of microglial-neuronal contacts and appositions upon 6-OHDA treatment of Fyn^{+/+} but not Fyn^{-/-} mice. Scale bar, 12 microns FIGS. 11B and 11D show confocal Z stack images were rotated and optically sectioned along the Z plane using Imaris software, allowing easy visualization of gliapse formation. Scale bar, 10 microns. FIG. 11E shows diagrams of Process-Body (Pr-B) and Body-Body (B-B) gliapses formed between dopaminergic neurons and microglia. FIG. 11F shows Fyn ventral midbrain sections revealed significantly fewer gliapses formed per dopaminergic neuron in the SN (***p<0.001).

FIGS. 12A-12F show prolonged inflammogen stimulation induces Fyn upon microglial activation. FIGS. 12A and 12B show stimulation of primary microglia with LPS for 12 h and TNFα for 24 h increased Fyn expression, as evidenced by Western blotting (*p<0.05). FIG. 12C shows ICC analysis of Fyn expression. Scale bar, 20 microns. FIG. 12D shows q-RT PCR analysis of Fyn mRNA levels in LPS-stimulated primary microglia and BV2 microglia revealed induction of Fyn at the message level (*p<0.05, **p<0.01). FIG. 12E shows the induction of Fyn promoter activity in primary microglia following LPS activation of wild-type primary microglia (*p<0.05). FIG. 12F shows increased striatal Fyn mRNA levels were seen in the Fyn^{+/+} mice injected intraperitoneally with LPS (5 mg/kg) for 12 h, as assessed by q-RT PCR (**p<0.01).

FIG. 13 shows a proposed scheme of Fyn-mediated neuroinflammatory signaling pathway in microglia. LPS and TNFα bind to their receptors, TLR4 and TNFR1 respectively, leading to early Fyn activation. Fyn then phosphorylates and activates PKCδ, which leads to the downstream activation of the MAP kinase and NFκB pathways. The p65 component of the NFκB complex enters the nucleus and binds to the promoter of various proinflammatory cytokine genes. Fyn is also upregulated to sustain the heightened inflammatory response during prolonged stimulation of microglia, possibly contributing to progressive neurodegeneration in PD.

FIGS. 14A-14I show aggregated α-synuclein acts as a danger signal to elicit NLRP3 inflammasome dependent IL-1β processing in LPS primed microglia. FIG. 14A shows aggregated αSyn was able to elicit significant IL-1β, but not TNFα production as early as 4 hours post stimulation in LPS primed microglia. Pretreatment of the cells with pan-caspase and caspase-1 specific inhibitors post priming but before αSyn treatment strongly attenuated the production of IL-1β, but minimally affected TNFα production (^{ns}p>0.05, *p<0.05, ***p<0.001). FIG. 14B shows the αSyn mediated production of IL-1β was largely attributable to the NLRP3 inflammasome, Supernatant cytokine analysis from LPS primed, αSyn treated WT, NLRP3−/−, ASC−/−, Caspase-1−/− and Caspase-11−/− BMDMs revealed strongly diminished IL-1β, but not TNFα production from the NLRP3−/−, ASC−/− and Caspase 1−/−, but minimally affected IL-1β production from the Caspase-11−/− cells, indicating that the canonical activation of the NLRP3 inflammasome was primarily responsible for the IL-1β production in response to aggregated αSyn (^{ns}p>0.05, *:p<0.05, ***:p<0.001). FIGS. 14C and 14D show no discernable changes in the LPS induced pro-IL-1β levels in WT, NLRP3−/−, ASC−/−, Caspase-1−/− and Caspase-11−/− BMDMs (^{ns}p>0.05) FIGS. 14E and 14F show aggregated αSyn treatment induced speck formation in primary microglia (*p<0.05) FIG. 14G shows aggregated αSyn treatment induced speck formation in the ASC-CFP reporter cell line. FIG. 14H shows immunoblot analysis of 4 month old A53T striatal lysates revealed significant increase in the levels of cleaved Caspase-1 levels, when compared to littermate controls (***p<0.001) FIG. 14I shows immunoblot analysis of PD nigral tissue lysates revealed significantly increased IL-1β and Caspase-1 p20 levels when compared to age matched control nigral lysates.

FIG. 15 shows the strategy for obtaining and purifying human alpha-synuclein.

FIG. 16 shows microglial Fyn is upregulated in PD patient brains over age-matched control brains. A, PD patient and age-matched control ventral midbrain sections were stained for Iba-1 and Fyn. PD patient brains display more Iba-1 expression, and increased Fyn expression within the Iba-1, indicating microgliosis and microglial Fyn upregulation.

FIGS. 17A-17B show Fyn kinase contributes to the priming of the NLRP3 inflammasome in response to diverse inflammogens. FIG. 17A shows WT and Fyn^{-/-} microglia were treated with various doses of LPS (10. 100 and 1000 ng/mL) and TNFα (10 and 30 ng/mL). Both inflammogens elicited a dose-dependent induction of pro-IL-1β and NLRP3 in WT microglia, but did so to a significantly lesser extent in the Fyn^{-/-} microglia. FIG. 17B shows Fyn^{-/-} mice treated with LPS (5 mg/kg) for 24 hours showed diminished serum secretion of IL-1β when compared to Fyn^{+/+} mice (**p<0.01).

FIGS. 18A-18G show aggregated α-synuclein amplifies LPS induced priming of the NLRP3 inflammasome and induces Caspase-1 activation, and pro-inflammatory cytokine and nitrite release in a Fyn dependent manner. FIG. 18A shows aggregated αSyn potentiated the LPS mediated induction of pro-IL-1β and NLRP3, but did so to a lesser extent in the Fyn deficient microglia (*p<0.05, **p<0.01, ***p<0.001). FIGS. 18B and 18C show treatment of microglial cells post priming and pre αSyn treatment with the NF-κB inhibitor SN-50 prevented the induction of pro-IL-1β and NLRP3 proteins (*p<0.05,**p<0.01). FIG. 18D shows αSyn treatment also increased the induction of pro-IL-1β and NLRP3 mRNAs in LPS treated WT, but not Fyn^{-/-} microglial cells (^{ns}p>0.05, *p<0.05). FIG. 18E shows the FLICA assay revealed strongly increased Caspase-1 activation in αSyn treated WT but not Fyn^{-/-} microglia (^{ns}p>0.05, *p<0.05). FIG. 18F shows LPS primed αSyn treated WT microglia produced higher amounts of pro-inflammatory cytokines than Fyn deficient microglia (*p<0.05, ***p<0.001). FIG. 18G shows αSyn treatment induced the production of supernatant nitrite and NOS2 induction, but did so to a significantly lesser extent in the Fyn deficient microglia (***p<0.001).

FIGS. 19A-19E show aggregated α-synuclein can prime and activate the NLRP3 inflammasome to mediate IL-1β production. FIG. 19A shows aggregated αSyn elicited Caspase-1 independent induction of pro-IL-1β and NLRP3 levels, as evidenced by immunoblot analysis from αSyn treated WT and Caspase-1^{-/-} microglial cell lysates. FIG. 18B shows αSyn treatment induced the Caspase-1 dependent production of IL-1β from microglial cells, which was also inhibited with pretreatment of the Fyn inhibitor Saracatinib in a dose dependent manner (^{ns}p>0.05, ***:p<0.001). FIG. 19C shows NLRP3^{-/-}, ASC^{-/-} and Caspase-1^{-/-} BMDMs demonstrated equable αSyn induced induction of pro-IL-1β and uptake of αSyn, but almost completely attenuated Caspase-1 cleavage. FIG. 18D shows αSyn induced Caspase-1p20 secretion in WT, but not NLRP3^{-/-} and Caspase-1^{-/-} supernatants (^{ns}p>0.05,*p<0.05, ***p<0.001), FIG. 18E shows the aggregated αSyn mediated production of IL-1β, but not TNFα was strongly attenuated in NLRP3^{-/-}, ASC^{-/-}, and Caspase 1^{-/-}, but minimally affected in Caspase-11^{-/-} BMDM supernatants, indicating that αSyn is able to both prime, as well as activate the NLRP3 inflammasome primarily through its canonical activation.

FIGS. 20A-20D show CD36 associated Fyn is rapidly activated following α-synuclein stimulation, following which it tyrosine phosphorylates PKCδ. FIG. 20A shows upon its treatment to microglial cells, aggregated αSyn associates with TLR-2 and CD36, as evidenced by co-immunoprecipitation analysis. Upon αSyn treatment, Fyn associates with CD36, but not TLR2. FIG. 20B shows immunoblot analysis of aggregated αSyn treated WT and Fyn^{-/-} microglial lysates reveals a rapid induction of Src family kinase activation in WT, but not Fyn^{-/-} microglia. FIG. 20C shows whole cell lysate analysis from αSyn treated wild type (WT) and activation loop deficient (Y417A) Fyn-FLAG transfected cells revealed a perfect overlap between the p-SFK Y416 and FLAG bands in the 30 and 45 min αSyn treated WT Fyn FLAG transfected samples, and a complete absence of p-SFK Y416 activation in the Fyn activation loop mutant transfected samples. FIG. 20D shows immunocytochemistry analysis reveals a rapid increase in p-SFK Y416 levels in αSyn treated Iba-1 positive WT microglial cells.

FIGS. 21A-21I show Fyn contributes to α-synuclein mediated priming of the NLRP3 inflammasome, resulting in diminished IL-1β and other pro-inflammatory cytokine production. FIGS. 21A and 21B show diminished αSyn induced nuclear translocation of NF-κB-p65 in the Fyn^{-/-} microglial cells (**p<0.01) FIG. 21C show diminished induction of pro-IL-1β and NLRP3 mRNA levels in the Fyn deficient microglial upon αSyn treatment (^{ns}p>0.05, **p<0.01, ***p<0.001). FIGS. 21D and 21 E show reduced induction of pro-IL-1β and NLRP3 protein levels, as well as Caspase-1 and IL-1β cleavage in Fyn^{-/-} microglia (^{ns}p>0.05, *p<0.05, **p<0.01, ***p<0.001). FIGS. 21F and 21G show knocking down Fyn using si-RNA reduces the αSyn mediated induction of pro-IL-1β in primary WT microglia (*p<0.05). FIGS. 21H and 21I show reduced supernatant IL-1β and other pro-inflammatory cytokine production from αSyn treated Fyn^{-/-} microglia (***p<0.001).

FIGS. 22A-22G show Fyn contributes to aggregated α-synuclein uptake into microglial cells, resulting in the mitochondrial ROS generation. FIGS. 22A and 22B show immunocytochemistry (for human α-synuclein) revealed diminished uptake of the protein in the Fyn deficient microglia (*p<0.05). FIGS. 22C and 22D show immunoblot analysis also reveals that human α-synuclein is taken up at lesser levels into Fyn−/− microglia (*p<0.05). FIG. 22E shows diminished mitoROS generation from αSyn treated Fyn−/− microglia (**p<0.01). FIGS. 22F and 22G shows diminished mitochondrial morphology deficits observed in the aggregated αSyn treated Fyn−/− microglia.

FIGS. 23A-23G show α-synuclein treatment brings about Fyn mediated PKCδ activation, which contributes to aggregated α-synuclein mediated priming of the NLRP3 inflammasome, but not to α-synuclein import into microglia. FIG. 23A shows immunoblot analysis of aggregated α-synuclein treated WT and Fyn^{-/-} microglial lysates reveals a rapid induction of pY311-PKCδ levels in the WT, but not Fyn^{-/-} microglia. FIGS. 23B and 23C show reduced aggregated α-synuclein mediated p65 nuclear translocation seen in PKCδ^{-/-} microglia (**p<0.01). FIG. 23D shows attenuated α-synuclein induced pro-IL-1β mRNA induction in PKCδ deficient microglia (***p<0.001) FIG. 23E shows reduced induction of pro-IL-1β and NLRP3 proteins, and FIG. 23F, secretion of IL-1β from PKCδ^{-/-} microglia. FIG. 23G shows no change in the import of aggregated α-synuclein import observed between PKCδ^{+/+} and PKCδ^{-/-} microglia (***p<0.001).

FIG. 24 shows aggregated α-synuclein mediated NLRP3 inflammasome activation pathway Aggregated α-synuclein binds to the receptors TLR-2 and CD36 on microglial cells. CD36 recruits Fyn kinase, which in turn is activated and tyrosine phosphorylates PKCδ at Y311, leading to increased PKCδ dependent activation of the NF-κB pathway. p65 translocates to the nucleus and brings about the induction of pro-IL-1β and NLRP3 mRNAs. Aggregated α-synuclein is also taken up by the microglia, following which it brings about mitochondrial dysfunction mediated activation of the NLRP3 inflammasome. Fyn, but not PKCδ contributes to this process as well.

FIGS. 25A-25F show diminished microgliosis, inflammasome activation in Fyn deficient mice using the AAV-SYN PD model. FIG. 25A shows stereotactic injection of AAV-GFP and AAV-SYN overexpressing particles into the SNpc resulted in a specific targeting of the SNpc dopaminergic neurons, as indicated by a co-localization of TH and GFP/human αSyn expression. FIG. 25B shows massive microgliosis was observed within the SN in the Fyn^{+/+} mice injected with the AAV-SYN particles, but not the Fyn^{-/-} mice. FIG. 25C shows a 3-D reconstruction of the Z-stack images in the ventral midbrain of AAV-SYN injected WT and Fyn^{-/-} reveals the disparity of the microglial response/neuron between the genotypes. FIG. 25D shows a representational diagram of AAV injection site in the mice. FIG. 25E shows the formation of ASC specks within the microglial cells in the Fyn^{+/+}, but not Fyn^{-/-} AAV-SYN injected ventral midbrain sections. FIG. 25F shows quantification of the midbrain ASC specks upon AAV-GFP or AAV-SYN injection in Fyn^{+/+} and Fyn^{-/-} ventral midbrain sections (^{ns}p>0.05, *p<0.05).

FIGS. 26A-26C show Saracatinib, a Src family kinase inhibitor, attenuates aggregated α-synuclien induced inflammation in microglial cell line. FIG. 26A shows Saracatinib treatments at low dose was not associated with cell death at low dose. FIG. 26B shows Saracatinib attenuates the nitrite production induced by aggregated α-synuclein in microglia at 10 μM dose. FIG. 26C shows Saracatinib attenuates the secretion of pro-inflammatory cytokines IL-1β, IL-12 and TNF-α induced by aggregated α-synuclein in microglial cell line (***p<0.001, **p<01, *p<0.05 and ^{ns}p>0.05).

FIG. 27 shows Saracatinib treatment blocked activation of microglia in the substantia nigra of MPTP-treated mice. Control mice received saline. The substantia nigra (SN) tissue sections were immunostained with antibodies against IBA-1. Representative Western blot illustrating increased expression of IBA-1 in MPTP-treated animals but not in Saracatinib and MPTP-treated animals.

### DETAILED DESCRIPTION OF THE INVENTION

### Definitions

As used herein, the term “Fyn tyrosine kinase inhibitor” includes any compound capable of downregulating, decreasing, reducing, suppressing or inactivating the amount and/or activity of Fyn tyrosine kinase. Generally, said inhibitors may be proteins, oligo- and polypeptides, polynucleotides, genes, lipid, polysaccharide, drugs, small chemical molecules, or other chemical moieties. Inhibitors for use with the invention may function to inhibit Fyn tyrosine kinase by any number of ways, including decreasing Fyn tyrosine kinase mRNA or protein levels or by blocking the activation of Fyn tyrosine kinase or its activity, for example, through inhibiting or decreasing proteolytic cleavage of Fyn tyrosine kinase using a Fyn tyrosine kinase peptide cleavage inhibitor. Compounds that decrease activity of Fyn tyrosine kinase downstream of Fyn tyrosine kinase in its pathway and decrease products or activity of Fyn tyrosine kinase targets, for example, PP2A and TH, or decrease activity upstream of Fyn tyrosine kinase are also within the scope of Fyn tyrosine kinase inhibitors of the present invention.

As used herein, the term “neurodegeneration” refers the damage or death of a cell in the central nervous system, for example, a neuron. Neurodegeneration refers to any pathological changes in neuronal cells, including, without limitation, death or loss of neuronal cells and any changes that precede cell death. The pathological changes may be spontaneous or may be induced by any event and include, for example, pathological changes associated with apoptosis. The neurons may be any neurons, including without limitation sensory, sympathetic, parasympathetic, or enteric, e.g. dorsal root ganglia neurons, motor neurons, and central neurons, e.g. neurons from the spinal cord.

As used herein, the terms “neurodegenerative disorder” or “neurodegenerative disease” refer broadly to disorders or diseases that affect the nervous system having damage or death of a cell of the central nervous system, including but not limited to Parkinson's disease, Alzheimer's disease, Huntington's disease and amyotrophic lateral sclerosis.

As used herein, the term “compound” refers to a polynucleotide, a protein, a polypeptide, a peptide, an antibody, an immunoglobulin, a ligand, a cytokine, a growth factor, a nucleic acid, a lipid, membrane, a carbohydrate, a drug, a prodrug, or a small molecule or a fragment thereof.

As used herein, the term “modulates” refers to the ability of a compound to alter the mRNA or protein expression level or kinase activity or phosphatase activity of a protein.

As used herein, the term “pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition.

As used herein, the terms “pharmaceutically effective” or “therapeutically effective” shall mean an amount of a Fyn tyrosine kinase inhibitor that is sufficient to show a meaningful patient benefit, i.e., treatment, prevention, amelioration, or a decrease in the frequency of the condition or symptom being treated. The Fyn tyrosine kinase inhibitor may be administered in the form of a pharmaceutical composition with a pharmaceutically acceptable carrier.

As used herein, the term “treating” refers to: (i) preventing a disease, disorder or condition from occurring in an animal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition. For example, with respect to Parkinson's disease, treatment may be measured by quantitatively or qualitatively to determine the presence/absence of the disease, or its progression or regression using, for example, symptoms associated with the disease or clinical indications associated with the pathology.

As used herein, the term “polypeptide” is interpreted to mean a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. “Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well-known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADPribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-link formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1 12 in POSTTRANSLATIONAL COVALENT MODIFICATION OF PROTEINS, B. C. Johnson, Ed., Academic Press, New York (1983); Seifter et al., Meth. Enzymol. 182:626 646 (1990) and Rattan et al., Protein Synthesis: Posttranslational Modifications and Aging, Ann. N.Y. Acad. Sci. 663: 48 62 (1992). Polypeptides may be branched or cyclic, with or without branching. Cyclic, branched and branched circular polypeptides may result from post-translational natural processes and may be made by entirely synthetic methods, as well.

As used herein, the term “polynucleotide” is interpreted to mean a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces T.

Methods for Treating or Preventing Neuroinflammation

The present invention relates generally to compositions and methods for treating and preventing Fyn kinase-modulated disease in a subject in need thereof. The invention is useful, for example, for slowing, protecting from the effects of, or halting the progression of a neuroinflammatory disease. In certain embodiments, the disease or disorder contemplated within the invention is associated with pathological Fyn kinase.-mediating signaling. Non-limiting examples of Fyn kinase-modulated disease that are treatable or preventable with the compositions and methods of the present invention include, but are not limited to, Parkinson's Disease, Alzheimer's Disease (AD), amnestic mild cognitive impairment (MCI), Down syndrome dementia, traumatic brain injury, Lewy body dementia, fronto-temporal dementia, and after stroke aphasia. It should be noted that the present invention is not limited to a particular type of Fyn kinase-modulated disease. The subject may be an animal, preferably a mammal or human.

The present invention also provides a pharmaceutical composition for treating an Fyn kinase-modulated disease in a subject, wherein the composition comprises an inhibitor of Fyn receptor kinase activity and a carrier. For example, in certain embodiments, the composition comprises an isolated nucleic acid, isolated peptide, antibody, small molecule, antagonist, aptamer, or peptidomimetic that reduces the activity of Fyn.

In other embodiments, the present invention provides a composition for treating an Fyn kinase-modulated disease in a subject, wherein the composition comprises an inhibitor of Fyn tyrosine receptor kinase expression and a carrier. For example, in certain embodiments, the composition comprises an isolated nucleic acid (e.g., siRNA, ribozyme, antisense RNA, etc.) that reduces the expression level of Fyn receptor kinase in a cell.

In certain embodiments, the Fyn receptor kinase inhibitor is prepared as a prodrug. A prodrug is an agent converted into the parent drug in vivo. In one embodiment, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound. Prodrugs are known to those skilled in the art, and may be prepared using methodology described in the art. Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, the isotope comprises deuterium. In certain embodiments, isotopically labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N is useful in Positron Emission Topography (PET) studies for examining substrate receptor occupancy. Isotopically labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

In one embodiment the inhibitor is a small molecule inhibitor of Fyn tyrosine selected from the group consisting of saracatinib, bosutinib, dasatinib, ponatinib, PP2, a pharmaceutically acceptable salt thereof, a derivative thereof, and any combinations thereof. In a preferred embodiment the small molecule inhibitor is saracatinib, otherwise known as AZD0530, or N-(5-chlorobenzo[d][1,3]dioxol-4-yl)-7-(2-(4-methyl piperazin-1-yl)ethoxy)-5-(tetrahydro-2H-pyran-4-yloxy)quinazolin-4-amine)-, or pharmaceutically acceptable salts, prodrugs, solvates or derivatives thereof.

Saracatinib (AZD530), has the following chemical formula:

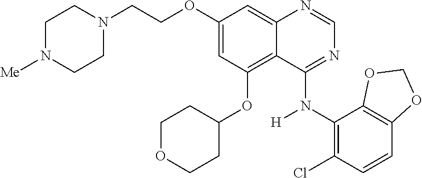 [SMILES: CN1CCN(CCOc2cc(OC3CCOCC3)c3c(Nc4c(Cl)ccc5c4OCO5)ncnc3c2)CC1]

N-(5-chloro-1,3-benzodioxol-4-yl)-7-[2-(4-methylpiperazin-1-yl)ethoxy]-5-(oxan-4-yloxy)quinazolin-1-amine and is commercially available through Astra Zeneca. The compound is currently used to treat advanced solid tumors in pancreatic, ovarian, prostate, osteosarcoma, melanoma, and colon cancer.

Thus the invention provides a pharmaceutical composition comprising an effective amount of saracatinib, or a pharmaceutically acceptable salt, solvate or prodrug thereof, and a carrier.

In some embodiments an effective amount can include an amount sufficient for a trough cerebral spinal fluid (CSF) concentration of saracatinib in the mammal of at least about 0.9 nM. In other embodiments, the trough CSF concentration of saracatinib in the mammal is at least about 2.1 nM. In yet other embodiments, the trough CSF concentration of saracatinib in the mammal is at least about 2.5 nM. In yet other embodiments, the trough CSF concentration of saracatinib in the mammal ranges from about 0.9 nM to about 2.2 nM. In yet other embodiments, the trough CSF concentration of saracatinib in the mammal ranges from about 2.1 nM to about 8.3 nM. In yet other embodiments, the trough CSF concentration of saracatinib in the mammal ranges from about 2.5 nM to about 14.0 nM. In yet other embodiments, the trough CSF concentration of saracatinib in the mammal ranges from about 0.9 nM to about 14.0 nM. In yet other embodiments, the average brain concentration of saracatinib in the mammal is selected from the group consisting of at least about 3 nM, at least about 7 nM, and at least about 8 nM. In yet other embodiments, the average brain concentration of saracatinib in the mammal ranges from about 3 to about 46 nM.

In certain embodiments, the saracatinib is saracatinib free base. In other embodiments, the saracatinib is saracatinib difumarate. In yet other embodiments, the saracatinib is selected from the group consisting of saracatinib free base, saracatinib difumarate, and any combinations thereof.

In certain embodiments, the methods and compositions further comprise administering to the mammal at least one additional or a second agent that treats or prevents Parkinson's or other neurological disease states. In yet other embodiments, the method further comprises administering to the mammal at least one additional agent that improves or prevents further neurodegeneration in the mammal. Pharmaceutical compositions of the invention can include an additional agent that is co-formulated with the Fyn tyrosine receptor inhibitor.

Examples of additional agents that treat or prevent Parkinson's disease or neurodegeneration and may be co-administered according to the invention include but are not limited to:

Carbidopa—currently the most effective Parkinson's disease medication. It is a natural chemical that is converted to dopamine;

Levodopa which is combined with carbidopa (Rytary, Sinemet), which protects levodopa from premature conversion to dopamine outside of the brain, which prevents or lessens side effects such as nausea;

Carbidopa-levodopa infusion, Duopa: carbidopa and levodopa which is administered through a feeding tube that delivers the medication in a gel form directly to the small intestine;

Dopamine agonists: Agents which mimic dopamine effects in the brain. Examples of dopamine antagonists include pramipexole (Mirapex), ropinirole (Requip) and rotigotine (given as a patch, Neupro). A short-acting injectable dopamine agonist, apomorphine (Apokyn), is used for quick relief;

MAO-B inhibitors: These medications include selegiline (Eldepryl, Zelapar) and rasagiline (Azilect). They help prevent the breakdown of brain dopamine by inhibiting the brain enzyme monoamine oxidase B (MAO-B). This enzyme metabolizes brain dopamine.

Catechol-O-methyltransferase (COMT) inhibitors. Entacapone (Comtan) is the primary medication from this class. This medication mildly prolongs the effect of levodopa therapy by blocking an enzyme that breaks down dopamine. Tolcapone (Tasmar) is another COMT inhibitor that is rarely prescribed due to a risk of liver damage anticholinergic medications such as benztropine (Cogentin) or trihexyphenidyl.

Amantadine. provides short-term relief of symptoms of mild, early-stage Parkinson's disease. It may also be given with carbidopa-levodopa therapy during the later stages of Parkinson's disease to control involuntary movements (dyskinesias) induced by carbidopa-levodopa.

In another aspect of the invention the Fyn kinase inhibitor may be combined with surgical procedures. One example is deep brain stimulation. In deep brain stimulation (DBS), surgeons implant electrodes into a specific part of the brain. The electrodes are connected to a generator implanted in the chest near the collarbone that sends electrical pulses to the brain and can reduce Parkinson's disease symptoms. DBS can stabilize medication fluctuations, reduce or halt involuntary movements (dyskinesias), reduce tremor, reduce rigidity, and improve slowing of movement.

In another aspect of the invention the Fyn tyrosine kinase inhibitor is a polynucleotide, polynucleotide that includes but is not limited to an antisense polynucleotide, ribozyme, RNA interference (RNAi) molecule, small hairpin RNA (shRNA), triple helix polynucleotide and the like, where the nucleotide sequence of such polynucleotides are the nucleotide sequences of DNA and/or RNA. Antisense technology may be used to achieve Fyn tyrosine kinase-specific interference, using for example, stoichiometric amounts of single-stranded nucleic acid complementary to the messenger RNA of Fyn tyrosine kinase which are introduced into the cell.

In one embodiment, an RNA interference (RNAi) molecule is used as a Fyn tyrosine kinase inhibitor, decreasing Fyn tyrosine kinase gene expression in a cell. In another aspect, the Fyn tyrosine kinase inhibitor is a siRNA molecule for targeting Fyn tyrosine kinase in a mammal, including without limitation, Fyn tyrosine kinase siRNA for a mouse, rat, monkey, or human.

In one embodiment, the compositions and methods of the present invention includes a Fyn tyrosine kinase inhibitor of at least one Fyn tyrosine kinase siRNA. In one aspect, the Fyn tyrosine kinase inhibitor includes a combination of differing Fyn tyrosine kinase siRNA molecules. Materials and methods to produce Fyn tyrosine kinase siRNA molecules are known in the art. The Fyn tyrosine kinase siRNA molecules may be produced by a number of methods, including the use of commercially available kits, for example, The SILENCER™ siRNA Construction Kit (Ambion, Austin, Tex.), a mammalian siRNA Fyn tyrosine kinase expression plasmid (Upstate Cell Signaling Solutions, Charlottesville, Va.), or obtained from MoleculA (Columbia, Md.).

The siRNA can be administered directly, for example, intracellularly, into a cell to mediate RNA interference (Elbashir et al., 2001, Nature 411:494 498) or administered extracellularly into a cavity, interstitial space, into the circulation of an organism, introduced orally, or may be administered by contacting the cell with a solution containing the RNA. Physical methods of introducing polynucleotides, for example, injection directly into the cell or extracellular injection into the organism, may also be used. Other methods known in the art for introducing polynucleotides to cells may be used, such as viral vectors, viruses, lipid-mediated carrier transport, chemical-mediated transport, such as calcium phosphate, electroporation, the like. siRNA can be made using, for example, chemical synthesis or in vitro or in vivo transcription. A number of expression vectors have also been developed to continually express siRNAs in transiently and stably transfected mammalian cells (Brummelkamp et al., 2002 Science 296:550 553; Sui et al., 2002, PNAS 99(6):5515 5520; Paul et al., 2002, Nature Biotechnol. 20:505 508

In one embodiment, the Fyn tyrosine kinase inhibitor is a small hairpin RNA (shRNA), which is processed in vivo into siRNA-like molecules capable of carrying out gene-specific silencing. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. The RNA molecule may be at least 10, 12, 15, 20, 21, 22, 23, 24, 25, 30, nucleotides in length.

RNA containing a polynucleotide sequence identical to a portion of Fyn tyrosine kinase gene is preferred for inhibition. RNA sequences with insertions, deletions, and single point mutations relative to the target sequence of Fyn tyrosine kinase may also be effective for inhibition. Thus, one hundred percent sequence identity between the RNA and the target gene is not required to practice the present invention. Greater than 80% or 90% sequence identity or 100% sequence identity, between the inhibitory RNA and the portion of the Fyn tyrosine kinase is preferred. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Fyn tyrosine kinase inhibitors can be delivered intracellularly using a lipid-mediated protein delivery system. Kaul S, Kanthasamy A, Kitazawa M, Anantharam V, Kanthasamy A G, Caspase-3 dependent proteolytic activation of Fyn tyrosine kinasemediates and regulates 1-methyl-4-phenylpyridinium (MPP⁺)-induced apoptotic cell death in dopaminergic cells: relevance to oxidative stress in dopaminergic degeneration. Eur J Neurosci. 2003 September; 18(6):1387-401. The incomplete cleavage of PKCδ or PKCδ-mediated apoptotic inhibition can be measured using standard techniques known to one skilled in the art. Yoshimura S, Banno Y, Nakashima S, Takenaka K, Sakai H, Nishimura Y, Sakai N, Shimizu S, Eguchi Y, Tsujimoto Y, Nozawa Y. Ceramide formation leads to caspase-3 activation during hypoxic PC12 cell death. Inhibitory effects of Bcl-2 on ceramide formation and caspase-3 activation. J Biol Chem. 1998 Mar. 20; 273(12):6921-7 (describing a method to determine caspase activity). Kaul S, Kanthasamy A, Kitazawa M, Anantharam V, Kanthasamy A G, Caspase-3 dependent proteolytic activation of Fyn tyrosine kinasemediates and regulates 1-methyl-4-phenylpyridinium (MPP⁺)-induced apoptotic cell death in dopaminergic cells: relevance to oxidative stress in dopaminergic degeneration. Eur J Neurosci. 2003 September; 18(6):1387-401) (describing the use of PKCδ-specific antibodies). Reyland M E, Anderson S M, Matassa A A, Barzen K A, Quissell D O. Fyn tyrosine kinase is essential for etoposide-induced apoptosis in salivary gland acinar cells. J Biol Chem. 1999 Jul. 2; 274(27):19115-23), Anantharam V, Kitazawa M, Wagner J, Kaul S, Kanthasamy A G. Caspase-3-dependent proteolytic cleavage of protein kinase C delta is essential for oxidative stress-mediated dopaminergic cell death after exposure to methylcyclopentadienyl manganese tricarbonyl. J Neurosci. 2002 Mar. 1; 22(5):1738-51, (describing assaying for Fyn tyrosine kinase enzymatic activity using an immunoprecipitation assay).

In one embodiment, the compositions and methods of the present invention include disruption of the Fyn-PKCδ pathway, particularly a PKCδ serine- and threonine kinase inhibitor or an RNAi molecule used as a PKCδ serine- and threonine kinase inhibitor. A suitable small molecule inhibitor of PKCδ serine- and threonine kinase can be selected from the group consisting of Balanol (n-tosyl derivative), δV1-1 (KAI-9803 or delcasertib), Rottlerin (5,7-dihydroxy-2,2-dimethyl-6-(2,4,6-trihydroxy-3-methyl-5-acetylbenzyl)-8-cinnamoyl-1,2-chrmene), a pharmaceutically acceptable salt thereof, a derivative thereof, and any combinations thereof. Suitable methods for RNAi molecule design and delivery, targeted for inhibition of PKCδ serine- and threonine kinase, are synonymous with those describe above.

In another embodiment of the invention, other agents such as peptides, small molecules and the like may be screened to identify those that alter Fyn tyrosine kinase activity, particularly the ability of Fyn tyrosine kinase upregulation in response to inflammatory signals such as TNFα or LPB to identify additional Parkinson's disease treating agents. The Fyn tyrosine kinase inhibitor may directly alter the interaction of proteins in the Fyn tyrosine kinase pathway, for example, by inhibiting the activity of a kinase or phosphorylase in the pathway or by interfering with a step of the pathway.

The inhibition or reduction of Fyn tyrosine kinase activity can be determined using a variety of methods and assays routine to one skilled in the art, for example, determining the phosphorylation and/or activation of a Fyn tyrosine kinase kinase's target. Generally, a purified or partially purified Fyn tyrosine kinase is incubated with a peptide comprising the target sequence of Fyn tyrosine kinase under conditions suitable for the kinase to phosphorylate its target sequence of amino acids (i.e., protein, polypeptide). The particular requirements of the kinase may be determined empirically by one of skill in the art, or the conditions that have been published for a particular kinase may be used. The extent of phosphorylation of the target peptide is determined in the presence and absence of the test compound and may be determined in the presence of varying concentrations of the test compound. The phosphorylation rate may be determined by any means known in the art including electrophoretic assays, chromatographic assays, phosphocellulose assays and the like.

In an electrophoretic assay, a radiolabeled phosphate donor such as ATP or GTP is incubated with the peptide substrate in the presence of a kinase. The phosphorylated substrate versus the phosphate donor (e.g., ATP, GTP) is separated via thin-layer electrophoresis (Hunter J. Biol. Chem. 257:4843, 1982; incorporated herein by reference). Any matrix may be used in the electrophoresis step including polyacrylamide, cellulose, etc. The extent of phosphorylation may then be determined by autoradiography or scintillation counting.

The labeled phosphate donor may be separated from the phosphorylated amino acid sequence by standard chromatography techniques. Any matrix may be used to effect the separation including ion exchange resins, PEI cellulose, silica gel, etc. Standard column chromatography methods may be used, or HPLC methods may be used for faster cleaner separations. The radio-labeled peptides are detected by scintillation counting to determine the phosphorylation rate.

Another method which is historically the most popular is the phosphocellulose paper assay, first described by Witt et al. (Witt et al. Anal. Biochem. 66:253, 1975; incorporated herein by reference). Immunological methods may also be used to detect the phosphorylation of a peptide or protein substrate. For example, anti-phosphotyrosine or anti-phosphoserine antibodies may be used in the detection or precipitation of phosphorylated amino acid sequences. For example, multiple Fyn tyrosine kinase antibodies that detect the phosphorylated and unphosphorylated forms of Fyn tyrosine kinase are commercially available. (Cell signaling, Beverly, Mass. and Santa Cruz, Santa Cruz, Calif.).

In comparing the rates of phosphorylation in the presence and absence of the test compound, the compound should lead to at least a 10% decrease in the rate of phosphorylation, more preferably at least 25%, and most preferably at least 40%. These decreases are preferably obtained at micromolar concentrations of the compound and more preferably nanomolar concentrations (e.g., less than 100 nM).

In another aspect, the invention includes determining whether a potential Fyn tyrosine kinase inhibitor inhibits Fyn tyrosine kinase activity. The ability of a Fyn tyrosine kinase inhibitor to decrease Fyn tyrosine kinase activity, for example, neurodegeneration can be assessed using standard techniques known to one skilled in the art, including commercially available assays. These include apoptotic DNA Ladder assays, Cell Death Detection ELISA^{PLUS} (from Roche Applied Sciences), Caspase-3 Activity Assay^{PLUS} (from Roche Applied Sciences), terminal deoxynucleotidyl transferase-mediated dUTP [deoxy-uridine triphosphate] nick end labeling (TUNEL) assays. DNA-binding dyes or stains such as Hoechst 3342 or DAPI or propidium iodide may be used to assess nuclear morphology and DNA damage. Kaul S, Kanthasamy A, Kitazawa M, Anantharam V, Kanthasamy A G, Caspase-3 dependent proteolytic activation of Fyn tyrosine kinasemediates and regulates 1-methyl-4-phenylpyridinium (MPP⁺)-induced apoptotic cell death in dopaminergic cells: relevance to oxidative stress in dopaminergic degeneration. Eur J Neurosci. 2003 September; 18(6): 1387-401.

In one aspect, the method includes administering a Fyn tyrosine kinase inhibitor. A Fyn tyrosine kinase inhibitor may be administered to an individual by various routes including, for example, orally or parenterally, such as intravenously, intramuscularly, subcutaneously, intraorbitally, intracapsularly, intraperitoneally, intrarectally intracisternally or by passive or facilitated absorption through the skin using, for example, a skin patch or transdermal iontophoresis, respectively. Furthermore, a composition comprising a Fyn tyrosine kinase inhibitor can be administered by injection, intubation, orally or topically, the latter of which can be passive, for example, by direct application of an ointment or powder, or active, for example, using a nasal spray or inhalant. A Fyn tyrosine kinase inhibitor also can be administered as a topical spray or an inhalant, in which case one component of the composition is an appropriate propellant.

The skilled artisan can readily perform the in vivo tests to determine, the amount or dose of Fyn tyrosine kinase inhibitor to administer, the formulation of the Fyn tyrosine kinase inhibitor, the route of administration of the Fyn tyrosine kinase inhibitor, and the time at which neurodegeneration or neuroprotection and dopamine synthesis or levels should be assessed.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a concentration range that includes the IC₅₀ (i.e., the concentration of the test compound that achieves a half-maximal inhibition of symptoms) as determined in cell culture. Such information can be used to more accurately determine useful doses in humans. Levels in plasma and cerebral spinal fluid may be measured, for example, by high performance liquid chromatography.

Neurodegeneration or neuroprotection can be detected using any number of methods. Histological, neurochemical and biochemical markers of dopamine producing cells. These techniques are routine and well-known to one skilled in the art. They include, for example, terminal deoxynucleotidyl transferase-mediated dUTP-X3′ nick end-labeling (TUNEL) assays that detect the free 3′ OH strand breaks resulting from DNA degradation which is associated with apoptosis (J Cell Biol 199: 493, 1992). In addition, kits that measure apoptotic cell death are also commercially available and include, for example, In Situ Cell Death Detection kit; Boehringer Mannheim, Mannheim or ApoTag, Oncor, Gaithersburg, Md.). Preparation of neuronal sections for apoptosis staining using the TUNEL technique is described in (Gorczyca, (1993) Cancer Res 53:1945-51). Apoptosis can also be detected using electrophoresis of the soluble DNA fraction isolated from neuronal cells by quantifying the ladder-like appearance as described in (PNAS 95: 2498, 1998) or using DNA binding dyes Hoechst 33342 and propidium iodide flow cytometry assay described in Dengler et al., (1995) Anticancer Drugs. 6:522-32.

The present invention also provides kits that can be used in the above methods. In one embodiment, a kit comprises at least one Fyn tyrosine kinase inhibitor, and optimally a second treating agent, in one or more containers, useful for the treatment of a disorder, disease, or condition associated with neurodegeneration or Parkinson's disease. Optionally associated with such container(s) can be a notice in the form prescribed by a governmental agency regulating the manufacture, use or sale of pharmaceuticals or biological products, which notice reflects approval by the agency of manufacture, use or sale for human administration.

All publications, patents and patent applications identified herein are incorporated by reference, as though set forth herein in full. The invention being thus described, it will be apparent to those skilled in the art that the same may be varied in many ways without departing from the spirit and scope of the invention. Such variations are included within the scope of the following claims.

The invention is further illustrated by the following specific examples which are not intended in any way to limit the scope of the invention.

### Example 1

Sustained neuroinflammation mediated by resident microglia is recognized as a key pathophysiological contributor to many neurodegenerative diseases, including Parkinson's disease (PD), but the key molecular signaling events regulating persistent microglial activation have yet to be clearly defined. While studying kinase signaling in PD models, Applicants unexpectedly discovered that the major non-receptor tyrosine kinase Fyn is a significant upstream mediator of microglial neuroinflammatory processes in PD. Applicants herein elucidate the role of Fyn in microglial activation and neuroinflammatory mechanisms in cell culture and animal models of PD. The well-characterized inflammogens lipopolysaccharide (LPS) and tumor necrosis factor alpha (TNFα) rapidly activated Fyn kinase in microglia. Immunocytochemical studies revealed that activated Fyn preferentially localized to the microglial plasma membrane periphery and the nucleus. Furthermore, activated Fyn phosphorylated PKCδ at tyrosine residue 311, contributing to an inflammogen-induced increase in its kinase activity. Notably, the Fyn-PKCδ signaling axis further activated the LPS- and TNFα-induced MAP kinase phosphorylation and activation of the NFκB pathway, implying that Fyn is a major upstream regulator of the proinflammatory signaling. Functional studies in microglia isolated from wild-type (Fyn+/+) and Fyn knockout (Fyn−/−) mice revealed that Fyn is required for the pro-inflammatory responses, including cytokine release as well as iNOS activation. Interestingly, a prolonged inflammatory insult induced Fyn transcript and protein expression, indicating that Fyn is upregulated during chronic inflammatory conditions. Importantly, in vivo studies using MPTP, LPS, or 6-OHDA models revealed a greater attenuation of neuroinflammatory responses in Fyn−/− and PKCδ−/− mice when compared to wild-type mice.

The pathophysiology of PD is complex and multifactorial, with mitochondrial dysfunction, oxidative stress, apoptosis and proteasomal dysfunction, being identified among others as potential disease mechanisms underlying nigrostriatal dopaminergic neuronal degeneration (Przedborski, 2005; Jenner and Olanow, 2006; Olanow, 2007; Levy et al., 2009). Recently, a wealth of data from cell culture, animal models and post-mortem analyses of human PD brains have established chronic, sustained microglia-mediated neuroinflammation as being a major event in the delayed and progressive loss of dopaminergic neurons within the SN (Imamura et al., 2003; Block et al., 2007; Glass et al., 2010; Tansey and Goldberg, 2010). As the macrophagic cells of the central nervous system (CNS), microglia compose a major component of the brain's innate immune system. Under ‘normal’ physiological conditions, they produce anti-inflammatory and neurotrophic factors to promote neuronal survival and plasticity (Carson, 2002). However, when they encounter a potential pathogen, a dead or dying neuron or neurotoxic stress, they switch to an ‘activated’ phenotype, producing pro-inflammatory cytokines and chemokines, reactive nitrogen species, and reactive oxygen species. Activated microglia may also directly contribute to cell death by phagocytizing dopaminergic neurons (Barcia et al., 2012; Virgone-Carlotta et al., 2013). Thus, the pathophysiology of PD is accompanied by a sustained pro-inflammatory microglial response that contributes to neuron death, thereby exacerbating disease progression.

Fyn, a member of the Src family of kinases, is a non-receptor tyrosine kinase expressed in the brain. The kinase has been shown to play a role in amyloid-mediated apoptosis in cortical neurons (Lambert et al., 1998), astrocyte migration (Dey et al., 2008) and oligodendrocyte differentiation (Sperber et al., 2001). In the peripheral immune system, Fyn plays a role in mast cell and B/T cell activation (Palacios and Weiss, 2004; Gomez et al., 2005a). Fyn was shown to mediate pro-inflammatory mediator production in mast cells, macrophages, basophils as well as in natural killer cells (Raj asekaran et al., 2013). Fyn was shown to be activated following fibrillar β-amyloid peptide engagement of its receptor CD36, contributing to activation and migration of primary murine peritoneal macrophages (Moore et al., 2002; Stuart et al., 2007), and in BV2 microglial cells stimulated with the neurotoxic fragment of prion protein (Kouadir et al., 2012). Recently, we have identified a pro-apoptotic Fyn/PKCδ-mediated signaling pathway that contributes to oxidative stress-induced cell death in dopaminergic neurons (Kaul et al., 2005; Saminathan et al., 2011). However, the role of Fyn in microglial activation and neuroinflammation has never been studied in PD. Therefore, Applicants sought to characterize the role of the Fyn-PKCδ signaling pathway in microglial activation and neuroinflammation in cell culture and animal models of PD. The results from these comprehensive studies reveal that Fyn kinase plays a key role in microglial activation and sustained neuroinflammation in the nigral dopaminergic system.

### Results

### Fyn and PKCδ are Differentially Expressed in Primary Astrocytes and Microglia

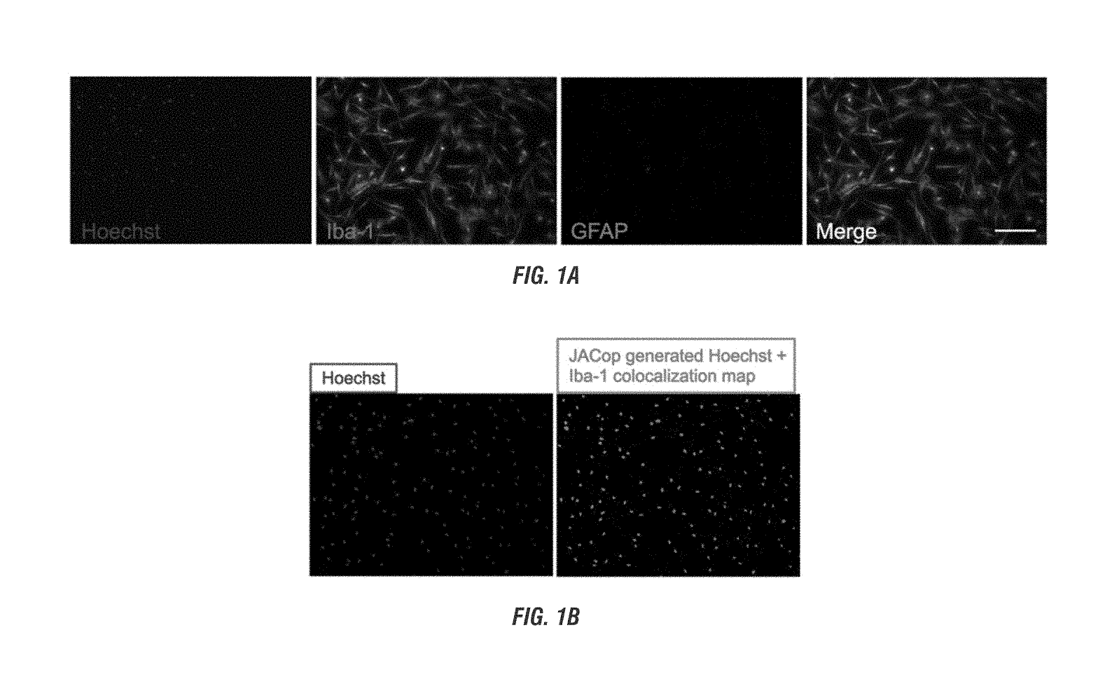

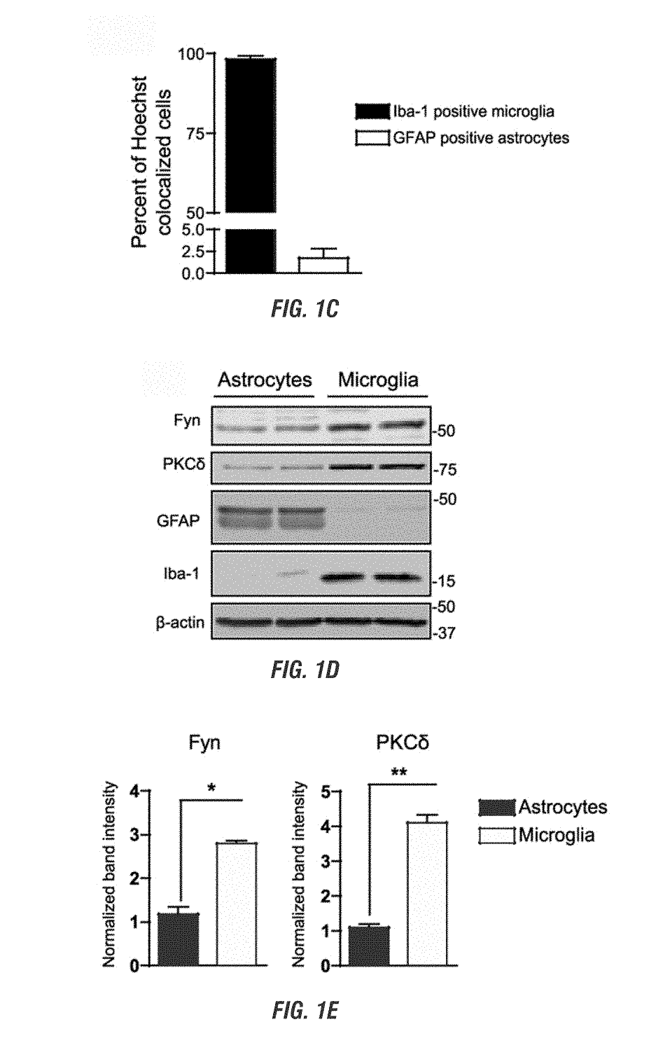

Primary mouse microglia were prepared as described in our recent publication using a magnetic separation method, which enables us to obtain a high-yield pure fraction of microglia from mixed glial cultures (Gordon et al., 2011). Iba-1 and GFAP immunocytochemistry confirmed that the microglial fraction obtained after magnetic separation was devoid of astrocytes (FIG. 1A). Quantification of Hoechst co-localized Iba-1-positive microglia and GFAP-positive astrocytes using the ImageJ plugin JACoP revealed a microglial population that was 97% pure post-separation (FIGS. 1B-C). Immunoblotting analysis revealed that microglia-enriched fractions expressed significantly more Fyn (60 kDa) and PKCδ (76 kDa) than did astrocyte-enriched (microglia-depleted) fractions (FIGS. 1D-E). The differential expression of both Fyn and PKCδ in microglia compared to astrocytes prompted us to study the roles these proteins may play in microglial pro-inflammatory signaling.

### Fyn Kinase is Rapidly Activated in Microglial Cells and in the Ventral Midbrain Following Inflammogen Stimulation

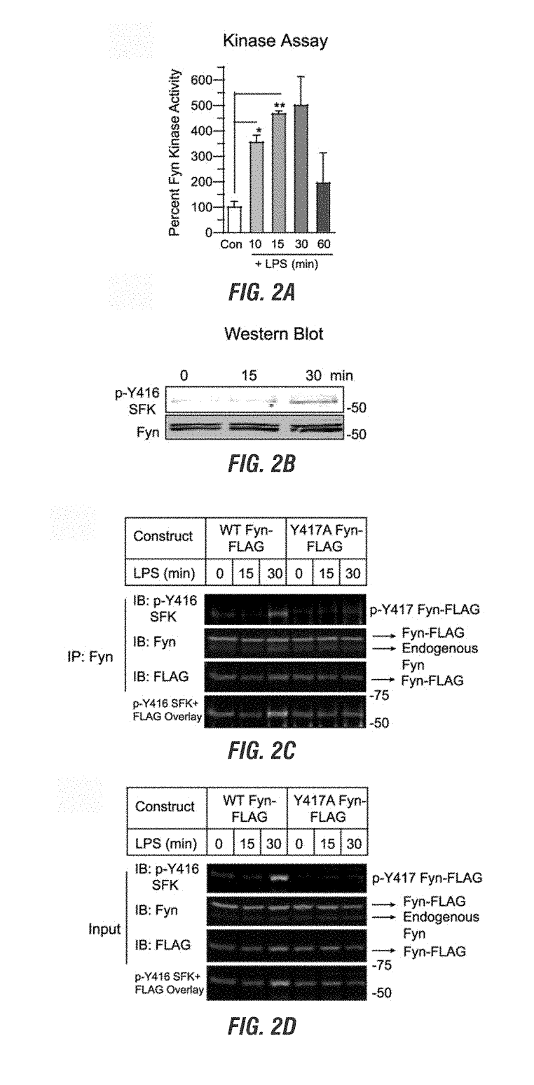

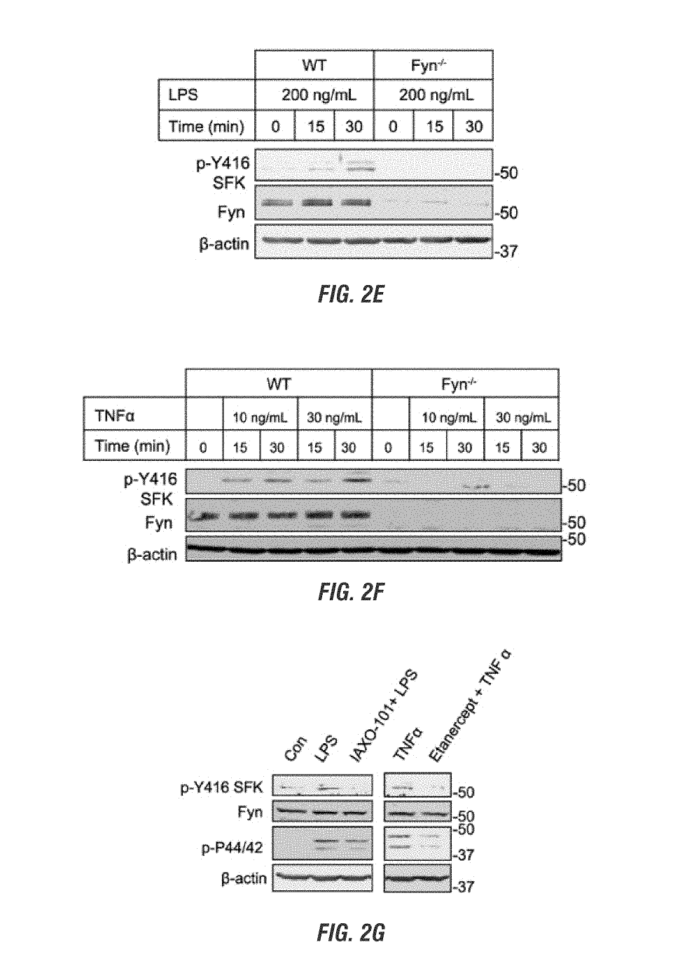

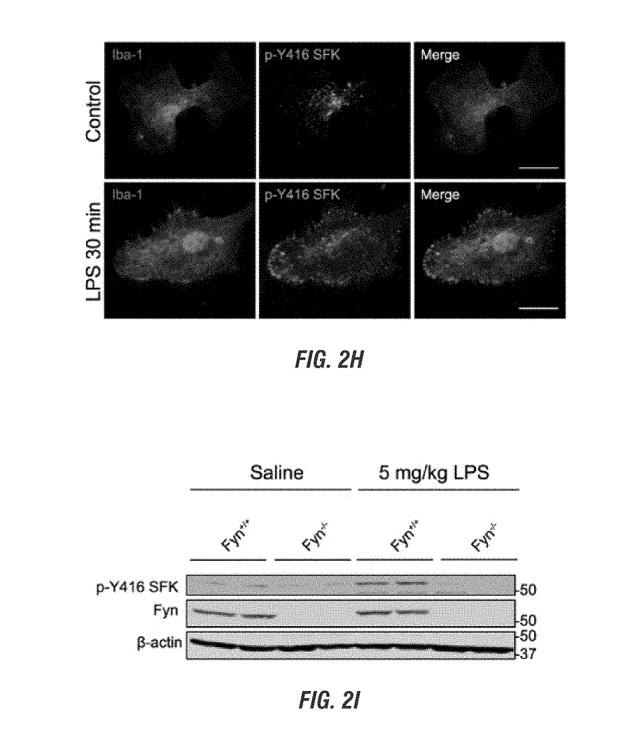

Our initial experiment to determine whether the non-receptor tyrosine kinase Fyn plays a role in regulating neuroinflammatory responses in PD was carried out in BV2 microglial cells, which are widely used in vitro models of neuroinflammation (Henn et al., 2009; Gao et al., 2011; Kim et al., 2013b). We treated BV2 cells with 1 μg/mL LPS for 10-60 min and measured Fyn activity using an in vitro kinase assay (Saminathan et al., 2011). A kinase reaction mixture containing ³²P-ATP and a Fyn-specific peptide substrate were added to whole cell lysates. LPS stimulation of BV2 microglia rapidly induced Fyn activity as early as 10 min post-LPS stimulation (FIG. 2A), and maximal activity was attained 30 min post-LPS stimulation. In addition to the Fyn kinase activity assay, we also determined the phosphorylation status of the Y416 residue in its activation loop domain, by utilizing the phospho Y416 Src family kinase (p-Y416 SFK) antibody, which recognizes activated Src family kinases. This antibody has been used extensively to demonstrate Fyn kinase activation (Larson et al.; Um et al.; Wake et al., 2011; Kouadir et al., 2012). Our immunoblotting analysis of LPS-treated BV2 lysates using the p-Y416 SFK antibody revealed LPS-induced SFK activation (FIG. 2B). To further confirm inflammogen-induced Fyn activation in BV2 microglia, we transiently transfected BV2 cells with FLAG-tagged WT-Fyn and Y417A-Fyn (activation loop mutant) constructs. We then performed immunoprecipitation studies in LPS-treated transfected BV2 cells. We pulled down Fyn from FLAG-tagged WT-Fyn and Y417A-Fyn transfected, LPS-treated BV2 cells and immunoblotted for p-Y416 SFK levels. A strong p-Y416 SFK signal was detected in the LPS-treated WT-Fyn-FLAG-transfected cells, but not in the LPS-treated Y417A-Fyn-transfected cells (FIGS. 2C-D).

Next, we extended our studies to primary microglia derived from both wild-type and Fyn-deficient (Fyn^{-/-}) mice. These were treated with 200 ng/mL LPS for 0-30 min. In line with the analyses of BV2 cells, stimulation of the primary microglia from Fyn^{+/+} mice rapidly increased the levels of p-Y416 SFK (FIG. 2E). Interestingly, p-Y416 SFK was not detected in LPS-treated Fyn^{-/-} microglia, suggesting that LPS preferentially induces Fyn phosphorylation in microglia over other Src family kinases. Treatment of wild-type and Fyn-deficient microglia with TNFα also yielded similar results. Both 10 ng/mL and 30 ng/mL TNFα treatments induced similar levels of p-Y416 SFK in wild-type, but not in Fyn-deficient microglia (FIG. 2F). Pretreatment of wild-type microglia with either the TLR (Toll-like Receptor) antagonist IAXO-101 or the TNFα signaling antagonist Etanercept significantly attenuated both LPS- and TNFα-mediated Fyn activation, respectively (FIG. 2G). We also examined subcellular localization of activated Fyn following LPS stimulation. The Iba-1/p-Y416 SFK double-immunocytochemical analysis showed that LPS treatment dramatically increased p-Y416 Fyn levels in WT primary microglia (FIG. 2H). Active Fyn seems to be preferentially expressed at the periphery of the microglia, possibly allowing it to become activated quickly in response to a pro-inflammatory stimulus. Additionally, activated Fyn was also found in the nucleus of LPS-treated primary microglia. Next, we wanted to confirm that LPS-treatment would activate Fyn in the substantia nigra of mice. Knowing that a single intraperitoneal LPS injection elicits microglial cell activation in the substantia nigra (Qin et al., 2007), we challenged Fyn^{+/+} and Fyn^{-/-} mice with 5 mg/kg LPS or sterile PBS vehicle intraperitoneally for 3 h. Immunoblot analysis of ventral midbrain lysates revealed that LPS significantly increased p-Y416 SFK levels in wild-type compared to saline control, whereas LPS failed to increase p-Y416 SFK levels in the Fyn^{-/-} ventral midbrain lysates. (FIG. 2I). These studies indicate that stimulating microglia with inflammatory stimuli rapidly activates Fyn kinase in both cell culture and animal models of neuroinflammation.

### Fyn Contributes to LPS- and TNFα-Induced Tyrosine Phosphorylation and Activation of PKCδ in Primary Microglia

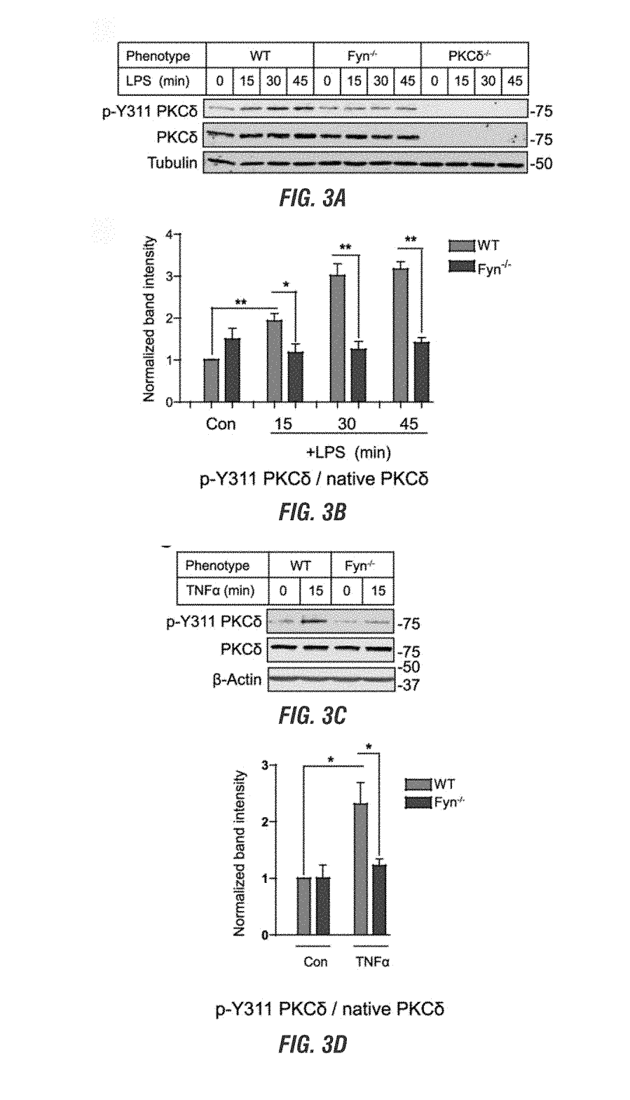

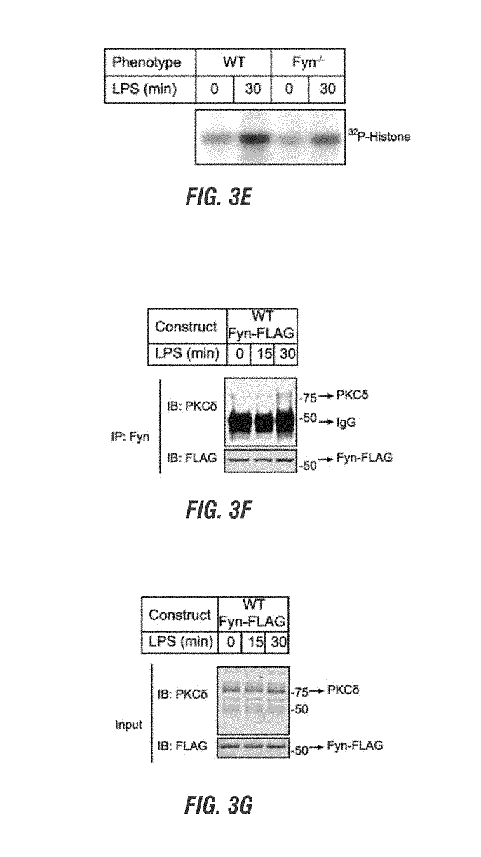

It has been shown that Src family kinases, including Fyn, phosphorylate PKCδ at residue Y311 in platelets and in immortalized dopaminergic neuronal cells (Steinberg, 2004; Saminathan et al., 2011). Therefore, we investigated if Fyn-PKCδ signaling regulates microglial pro-inflammatory responses using primary microglia cultures from wild-type, Fyn^{-/-} and PKCδ^{-/-} mice. Stimulation with LPS induced a rapid and time-dependent increase in p-Y311 PKCδ in wild-type microglia. In contrast, LPS failed to increase Y311 phosphorylation of PKCδ in the Fyn^{-/-} microglia (FIGS. 3A-B). Similarly, TNFα stimulation of microglia also increased PKCδ Y311 phosphorylation in wild-type, but not in Fyn-deficient primary microglia (FIGS. 3C-D). As expected, immunoblot analysis did not detect any LPS-induced phosphorylation of Y311 PKCδ in PKCδ^{-/-} microglia. To confirm further that Fyn mediates the activation of PKCδ in activated microglia, we measured PKCδ kinase activity in wild-type and Fyn^{-/-} microglia. An in vitro PKCδ kinase assay showed that LPS rapidly increased PKCδ kinase activity in wild-type microglia; however, LPS-induced PKCδ kinase activity was significantly less in Fyn^{-/-} microglia (FIG. 3E). To further confirm the Fyn-PKCδ interaction, we performed co-immunoprecipitation studies in BV2 cells transfected with the WT-Fyn-FLAG construct. As shown in FIGS. 3F-G from the co-immunoprecipitation results, co-IP analysis of WT-Fyn-FLAG transfected lysates revealed that Fyn and PKCδ interact during LPS stimulation. Taken together with the PKCδ kinase activity results, these data reveal that Fyn kinase mediates LPS- and TNFα-induced activation of PKCδ in primary microglia.

### The Fyn-PKCδ Signaling Axis Mediates MAP Kinase Activation in Microglial Cells

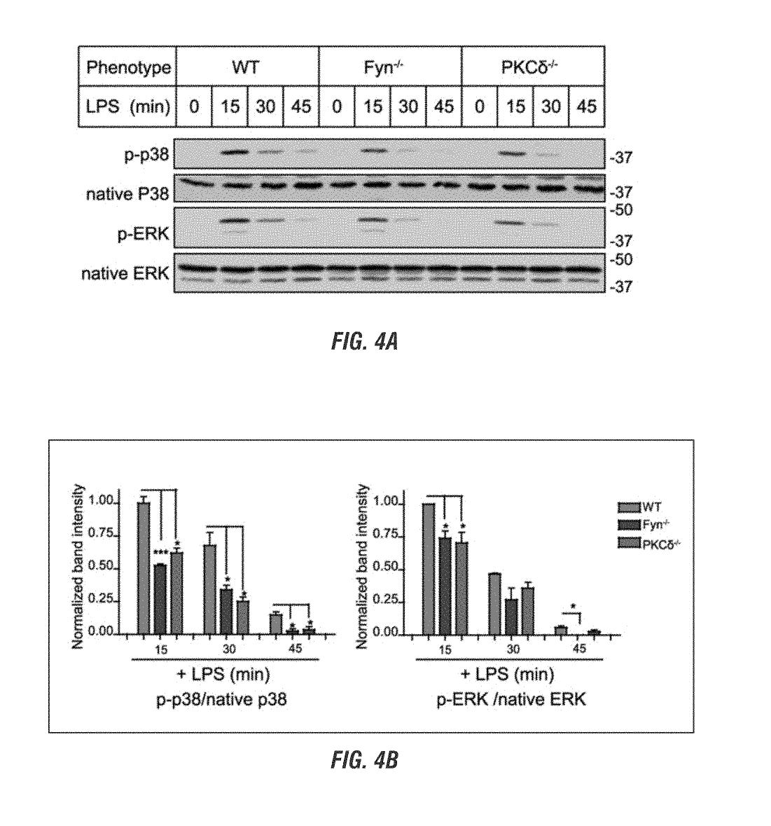

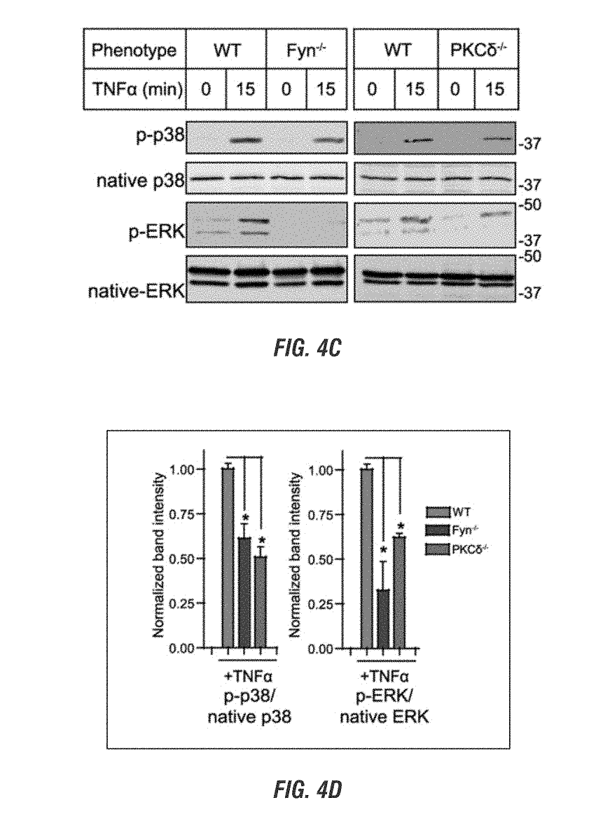

We next examined whether the Fyn-PKCδ signaling axis plays a role in mediating activation of the MAP kinase pathway, a key hallmark of neuroinflammatory signaling in microglia. MAP kinases are important regulators of pro-inflammatory cytokine synthesis in microglial cells (Koistinaho and Koistinaho, 2002; Tansey and Goldberg, 2010). For this purpose, we treated wild-type, Fyn^{-/-}, and PKCδ^{-/-} microglia with LPS for 15, 30 and 45 min each and determined MAPK activation. The LPS treatment significantly increased the phosphorylation of p38 and p44/42 (p-ERK) kinases in wild-type microglia (FIGS. 4A-B), with LPS-induced phosphorylation peaking at 15 min and decreasing thereafter. In contrast, LPS-induced phosphorylation of p38 and p44/42 (p-ERK) was significantly reduced in Fyn^{-/-} and PKCδ^{-/-} primary microglia. Similar results were obtained with TNFα treatment of wild-type, Fyn^{-/-} and PKCδ^{-/-} microglia (FIGS. 4C-D). These results suggest that Fyn-PKCδ signaling is an important upstream regulator of MAP kinases in microglia during both LPS and TNFα stimulation.

### Fyn Contributes to Inflammogen-Mediated NFκB Pathway Activation in Microglial Cells

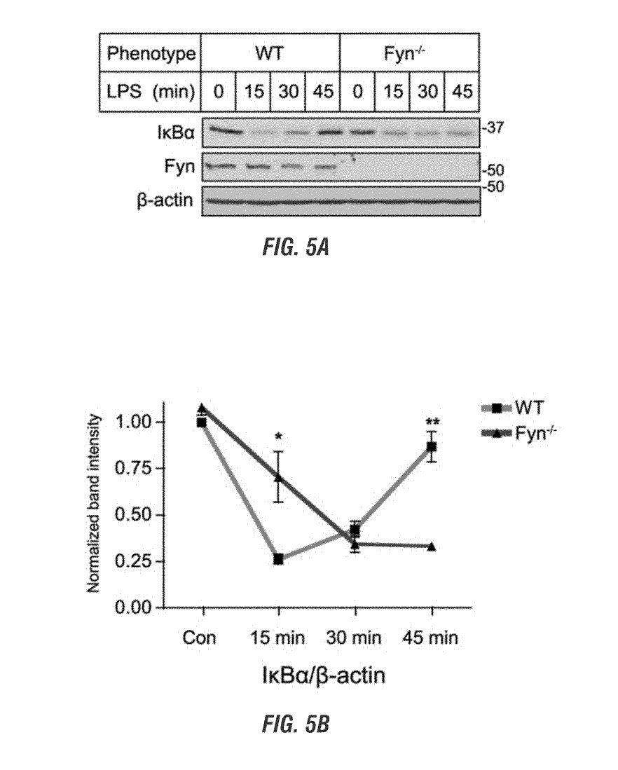

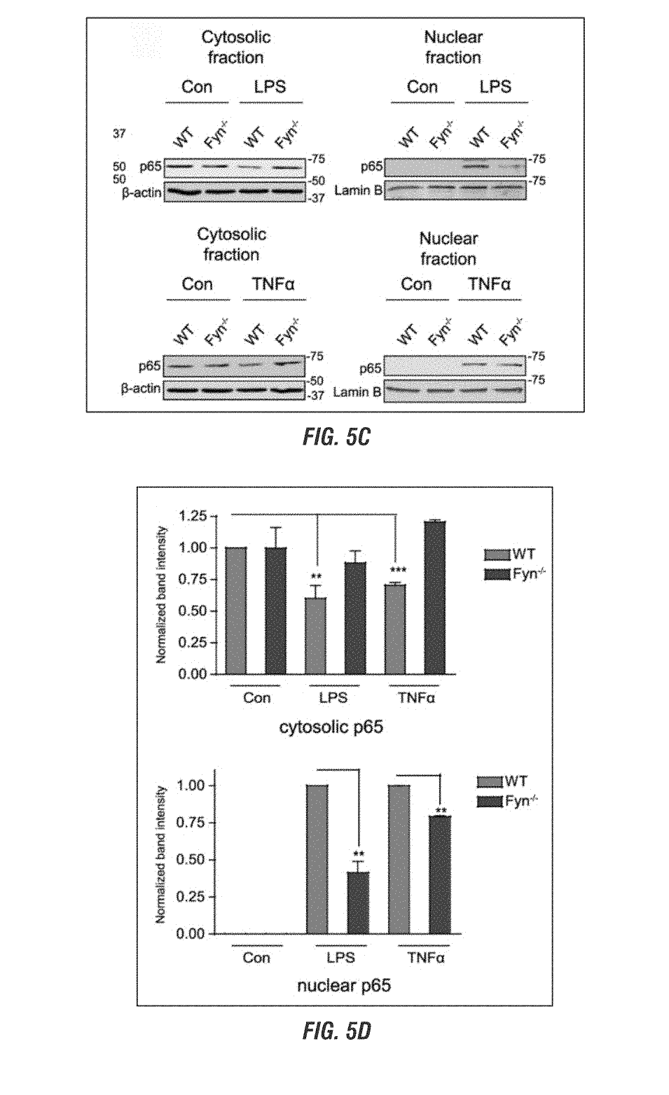

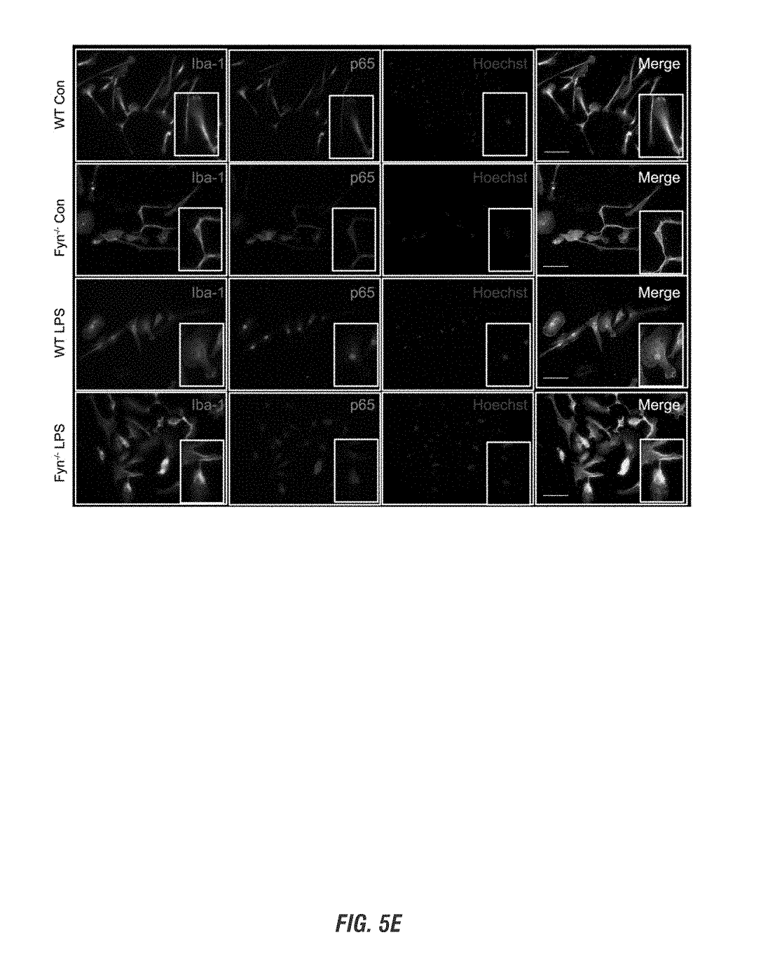

Pro-inflammatory signaling mediated by both LPS and TNFα converges at the NFκB pathway. Activation of NFκB signaling during the pro-inflammatory process is characterized by the phosphorylation and subsequent degradation of the inhibitory protein IκBα, after which the NFκB p65-p50 heterodimer enters the nucleus, leading to the transcription of various pro-inflammatory genes (Hayden and Ghosh, 2004). To elucidate whether the Fyn mediates the nuclear translocation and activation of NFκB signaling in activated microglia, primary microglia obtained from wild-type and Fyn^{-/-} microglia were treated with LPS for 15-45 min. Whole cell lysates were prepared and probed for IκBα. LPS treatment induced a greater degradation of IκBα in wild-type microglia than in Fyn^{-/-} microglia at the 15 minute time point, followed by the resynthesis of IκBα 30 and 45 minutes post stimulation in the WT cells. Resynthesis of IκBα in Fyn^{-/-} microglia was almost completely abrogated, indicating diminished NFκB activation (FIGS. 5A-B). Next, we investigated the role of Fyn in the nuclear translocation of the p65 component of the NFκB complex in response to LPS and TNFα treatments. Nuclear and cytoplasmic fractions were prepared from WT and Fyn^{-/-} microglia treated with LPS or TNFα for 15 min before being assessed for p65 content. Immunoblotting revealed lesser nuclear translocation of p65 in LPS- and TNFα-treated Fyn^{-/-} microglia than in wild-type microglia (FIGS. 5C-D). These results were further supported by Iba-1/p65 double-immunocytochemistry showing strong LPS-induced nuclear translocation of p65 in wild-type, but not in the Fyn^{-/-} microglia (FIG. 5E). Together, these results clearly suggest that Fyn kinase regulates NFκB activation in microglial cells.

### LPS- or TNFα-Induced Pro-Inflammatory Cytokine Production is Suppressed in Fyn/PKCδ Deficient Microglia

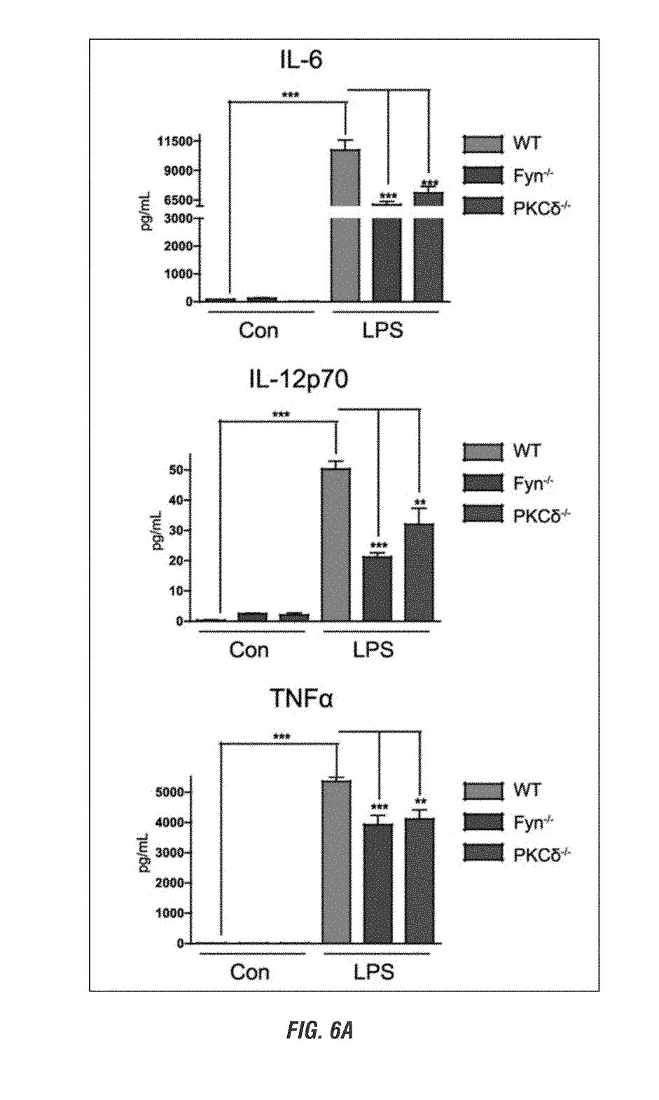

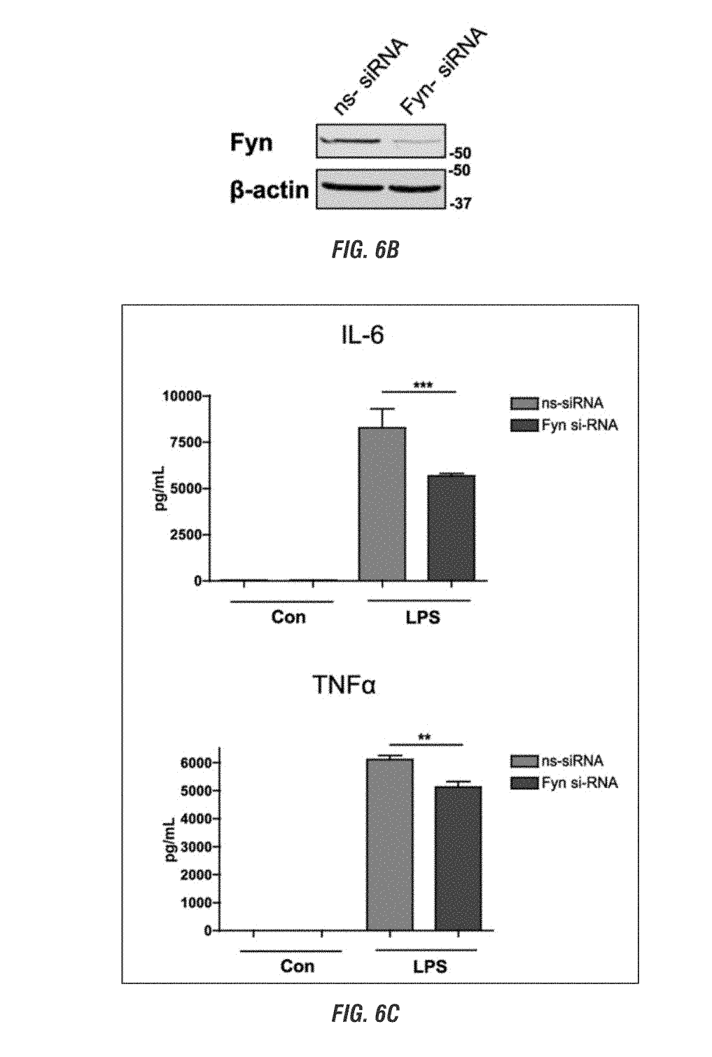

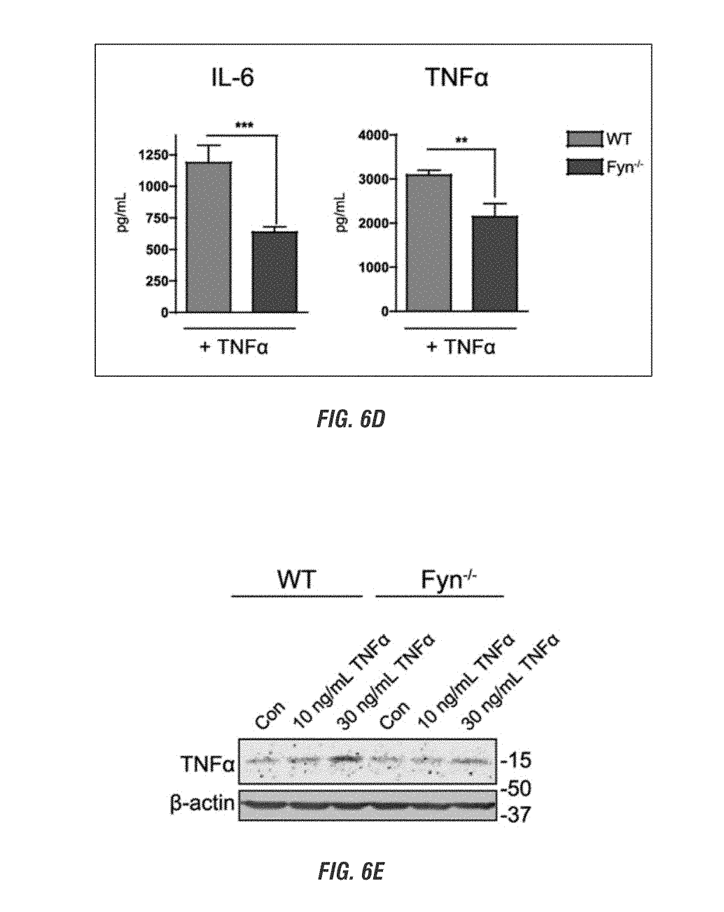

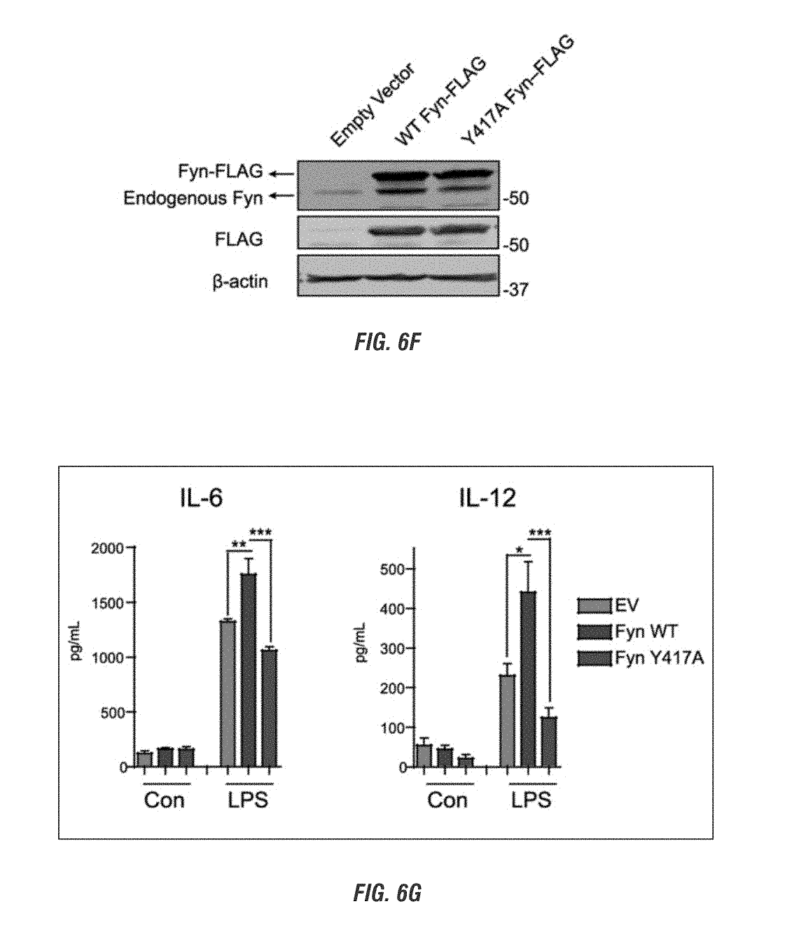

Next, we determined whether Fyn-PKCδ signaling axis regulates microglia-mediated pro-inflammatory mediator production. After treating wild-type, PKCδ^{-/-} and Fyn^{-/-} microglial cultures with LPS or TNFα, we utilized multiplexed immunoassays to quantify inflammogen-induced cytokine secretion. We observed significant production of the cytokines IL-6, IL-12p70, and TNFα from wild-type microglia treated with LPS (FIG. 6A). However, the production of these cytokines was significantly dampened in Fyn- and PKCδ-deficient microglia, providing evidence for the hypothesis that attenuated pro-inflammatory signaling in Fyn^{-/-} and PKCδ^{-/-} microglia suppresses pro-inflammatory mediator production. When we knocked down Fyn expression in wild-type primary microglia by Fyn-specific siRNA (FIG. 6B), diminished amounts of the pro-inflammatory cytokines IL-6 and TNFα were produced in response to LPS treatment (FIG. 6C). Next, treatment of wild-type and Fyn^{-/-} microglia with TNFα yielded similar results, with the Fyn^{-/-} microglia showing reduced IL-6 and TNFα production (FIG. 6D). Western blot analysis also demonstrated that Fyn-deficient microglia produced less TNFα relative to wild-type microglia (FIG. 6E). To further confirm the role of Fyn in pro-inflammatory cytokine production, we expressed Fyn wild-type (WT-Fyn-FLAG) or activation loop mutant (kinase deficient Fyn kinase, Y417A Fyn-FLAG) in BV2 microglial cells (FIG. 6F). Following the transfection, BV2 cells transfected WT-Fyn-FLAG, Y417A Fyn-FLAG or empty vector constructs were treated with 1 μg/mL LPS for 24 h. Luminex immunoassay of cell supernatants revealed that overexpressing wild-type Fyn augmented pro-inflammatory cytokine release, whereas overexpressing the inactive Y417A Fyn mutant suppressed the production of IL-6 and IL-12 (FIGS. 6F-G).

Fyn/PKCδ Regulates the Induction of Neuroinflammatory Markers iNOS and gp91^{phox} in Microglia During LPS Stimulation

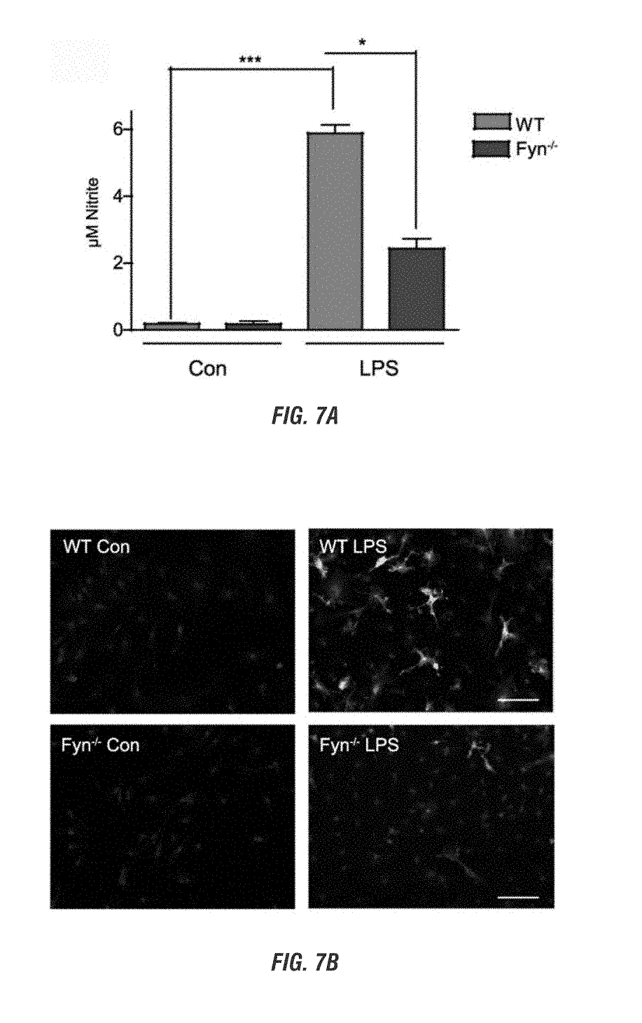

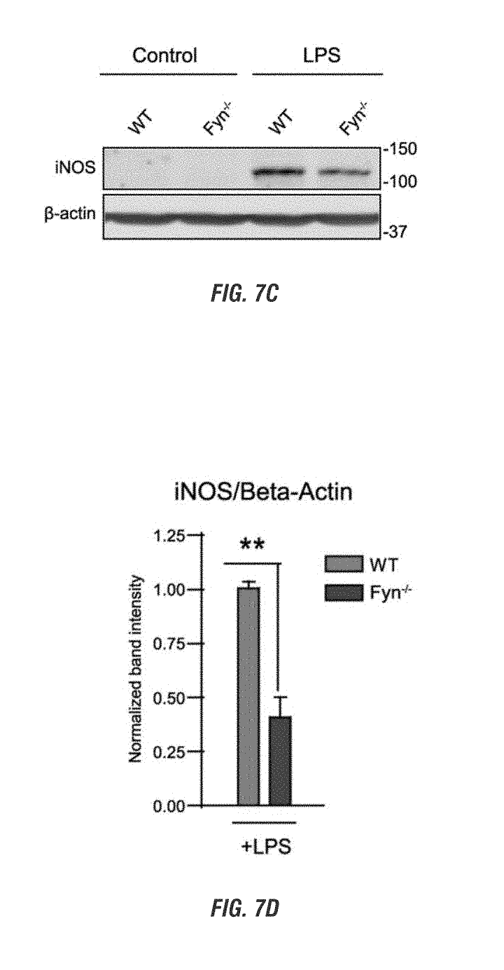

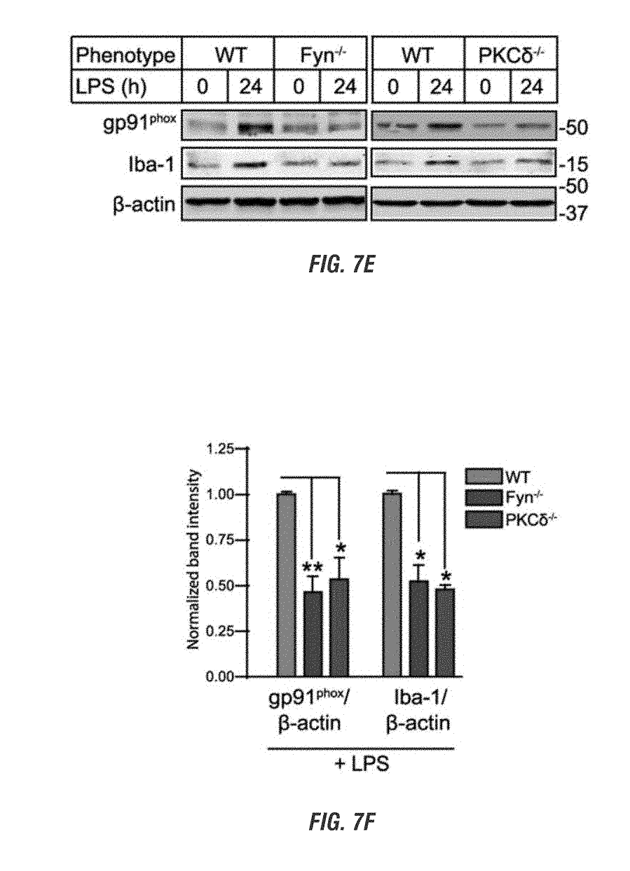

We further assessed whether Fyn alters the induction of iNOS and gp91^{phox}, which are key pro-inflammatory responses of microglial activation following LPS treatment. Treatment with LPS induced a stronger nitrite response from wild-type microglia than from Fyn^{-/-} microglia (FIG. 7A). This was further confirmed by immunostaining and immunoblotting for iNOS, the enzyme that mediates nitrite production. There was a greater induction of iNOS in Fyn wild-type microglia relative to Fyn^{-/-} microglia (FIGS. 7B-D). We also determined the expression of other key neuroinflammatory markers, including gp91^{phox} and Iba-1, in response to LPS stimulation. We, as well as other groups, have previously shown increased expression of the NADPH oxidase component gp91^{phox} and Iba-1 following LPS stimulation of primary microglia (Gao et al., 2011; Gordon et al., 2011). Western blot analysis revealed that LPS increased expression of both gp91^{phox} and Iba-1 in wild-type, but not in Fyn^{-/-} or PKCδ^{-/-} microglia (FIGS. 7E-F). Collectively, these data indicate that Fyn-PKCδ signaling plays a major pro-inflammatory role in microglial cells.

Fyn^{-/-} and PKCδ^{-/-} Mice are Resistant to LPS- and MPTP-Induced Neuroinflammatory Responses

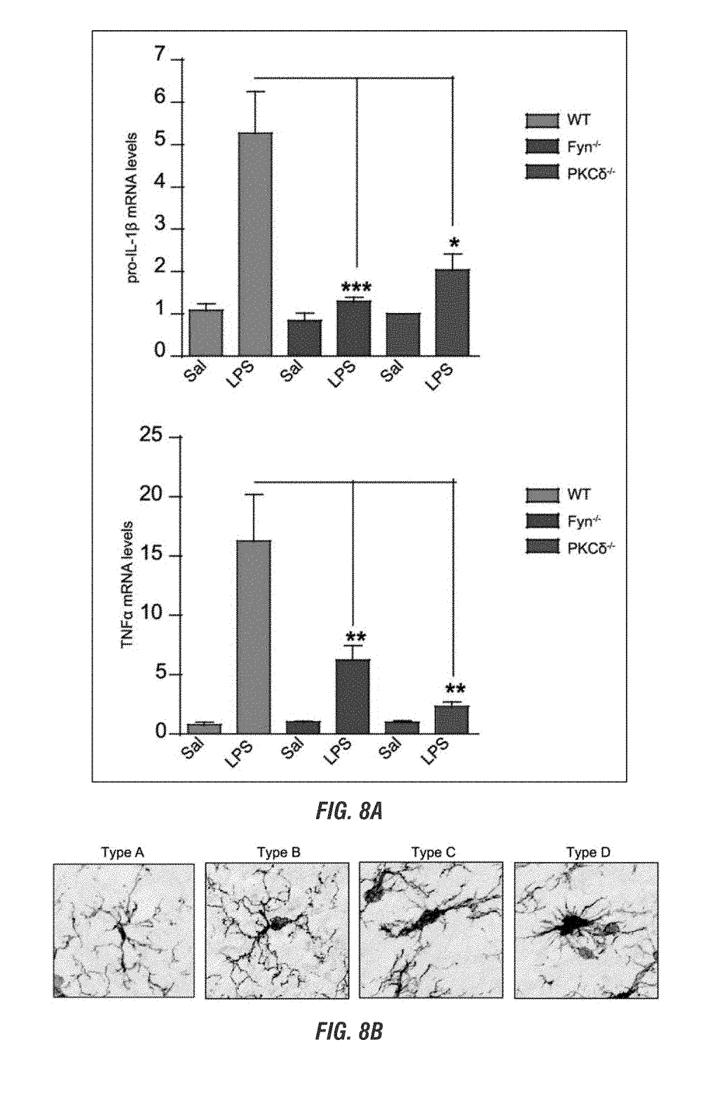

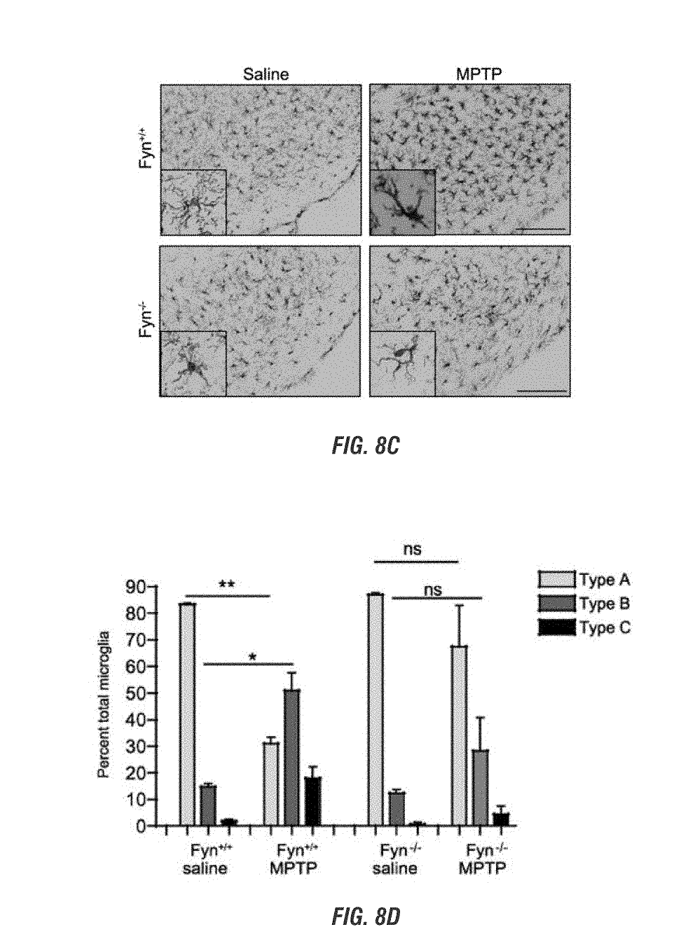

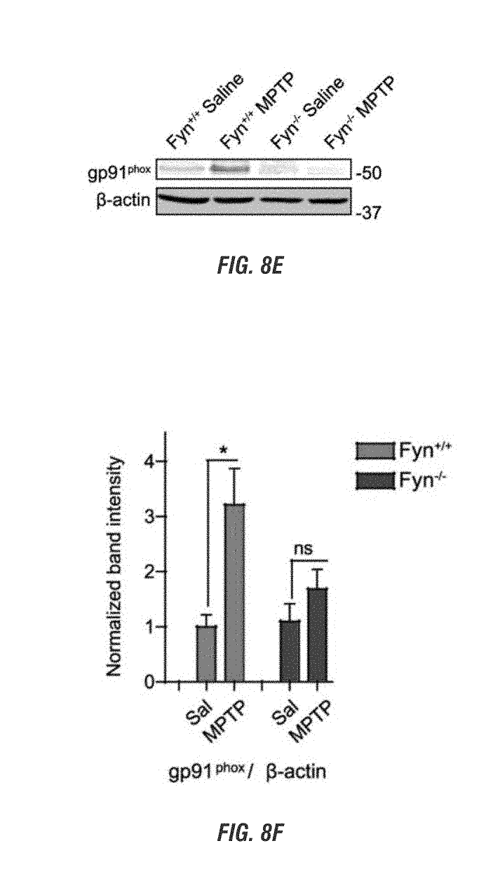

To extend our findings from isolated primary microglia to in vivo animal models of neuroinflammation, we first used the LPS model, which has previously been used to evoke neuroinflammatory responses in vivo (Choi et al., 2007; Qin et al., 2007). Wild-type (PKCδ^{+/+} and Fyn^{+/+}), PKCδ^{-/-} and Fyn^{-/-} mice were injected with 5 mg/kg LPS or PBS and were sacrificed 3 h later. Striatal mRNA contents of the pro-inflammatory cytokines pro-IL-1β and TNFα were determined by qRT-PCR. The levels of cytokine induction were almost identical in both wild-type groups, and we thus pooled the results. Systemic LPS administration strongly increased the levels of pro-IL-1β and TNFα transcripts in wild-type striata, but not in Fyn^{-/-} and PKCδ^{-/-} striata (FIG. 8A). To further establish the role of Fyn relevant to PD-associated neuroinflammation, we used the well-known Parkinsonian toxicant MPTP. We subjected wild-type and Fyn^{-/-} mice to an acute MPTP regimen (4×18 mg/kg, 2 h apart) and collected their brains for immunohistochemical analysis 24 h after the final MPTP injection. This acute MPTP model has been widely adopted for studying the neuroinflammatory response in the nigrostriatal pathway because maximal microglial activation occurs 24-48 h after the MPTP challenge (Wu et al., 2002; Wu et al., 2003; Sriram et al., 2006; Hirsch and Hunot, 2009). Following the MPTP challenge, successive 30-μm ventral midbrain sections from Fyn^{+/+} and Fyn^{-/-} mice were stained for the microglial marker Iba-1, and then microglial morphology was quantified using a recently well-established morphometric rating scale as discussed by others (Lastres-Becker et al., 2012). Representations of Type A-D microglial phenotype are provided in FIG. 8B. Treating Fyn^{+/+} mice with the acute MPTP regimen increased Iba-1 expression and discernibly shifted microglial morphology from its typical ramified state to its more amoeboid, activated morphology. After MPTP administration, significantly fewer Type A and more Type B and C microglia were observed in the Fyn^{+/+} SN, but this shift in microglial morphology was not apparent in the Fyn^{-/-} mice (FIGS. 8C-D). We also determined the induction of the NADPH oxidase component gp91^{phox} in MPTP animal model of neuroinflammation. Immunoblotting analysis revealed that MPTP increased expression of gp91^{phox} in WT but not in Fyn^{-/-} ventral midbrain tissues (FIGS. 8E-F). Overall, these results confirm that our in vitro data translate well to animal models of neuroinflammation.

Fyn^{-/-} and PKCδ^{-/-} Mice are Protected Against 6-OHDA-Induced Nigrostriatal Dopaminergic Neuronal Deficits and Microgliosis

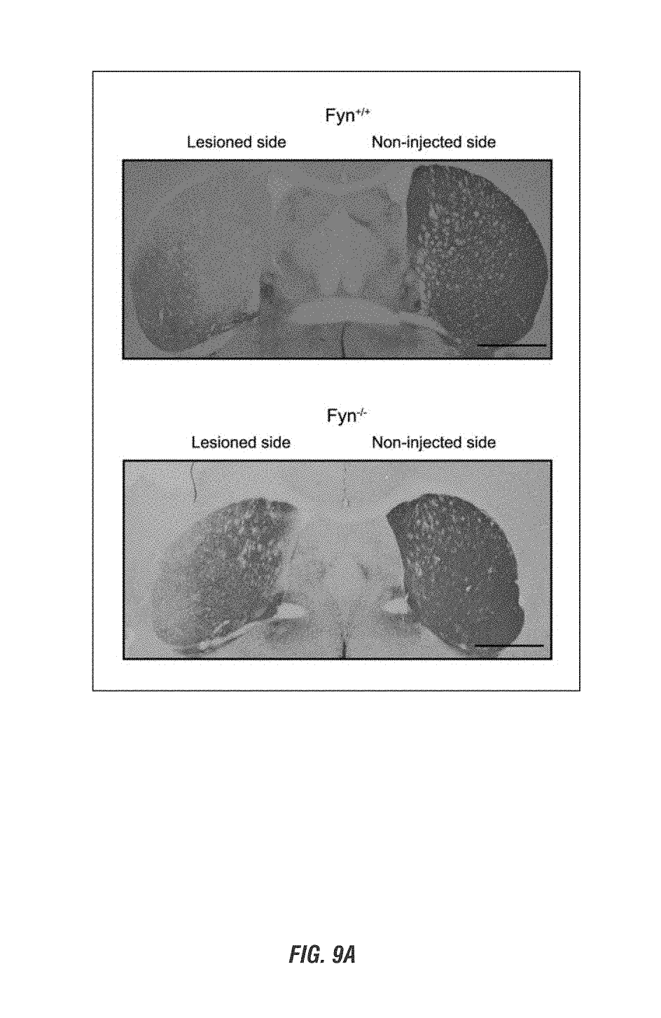

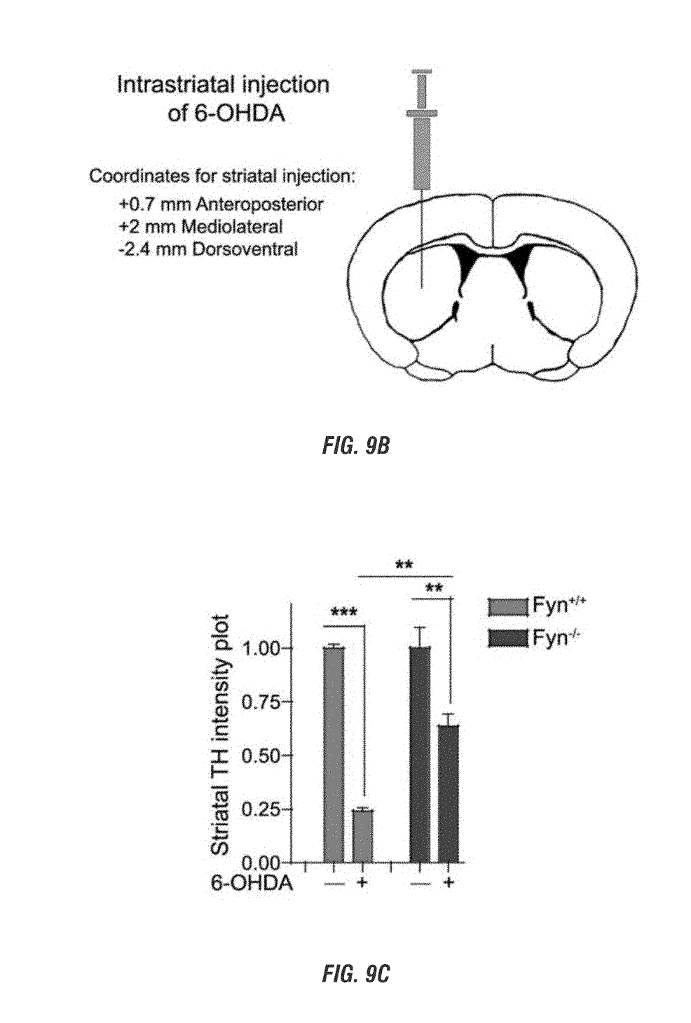

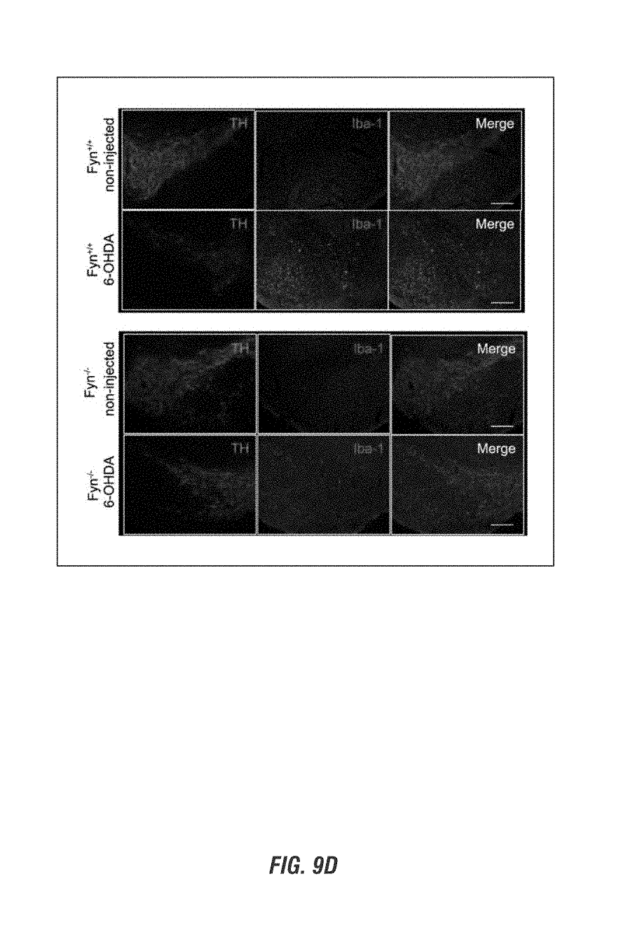

The 6-OHDA mouse model has recently been shown to elicit a neuroinflammatory response and neurodegeneration in the nigrostriatal dopaminergic system (Stott and Barker, 2014). While studying the role of Fyn in dopamine D1 receptor agonist-induced redistribution of NMDA receptor subunits, it was serendipitously discovered that Fyn^{-/-} mice were remarkably intransigent to 6-OHDA-induced behavioral deficits and striatal TH loss (Dunah et al., 2004). Fyn^{+/+} and Fyn^{-/-} mice, injected unilaterally with 6-OHDA (FIG. 9B) were sacrificed 9 days post-treatment, since mice at this treatment stage concurrently exhibit fewer striatal dopaminergic terminals, significantly fewer TH-positive cells in the SN, and microgliosis within the SN (Stott and Barker, 2014). Fyn^{-/-} mice were more resistant to 6-OHDA-induced striatal nerve terminal degeneration relative to Fyn^{+/+} mice (FIGS. 9A and C). We also show in our studies that 6-OHDA induced massive gliosis coupled with dopaminergic neuronal loss (FIG. 9D). However, Fyn^{-/-} mice show both greater survival of nigral dopaminergic neurons and a diminished neuroinflammatory microglial response.

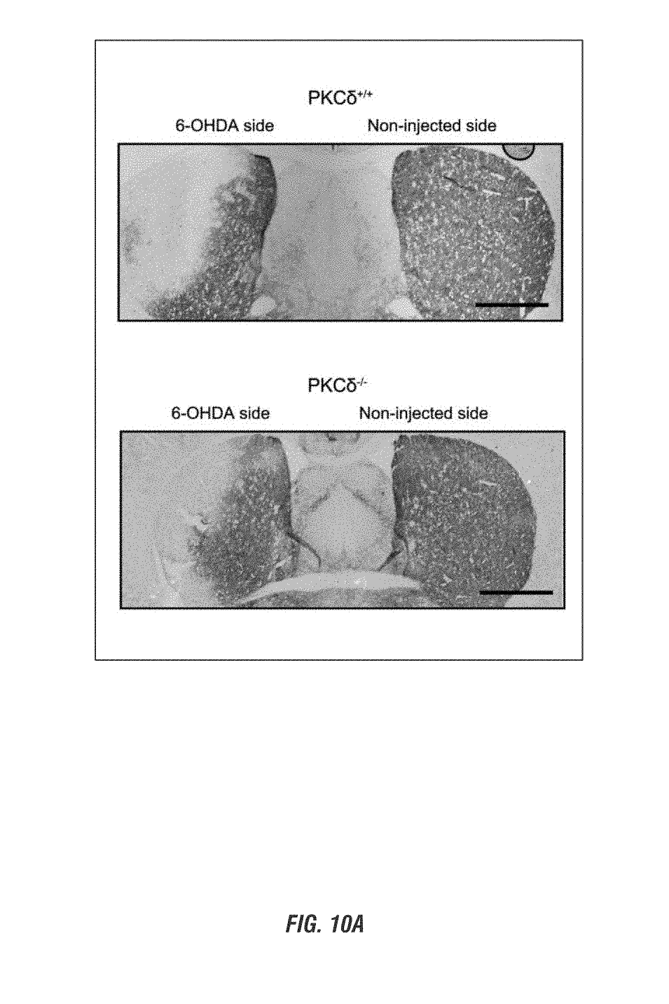

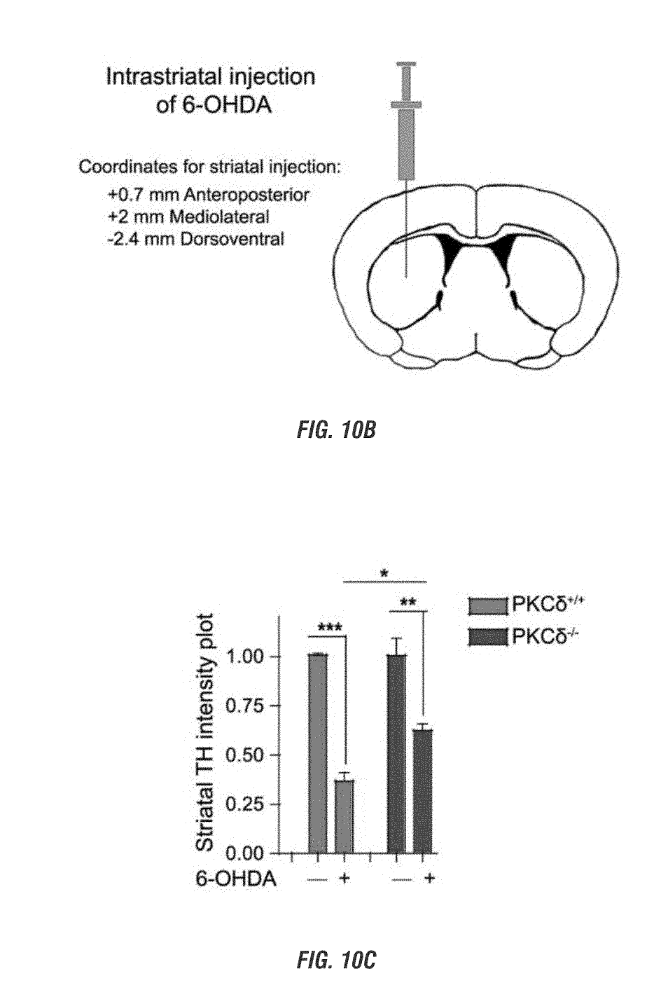

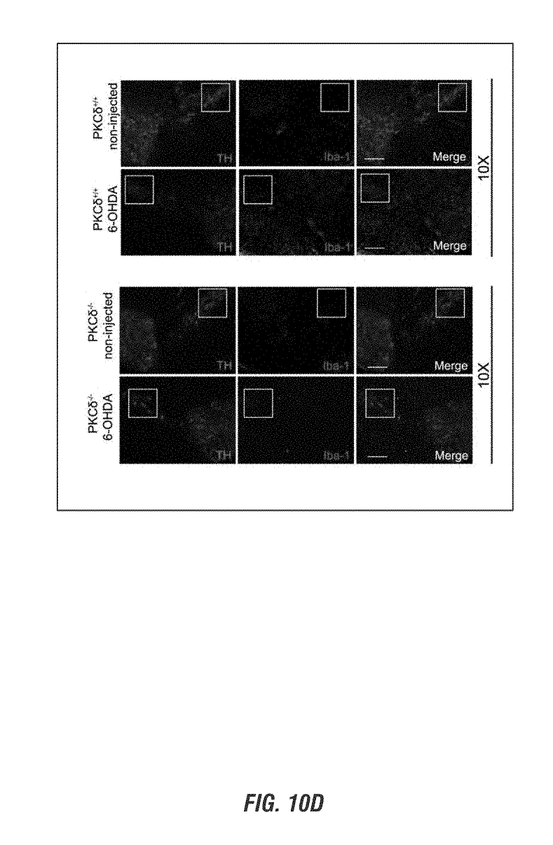

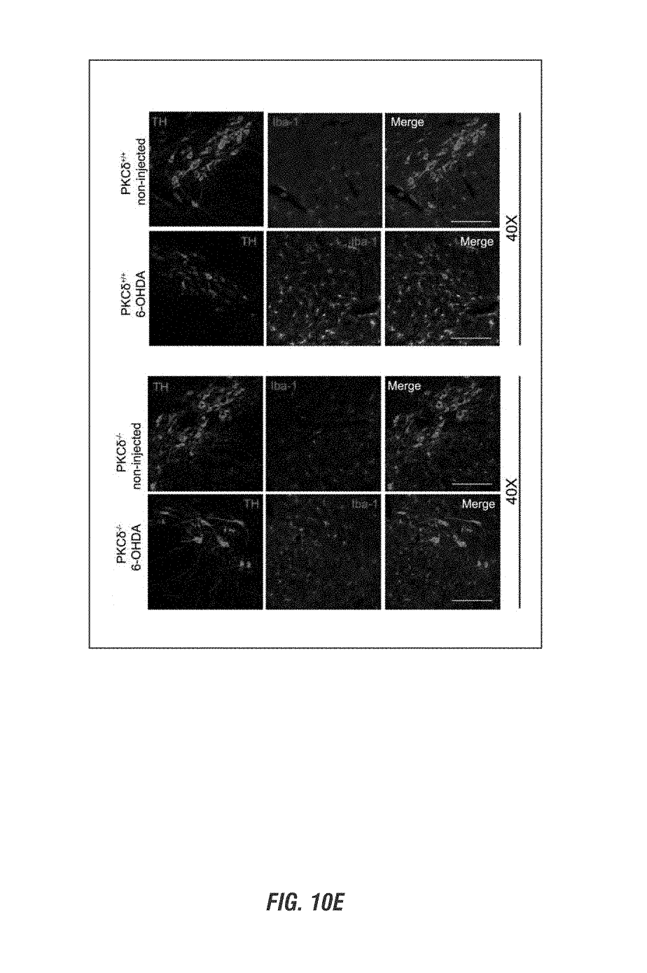

In the next set of in vivo experiments, we checked whether PKCδ^{-/-} mice were also resistant to 6-OHDA-induced nigral microgliosis and dopaminergic neuronal loss. PKCδ^{+/+} and PKCδ^{-/-} mice were injected unilaterally with 6-OHDA for 9 days, and DAB-TH immunostaining was performed on striatal sections as described above (FIG. 10B). Similar to Fyn^{-/-} mice, PKCδ^{-/-} mice showed reduced striatal TH loss following 6-OHDA treatment (FIGS. 10A and C). We also assessed nigral microgliosis by double-staining ventral midbrain sections for TH and Iba-1. As shown in FIG. 10D-E, PKCδ^{+/+} mice showed less TH-positive neuronal staining in the SN along with significantly more microgliosis on the ipsilateral side than on the contralateral side; however, the PKCδ^{-/-} mice showed a marked resistance to 6-OHDA-induced nigral TH loss as well as microgliosis. Thus, results from both Fyn and PKCδ knockout models of 6-OHDA neurotoxicity confirm the role of the Fyn-PKCδ signaling axis in a neuroinflammatory response in the nigrostriatal dopaminergic system.

### Diminished 6-OHDA-Induced Glial Neuronal Contact Formation in the Fyn^{-/-} Substantia Nigra

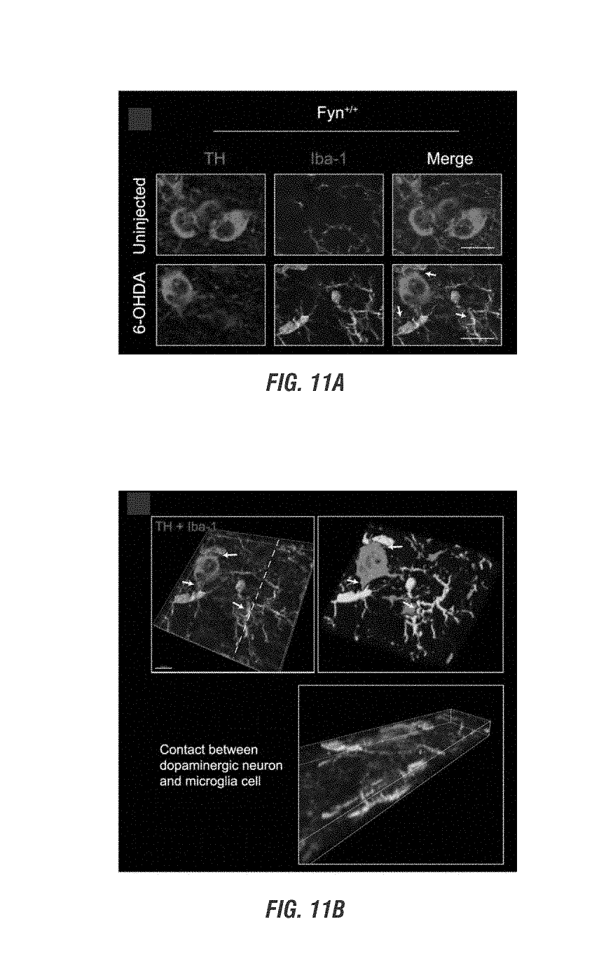

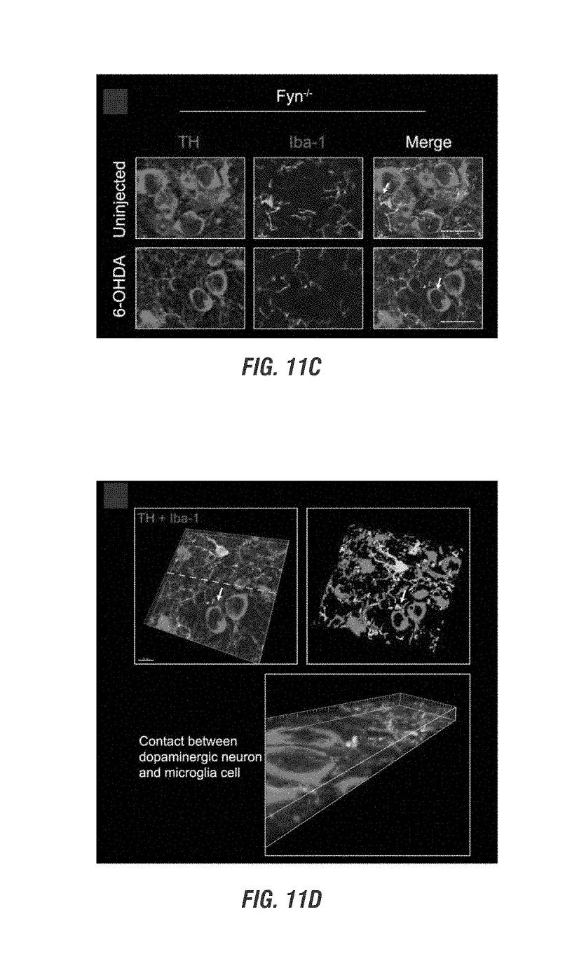

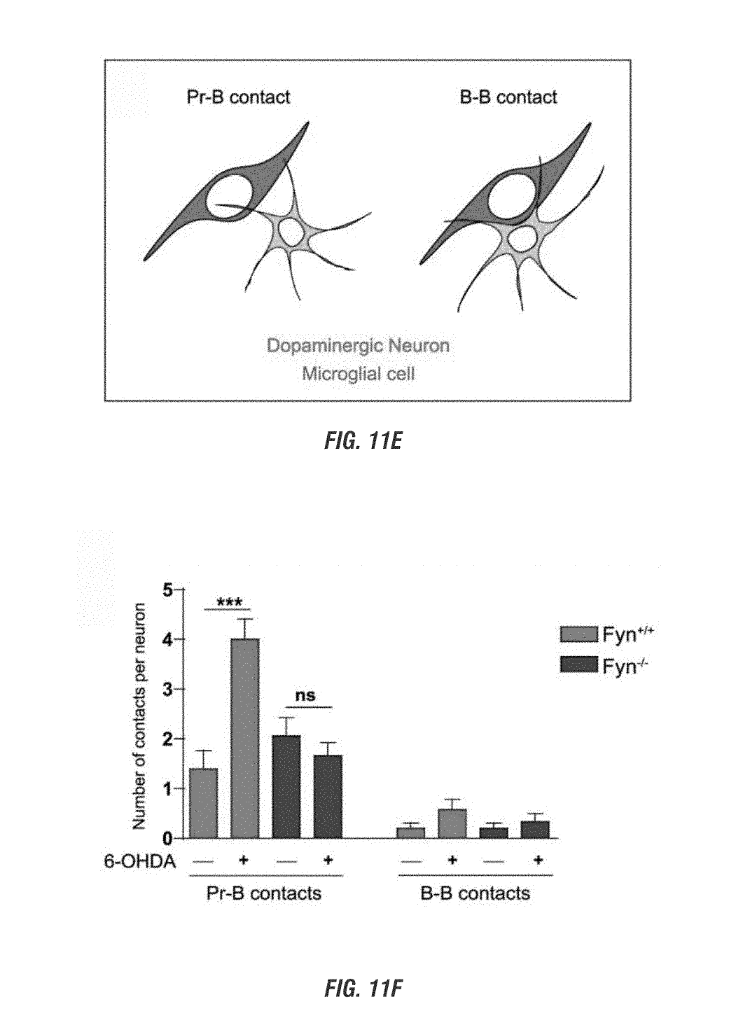

Recently, it was demonstrated that treating mice with MPTP rapidly increased the number of microglial-neuronal appositions, termed gliapses (Barcia et al., 2012). These contacts preceded neuronal phagocytosis by the microglia. Similar appositions between microglia and dopaminergic neurons were demonstrated in the 6-OHDA model, with evidence suggesting that microglial cells actually phagocytized neurons (Virgone-Carlotta et al., 2013), which has been postulated to occur if the neurons are dysfunctional. Our confocal high magnification Z stack image analysis (Imaris software) revealed a sharply increased number of microglial-neuronal contacts formed in the Fyn^{+/+} SN post-6-OHDA treatment as indicated by arrowheads (FIGS. 11A, B and F). The 3-D reconstructions of the respective stacks demonstrating contacts between dopaminergic neurons and microglia are shown adjacent to the original images (FIG. 11B). The number of gliapses per SN dopaminergic neuron was dramatically reduced in the 6-OHDA-injected Fyn^{-/-} mice (FIGS. 11C, D, and F). Typical contacts formed between microglial processes and dopaminergic neuronal cell bodies (termed Process-Body, or Pr-B contacts), and those formed between the microglial cell body and the dopaminergic neuronal cell body (Body-Body, or B-B contacts), are shown in FIG. 11E. Image analysis involving optical slices through the Z plane allowed us to both easily count gliapses and visualize actual engulfment events. Representative (FIGS. 11B and D) gliapses between a dopaminergic neuron and a microglial cell in the SN of 6-OHDA-injected Fyn^{+/+} and Fyn^{-/-} mice reveal a conspicuous reduction in the number of gliapses per neuron. Collectively, our confocal imaging results demonstrate Fyn plays a key role in activation of microglial morphological changes in vivo during inflammatory insults in nigrostriatal system.

### Prolonged Inflammogen Stimulation Effects Fyn Induction Upon Microglial Activation

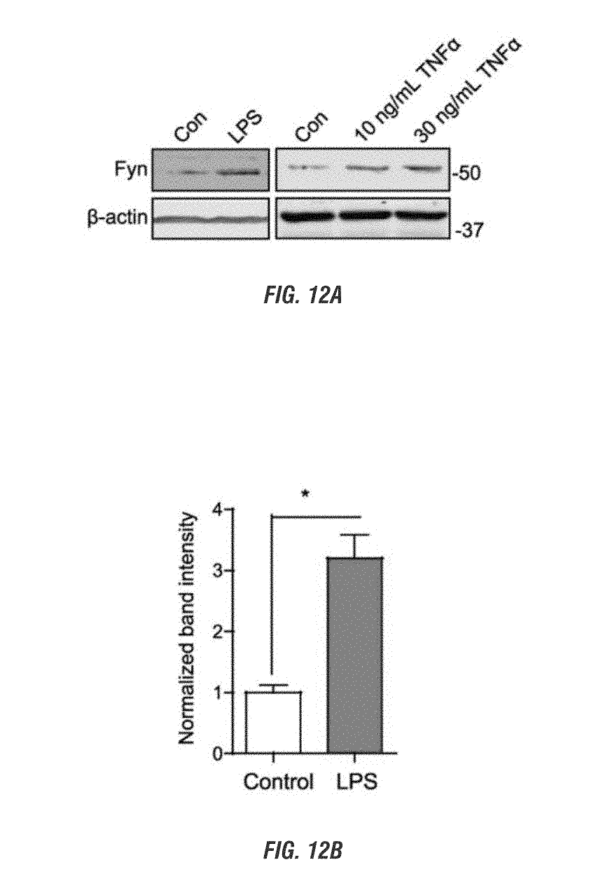

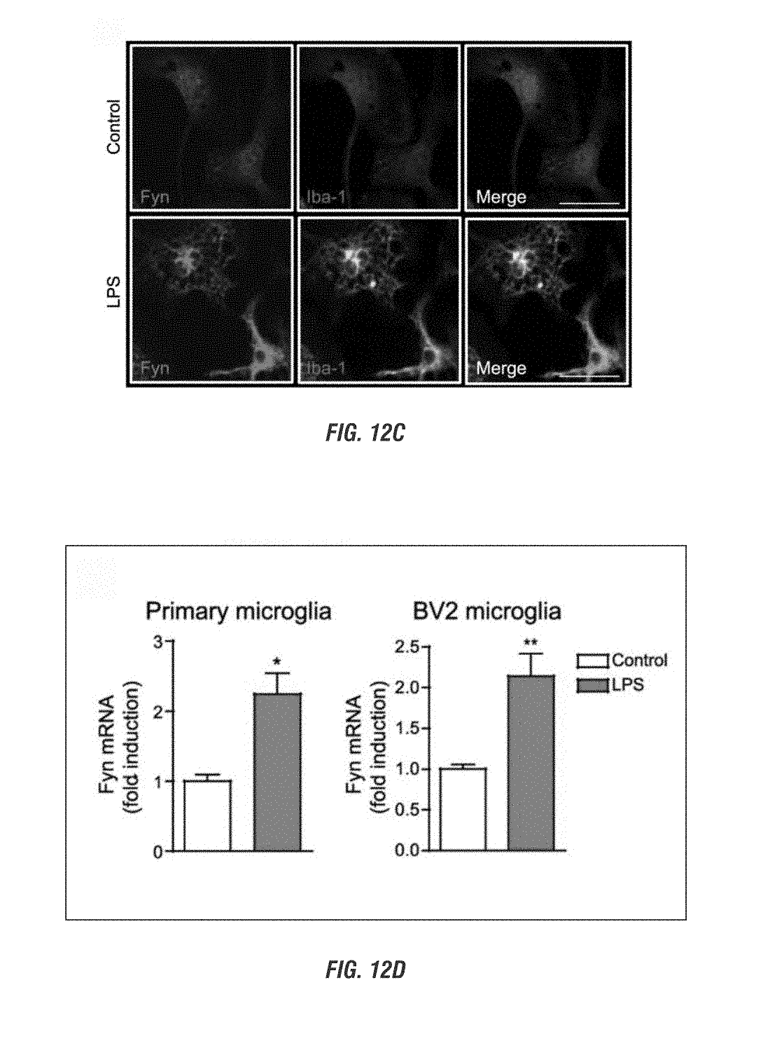

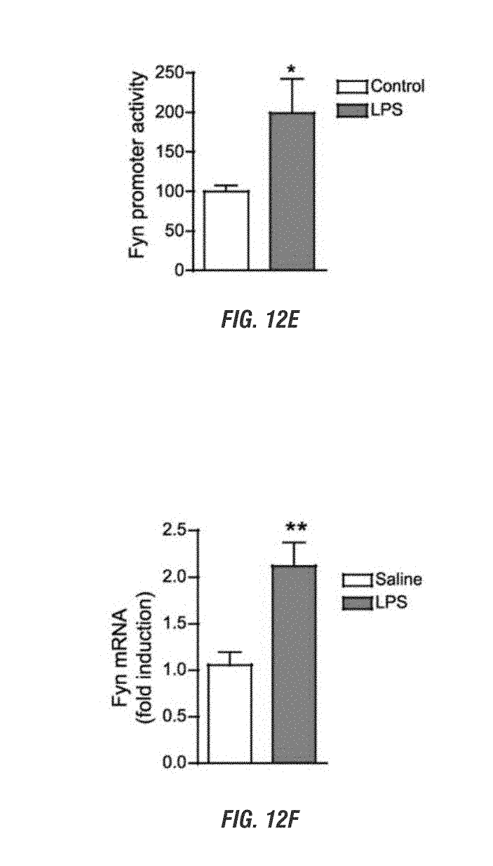

Thus far, our results demonstrated that short-term treatment of microglial cells with LPS and TNFα brings about an increase in Fyn activity, but not its expression. Strikingly, we discovered that prolonged treatment (12-24 h) of microglia with LPS or TNFα actually resulted in increased Fyn expression, evidenced by Western blot and immunocytochemistry (FIG. 12A-C). To confirm whether this is really due to induction of Fyn protein or increased protein stability, we performed qRT-PCR for Fyn mRNA expression in control and LPS-treated microglial cells. The result showed that treatment of microglia with LPS for 12 h brought about an increase in Fyn transcript levels (FIG. 12D). We also evaluated the effects of prolonged LPS treatment on Fyn promoter activity. For this, we transiently transfected primary microglia with a dual-luciferase Fyn reporter construct containing the 3.1 kb Fyn promoter fragment. LPS treatment significantly increased Fyn promoter activity (FIG. 12E), indicating strongly that Fyn is transcriptionally induced in microglial cells post-prolonged inflammogen administration. To further examine whether LPS upregulates Fyn mRNA expression, we injected wild-type mice with a single dose of LPS (5 mg/kg, i.p.) and evaluated the Fyn mRNA expression by qRT-PCR analysis. As shown in FIG. 12F, administration of LPS also induced Fyn transcript levels in the striatum. Together, these data suggest that prolonged LPS exposure induces Fyn gene upregulation in microglia, indicating that Fyn may have a sustained role in chronic neuroinflammatory processes.

### Discussion

Evidence from experimental models and human PD post-mortem studies strongly implicates the microglial-mediated inflammatory response as a major driver in the progression of PD; however, the key upstream cell signaling mechanisms that govern the neuroinflammatory processes have yet to be elucidated. Our results obtained from both cell culture and animal models provide novel insight into the role of the Fyn-PKCδ signaling cascade in regulating microglia-mediated neuroinflammation as related to PD pathogenesis. We have demonstrated dual regulation of pro-neuroinflammatory responses in microglia involving post-translational tyrosine phosphorylation of Fyn at its activation loop during the early stages of an inflammatory insult as well as transcriptional upregulation of Fyn upon prolonged exposure to pro-inflammatory stimuli. We have also showed that Fyn serves as a major upstream signaling molecule that works in concert with PKCδ to influence MAP kinase downstream and the NFκB pro-inflammatory cascade. Collectively, our study provides novel and significant insight into the pro-inflammatory function of Fyn-PKCδ signaling in PD models, and to the best of our knowledge, we are the first to discern this key signaling cascade that is relevant to microglia-mediated neuroinflammation in the nigrostriatal dopaminergic system.

We demonstrate that both the tyrosine kinase Fyn and the serine/threonine kinase PKCδ are differentially expressed in microglia and astrocytes (FIG. 1). No prior comparative data are available on Fyn and PKCδ expression in primary microglia. Although the roles of Src family kinases in TLR signaling are being identified, most studies have used peripheral immune and non-immune cells to determine Src kinase signaling. For example, multiple Src family kinases were activated by LPS in human lung microvascular endothelial cells (Gong et al., 2008). The activation of Src kinases mediated by TLR agonists depends on CD14, TLR2 and TLR4 (Reed-Geaghan et al., 2009), and Fyn has been shown to be associated with TLR2 in TLR2-overexpressing HEK293 cells (Finberg et al., 2012). Peritoneal macrophages have often been used as putative substitutes for brain microglia; Fyn contributes to CD36-mediated signaling responses upon Aβ₁₋₄₂ stimulation of macrophages (Moore et al., 2002). Of note, the authors reported unaltered LPS-induced MAP kinase activation in Fyn^{-/-} peritoneal macrophages when compared to WT macrophages. These apparent discrepancies may be attributed to the inherent differences between the microglial and macrophage gene expression profiles (Hickman et al., 2013). Many studies have used the p-Y416 Src family kinase antibody as a direct indicator of Fyn activation, without using immunoprecipitation or Fyn^{-/-} primary microglia as confirmatory tools to establish Fyn activation. In the present study, we demonstrate that Fyn is rapidly activated in primary microglia within 15-30 min of exposure to inflammogens (FIGS. 2A-F). Immunoprecipitation studies and experiments with Fyn^{-/-} microglia clearly confirmed that Fyn kinase is specifically activated during LPS and TNFα stimulation. LPS and TNFα activate microglia/macrophages via TLR4 and TNFα Receptor 1 (TNFR1) signaling, respectively (Olson and Miller, 2004; Parameswaran and Patial, 2010). Importantly, our study reveals that Fyn is a common signaling conduit in both TLR- and TNFR1-mediated signaling, since the TLR antagonist IAXO-101 and the TNFα signaling antagonist Etanercept attenuated Fyn activation (FIG. 2G). Immunocytochemistry analysis revealed that activated Fyn primarily localized to the microglial cell membrane. Although the functional relevance of this localization is not presently known, it is possible that movement of activated Fyn to the microglial membrane may regulate cell migration and cytokine release. Our results with the LPS mouse model provide in vivo evidence for rapid Fyn activation in the ventral midbrain region during inflammatory insults (FIG. 2I).

Our group has previously shown that PKCδ kinase proteolytic activation promotes oxidative stress-induced pro-apoptotic signaling pathways in dopaminergic neuronal cells (Kaul et al., 2003; Zhang et al., 2007; Jin et al., 2011a; Jin et al., 2011b). Recently, it was demonstrated that PKCδ is proteolytically cleaved by caspase-3 in LPS-treated BV2 cells (Burguillos et al., 2011). In the present study, we demonstrate that activated Fyn associates with PKCδ to phosphorylate the Y311 site, resulting in increased PKCδ kinase activity (FIG. 3). To the best of our knowledge, we are the first group to show the assembly of the Fyn-PKCδ signaling complex in microglial cells during pro-inflammatory conditions.

MAP kinase activation is necessary for cytokine production in various immune cell types, including microglia (El Benna et al., 1996; Koistinaho and Koistinaho, 2002). We demonstrate that Fyn-PKCδ signaling contributes to MAP kinase phosphorylation during microglial activation. Both LPS and TNFα stimulations rapidly activated the p38 and p-ERK MAP kinases in WT, but to a significantly lesser extent in the Fyn^{-/-} and PKCδ^{-/-} microglia (FIG. 4), indicating that Fyn-PKCδ signaling lies upstream of MAP kinase in microglia. Given that p38 is a prominent MAP kinase associated with the inflammatory cascade, our results suggest that Fyn and PKCδ are key upstream regulators of the pro-inflammatory function of this kinase. The downstream events of MAP kinase activation include NFκB signaling, which plays a cardinal role in eliciting pro-inflammatory responses in microglia. Selective inhibition of NFκB signaling has also proved beneficial in vitro as well as in an experimental mouse model of PD (Ghosh et al., 2007). We show here that IκBα degradation and p65-NFκB nuclear translocation were diminished in Fyn^{-/-} microglia stimulated with LPS or TNFα (FIG. 5), lending credence to the hypothesis that upstream Fyn signaling contributes to NFκB pathway activation in microglia. To our knowledge, the role of Fyn signaling in NFκB-mediated pro-inflammatory signaling in microglia has never been explored. Fyn has been shown to contribute to anaphylaxis inducer DNP₃₆-HSA mediated NFκB activation in Mast cells (Gomez et al., 2005b). More recently, Fyn was shown to mediate the nuclear translocation of p65-NFκB downstream of NKG2D and CD137 activation in natural killer cells, utilizing a signaling mechanism dependent on ADAP (Raj asekaran et al., 2013). This signaling pathway is almost certainly distinct from the Fyn-dependent microglial activation pathway, evidenced by the fact that ADAP^{-/-} microglia display unaltered pro-inflammatory responses (Engelmann et al., 2013).

Classical activation of microglia by TLR and TNFR1 agonists produces pro-inflammatory cytokines and chemokines, which mediate the downstream effects of microglial activation. Recently, we showed that TNFα directly induces dopaminergic neuronal apoptosis (Gordon et al., 2012). In our present study, the induction of the cytokines IL-6, IL-12 and TNFα was all diminished in Fyn^{-/-} and PKCδ^{-/-}_microglia in comparison to wild-type microglia (FIG. 6A). Consistently, genetic knockdown of Fyn via siRNA also resulted in diminished LPS-induced pro-inflammatory cytokine secretion (FIGS. 6B-C). TNFα-mediated production of IL-6 and TNFα was also diminished in Fyn deficient microglia (FIGS. 6D-E). Overexpressing the Fyn Y417A activation loop kinase deficient mutant construct in BV2 microglial cells also diminished LPS-stimulated cytokine production, implicating that the phosphorylation of tyrosine 417 is critical to pro-inflammatory function of Fyn (FIGS. 6F-G). Furthermore, we showed that the LPS-induced expression of iNOS and secretion of nitrite were significantly attenuated in the Fyn^{-/-} microglia (FIGS. 7A-D). We and several other groups have reported increased expression of the NADPH oxidase component gp91^{phox}, as well as the microglial marker Iba-1 following pro-inflammatory stimulation of microglia (Gao et al., 2011; Gordon et al., 2011). We demonstrate herein that prolonged LPS stimulation brought about the induction of these neuroinflammatory markers in wild-type, but not in Fyn^{-/-} and PKCδ^{-/-} microglia (FIGS. 7E-F).

We extended our in vitro studies to well-characterized animal models of neuroinflammation, wherein a single intraperitoneal injection of LPS in mice increases TNFα in the brain to levels that remain elevated long after serum TNFα levels have returned to normal (Qin et al., 2007). We utilized this model system to check for LPS-induced striatal pro-inflammatory cytokine induction in wild-type, Fyn^{-/-} and PKCδ^{-/-} mice. Strikingly, a single injection of LPS strongly increased WT striatal TNFα and pro-IL-1β mRNA levels; however, the induction of these cytokines was greatly diminished in Fyn^{-/-} and PKCδ^{-/-} striata (FIG. 8A). In addition to the LPS model, we also determined the pro-inflammatory role of Fyn in the well-studied acute MPTP model of neuroinflammation. MPTP induced reactive microgliosis and increased gp91^{phox} expression in the nigra of WT mice, but not in Fyn^{-/-} mice (FIGS. 8E-F). Interestingly, a quiescent ramified state of microglial morphology was observed in MPTP treated Fyn^{-/-} mice, while more amoeboid activated microglia were noted in Fyn wild-type mice (FIGS. 8C-D). In addition to the MPTP model, we further utilized the 6-OHDA-induced selective dopaminergic lesion model to validate that ablating Fyn or PKCδ confers resistance to nigrostriatal dopaminergic degeneration and microgliosis (FIGS. 9-10). Taken together, our results indicate that the Fyn-PKCδ signaling axis plays an important role in mediating pro-inflammatory response in both cell culture and animal models of neuroinflammation. Furthermore, our data indicate both Fyn and/or PKCδ can be exploited in the development of novel anti-neuroinflammatory drug candidates for treating PD and other related neurodegenerative diseases.

Recent imaging studies have demonstrated the formation of glial-neuronal contacts, called gliapses, formed between dopaminergic neurons and microglia that precede neuron loss in the MPTP model (Barcia et al., 2012; Barcia et al., 2013). To determine whether Fyn plays a role in microglial-dopaminergic neuron contact formation, we adopted the 6-OHDA mouse model. The formation of gliapses was described recently in the 6-OHDA model (Virgone-Carlotta et al., 2013). Our results from high magnification confocal analysis revealed the formation of gliapses was almost completely blocked in 6-OHDA injected Fyn^{-/-} mice (FIG. 11). The reduced number of gliapses correlated well with reduced dopaminergic neuronal loss following 6-OHDA administration to the Fyn^{-/-} mice. Lastly, prolonged stimulation of microglial cells with inflammogens strongly elicited an induction in Fyn kinase expression levels (FIG. 12). The aggregated form of α-synuclein, the primary component of PD-associated Lewy bodies, can activate microglia by utilizing CD36- and TLR2-dependent pathways (Su et al., 2008; Kim et al., 2013a). Studies are underway in our lab to demonstrate the role that Fyn plays in aggregated α-synuclein-induced neuroinflammatory events.

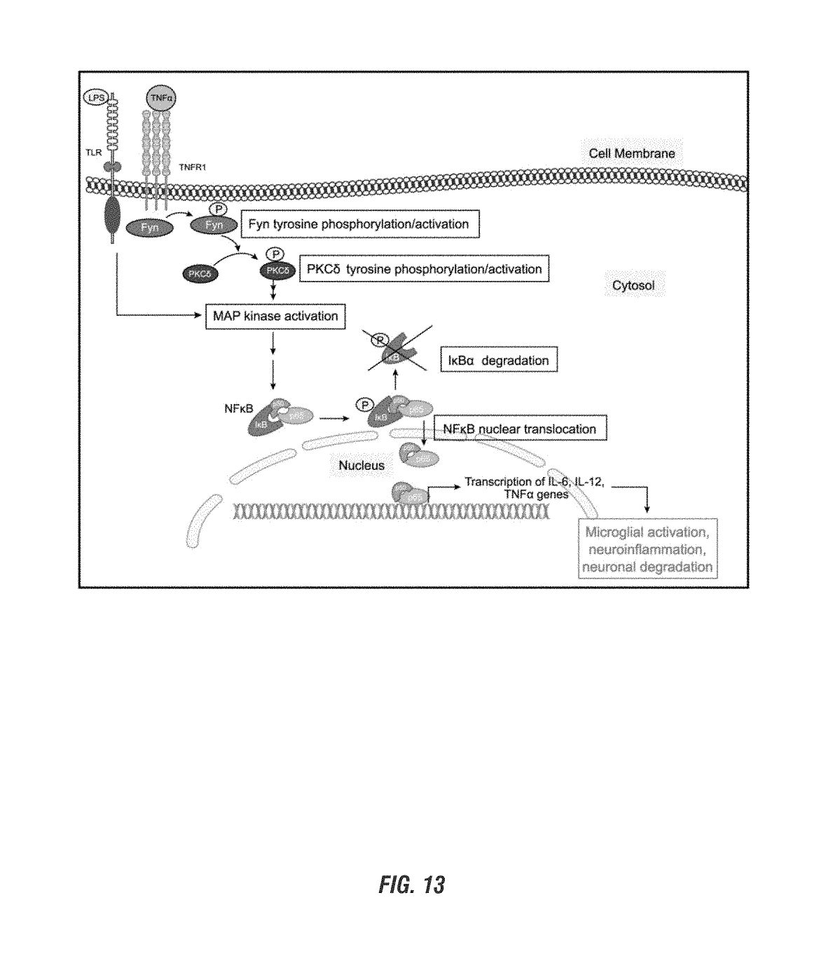

As summarized in FIG. 13, we demonstrate that Fyn activation plays an upstream regulatory role in eliciting pro-inflammatory signaling following both acute and chronic states of microglia stimulation. We arrived at this conclusion based on various lines of experimental evidence from cell culture, primary culture and in vivo models utilizing both Fyn and PKCδ knockout mice. Our mechanistic studies revealed that Fyn serves as a major upstream regulator of pro-inflammatory signaling involving PKCδ, MAP kinase and the NFκB pathways. Thus, Fyn could be exploited as a potential signaling node in the development of novel anti-neuroinflammatory drug candidates for treating PD and other related neurodegenerative diseases with associated microglia-mediated pro-inflammatory processes.

### Materials and Methods

### Chemicals and Reagents

Dulbecco's modified Eagle's medium/F-12 (DMEM/F-12), ascorbic acid, RPMI, fetal bovine serum (FBS), L-glutamine, Hoechst nuclear stain, penicillin, streptomycin and other cell culture reagents were purchased from Invitrogen (Gaithersburg, Md.). Recombinant TNFα was purchased from Peprotech (Rocky Hill, N.J.), and LPS (E. coli 0111:B4, Endotoxin content 6.6000000 EU/mg) and 6-OHDA were purchased from Sigma (St. Louis, Mo.). The mouse Fyn antibody was purchased from Thermo Scientific (Waltham, Mass.). Antibodies for rabbit Fyn, PKCδ, p-Y311 PKCδ, IκBα, Lamin-B, NOS2 (iNOS) and mouse Tubulin were purchased from Santa Cruz Biotechnology (Santa Cruz, Calif.). Antibodies against rabbit p-Src family kinase Y416 (p-Y416 SFK), native p65, p-p38 MAP kinase, native p38 MAP kinase, p-p44/42 MAP kinase (p-ERK) and native p44/42 MAP kinase (ERK) were purchased from Cell Signaling (Beverly, Mass.). The gp91^{phox} antibody was purchased from BD Biosciences (San Jose, Calif.). The mouse GFAP antibody was purchased from Millipore (Billerica, Mass.). The TH antibody was purchased from Chemicon (Temecula, Calif.). Mouse M2 FLAG and β-actin antibodies, as well as the rabbit β-actin antibody were purchased from Sigma. Rabbit and goat Iba-1 antibodies were purchased from Wako Chemicals (Richmond, Va.) and Abcam (Cambridge, Mass.), respectively. The goat TNFα antibody was purchased from R&D Systems (Minneapolis, Minn.). ³²P-ATP was purchased from Perkin Elmer (Boston, Mass.) and the histone substrate from Sigma. The Bradford protein assay kit was purchased from Bio-Rad Laboratories (Hercules, Calif.). FLAG-tagged human WT Fyn and Y417A mutant Fyn constructs were obtained as described previously (Kaspar and Jaiswal, 2011).

### Animal Studies

The Fyn^{-/-} and PKCδ^{-/-} mice used in these studies were bred in our animal facility. Fyn^{-/-} mice were originally obtained from Dr. Dorit Ron's laboratory at the University of California, San Francisco and are available from Jackson Laboratory (stock number 002271). PKCδ^{-/-} mice were obtained originally from Dr. Keiichi Nakayama's laboratory (Division of Cell Biology, Department of Molecular and Cellular Biology, Medical Institute of Bioregulation, Kyushu University, Fukuoka, Japan). Wild-type (Fyn^{+/+} and PKCδ^{+/+}), PKCδ^{-/-} and Fyn^{-/-} mice were housed under standard conditions of constant temperature (22±1° C.), humidity (relative, 30%), and a 12-h light cycle with food and water provided ad libitum. Six- to eight-week-old male mice were used for all studies. The well-characterized acute MPTP mouse model of PD (Wu et al., 2003; Przedborski et al., 2004; Kim et al., 2007; Hu et al., 2008) was primarily used for neuroinflammation studies. The mice from the MPTP treatment group received 4 intraperitoneal (i.p.) injections of MPTP-HCl (18 mg/kg free-base) dissolved in saline at 2-h intervals. Mice were sacrificed 24 h after the last injection. The nigral neuroinflammatory response was also studied using the systemic LPS injection model (Qin et al., 2007), which induces chronic neuroinflammation and progressive dopaminergic degeneration in mice. A single injection of LPS (5 mg/kg, i.p.) was delivered to wild-type, Fyn^{-/-} and PKCδ^{-/-} mice. Mice were sacrificed 24 to 48 h later. Control groups for both MPTP and LPS received equivolume injections of saline. We injected 2 μL of 6-OHDA, diluted at a concentration of 5 μg/μL in 0.02% ascorbic acid, into the left striatum (0.2 μL/min) using the Angle 2 stereotaxic apparatus (Leica Biosystems, St. Louis, Mo.). The coordinates, relative to bregma were: 0.7 mm anteroposterior, 2 mm lateral, and 2.4 mm ventral. The contralateral side was either not injected or injected with 2 μL of 0.02% ascorbic acid diluted in sterile PBS as a negative control. All animal procedures were approved by the Iowa State University Institutional Animal Care and Use Committee (IACUC).

### Primary Microglial Cultures and Treatments

Primary microglial cultures were prepared from wild-type, Fyn^{-/-} and PKCδ^{-/-} postnatal day 1 (P1) mouse pups as described previously (Gordon et al., 2011). Briefly, mouse brains were harvested, meninges removed, and then placed in DMEM-F12 supplemented with 10% heat-inactivated FBS, 50 U/mL penicillin, 50 μg/mL streptomycin, 2 mM L-glutamine, 100 μM non-essential amino acids, and 2 mM sodium pyruvate. Brain tissues were then incubated in 0.25% Trypsin-EDTA for 30 min with gentle agitation. The trypsin reaction was stopped by adding double the volume of DMEM/F12 complete medium and then washing brain tissues three times. Tissues were then triturated gently to prepare a single cell suspension, which was then passed through a 70-μm nylon mesh cell strainer to remove tissue debris and aggregates. The cell suspension was then made up in DMEM/F12 complete medium and seeded into T-75 flasks, which were incubated in humidified 5% CO₂ at 37° C. The medium was changed after five to six days and the mixed glial cells were grown to confluence. Microglial cells were separated from confluent mixed glial cultures by differential adherence and magnetic separation to >97% purity, and then were allowed to recover for 48 h after plating. Primary microglia were treated in DMEM/F12 complete medium containing 2% FBS. For signaling experiments, the protocol employed by Stuart (2007) was utilized with a small modification. For this, the primary microglial cells were kept in 2% DMEM/F12 complete medium for 5 h at 37° C. prior to treatment. The microglial cells were treated with 100-200 ng/mL LPS and 10-30 ng/mL TNFα for durations sampled at pre-specified time points. We selected the LPS doses used in this study based on previous studies in which stimulation of cultured primary mouse microglia with 100 and 200 ng/ml LPS resulted in significant microglial activation (Haynes et al., 2006; Crotti et al., 2014; Lee et al., 2014).

siRNAs and Transfections of Microglia

Transient transfections of primary microglia with Fyn promoter reporter were performed using Lipofectamine LTX & Plus Reagent according to the manufacturer's protocol. Primary microglia were plated at 0.75×10⁶ cells/well in 12-well plates one day before transfection. We transiently transfected 3 μg of Fyn promoter construct. Cells were treated 24 h after transfection with or without 200 ng/mL of LPS for 12 h and then lysed. Luciferase activity was measured using a Dual-luciferase assay kit (Promega) on a Synergy 2 multi-mode microplate reader (BioTek). Firefly luciferase luminescence values were used to normalize Renilla luciferase luminescence values. The pre-designed, on-target plus SMART pool Fyn siRNA (a combination of four siRNAs, Cat. No. LQ-040112-00-0002) and scrambled siRNA (Cat. No. D-001210-03-05) were purchased from Dharmacon (Lafayette, Colo.). We carried out siRNA transfections in primary mouse microglial cells with Lipofectamine 3000 reagent according to the manufacturer's protocol. Briefly, primary microglia were plated at 2×10⁶ cells/well in 6-well plates one day before transfection. For each well, 300 pmol of Fyn siRNA pool (75 pmol each) or an equal amount of scrambled siRNA mixed with 5 μl of Lipofectamine 3000 were added to the cells. Seventy-two hours after the initial transfection, cells were analyzed by Western blotting to confirm the extent of Fyn knockdown or treated with LPS (200 ng/mL) for 24 further hours, after which cytokine content was analyzed by Luminex bioassay.

Transfection of BV2 microglia with WT Fyn-FLAG, Y417A Fyn-FLAG and Empty Vector pcDNA3.1 constructs was performed by using the AMAXA Nucleofector Kit. Briefly, BV2 cells were resuspended in transfection buffer (Solution 1: 400 μM ATP-disodium (Sigma A7699), 600 μM MgCl₂-6H₂O in water; Solution 2: 100 μM KH₂PO₄, 20 μM NaHCO₃, 5 μM glucose in water) to a final concentration of 3×10⁶ cells per 100 μl and mixed with the respective vector; 5 μg of vector DNA was used per transfection.

### Immunohistochemistry and Immunofluorescence Studies

Immunohistochemistry was performed on sections from the substantia nigra and other brain regions of interest as described previously (Jin et al., 2011b; Ghosh et al., 2013). Briefly, mice were anesthetized with a mixture of 100 mg/kg ketamine and 10 mg/kg xylazine and then perfused transcardially with freshly prepared 4% paraformaldehyde (PFA). Extracted brains were post-fixed in 4% PFA for 48 h and 30-μm sections were cut using a freezing microtome (Leica Microsystems). Antigen retrieval was performed in citrate buffer (10 mM sodium citrate, pH 8.5) for 30 min at 90° C. Sections were then washed several times in PBS and blocked with PBS containing 2% BSA, 0.2% Triton X-100 and 0.05% Tween 20 for 1 h at room temperature. Sections were then incubated with primary antibodies overnight at 4° C. and washed 7 times in PBS on a Belly Dancer Shaker (SPI supplies). The sections were incubated with Alexa dye-conjugated secondary antibodies for 75 min at room temperature and their cell nuclei were stained with Hoechst dye. Sections were mounted on slides using Prolong antifade gold mounting medium (Invitrogen) according to the manufacturer's instructions. Samples were visualized using an inverted fluorescence microscope (Nikon TE-2000U) and images were captured using a Spot digital camera (Diagnostic Instruments Inc).

Immunofluorescence studies in primary microglia were performed according to previously published protocols with some modifications (Gordon et al., 2011). Briefly, microglial cells were grown on poly-D-lysine-coated coverslips and treated 48 h later. At the end of treatments, cells were fixed with 4% PFA, washed in PBS and incubated in blocking buffer (PBS containing 2% BSA, 0.5% Triton X-100 and 0.05% Tween 20) for 1 h at room temperature. The coverslips were then incubated overnight at 4° C. with respective primary antibodies diluted in PBS containing 2% BSA. Samples were then washed several times in PBS and incubated with Alexa 488 and 555 dye-conjugated secondary antibodies. The nuclei were labeled with Hoechst stain (10 μg/mL) and coverslips were mounted with Fluoromount medium (Sigma Aldrich) on glass slides for visualization. Quantification of the number of microglial/astroglial cells obtained post-separation was accomplished using JACoP, a downloadable ImageJ plugin from Fabrice P. Cordelieres. Original Hoechst or antibody TIFF files were converted into 8-bit black-and-white images, and a colocalization image was generated. Counting of Hoechst-positive and Iba-1+Hoechst-positive cells was done using the Cell counter function of the default ‘Analyze’ plugin in ImageJ.

### Confocal Imaging and Z Stack Image Acquisition and Analysis

Confocal imaging was performed at the Iowa State University Microscopy Facility, using a Leica DMIRE2 confocal microscope with the 63× and 43× oil objectives and Leica Confocal Software. One optical series covered 11-13 optical slices of 0.5-μm thickness each. Microglial neuronal contact identification and quantification were performed by counting the number of colocalizations of the two markers, with TH marked red by anti 555 and Iba-1 marked green by anti 488 using the methodology described by Barcia and colleagues (Barcia et al., 2012). The Imaris software was used to analyze the Z stack images for contact identification. The surface reconstruction wizard in the Imaris software was used to make 3-D reconstructions of stacks for easier viewing of microglial-dopaminergic contacts and surface topology.

qRT-PCR

RNA isolation from primary microglial cells and brain tissue samples was performed using the Absolutely RNA Miniprep Kit, and then 1 μg total of isolated RNA was used for reverse transcription with the AffinityScript qPCR cDNA synthesis system (Agilent Technologies) according to the manufacturer's instructions. Quantitative SYBR Green PCR assays for gene expression were performed using the RT² SYBR Green Master Mix with pre-validated primers (SABiosciences qPCR assay system). Catalog numbers of the primers were Fyn—PPM04015A, pro-IL1β—PPM03109E, TNFα—PPM03113G. The mouse 18S rRNA gene (catalog number—PPM57735E) was used as the housekeeping gene for normalization. For each primer, the amount of template providing maximum efficiency without inhibiting the PCR reaction was determined during initial optimization experiments. For all experiments, dissociation curves were generated to ensure a single peak was obtained at the right melting temperature without non-specific amplicons. The fold change in gene expression was determined by the ΔΔCₜ method using the threshold cycle (Cₜ) value for the housekeeping gene and the respective target gene of interest in each sample.

### Western Blotting

Brain tissue and microglial cell lysates were prepared using modified RIPA buffer and were normalized for equal amounts of protein using the Bradford protein assay kit. Equal amounts of protein (12 to 25 μg for cell lysates and 30-40 μg for tissue lysates) were loaded for each sample and separated on either 12% or 15% SDS-PAGE gels depending on the molecular weight of the target protein. After separation, proteins were transferred to a nitrocellulose membrane and the nonspecific binding sites were blocked for 1 h using a blocking buffer specifically formulated for fluorescent Western blotting (Rockland Immunochemicals). Membranes were then probed with the respective primary antibodies for 3 h at room temperature or overnight at 4° C. After incubation, the membranes were washed 7 times with PBS containing 0.05% Tween 20, and then Secondary IR-680-conjugated anti-mouse (1:10,000, goat anti-mouse, Molecular Probes) and IR-800 conjugated anti rabbit (1:10,000, goat anti-rabbit, Rockland) were used for antibody detection with the Odyssey IR imaging system (LiCor). Membranes were visualized on the Odyssey infrared imaging system. Antibodies for β-actin and Tubulin were used as loading controls.

### Co-Immunoprecipitation Studies

We adopted an immunoprecipitation (IP) protocol with slight modifications from Gao and colleagues (Gao et al., 2011). Cell lysates were prepared in TNE buffer (10 mM Tris-HCl at pH 7.5, 1% Nonidet P-40, 0.15 M NaCl, 1 mM EDTA, and 1:100 protease inhibitor cocktail) and centrifuged at 17,400×g for 40 min at 4° C. The supernatant protein concentration was measured and normalized between samples. Approximately 50 μL of the sample containing 20 μg protein was used as input. For immunoprecipitation analysis, 1 mg of protein in 400 μL TNE buffer was used. Next, 10 μL (2 μg) of Fyn rabbit polyclonal antibody was added to the lysates, and the samples were set on an orbital shaker overnight at 4° C. The next day, protein G Sepharose beads were spun down at 17,400×g for 5 min and the ethanol supernatant was replaced with an equal volume of the lysis buffer. The Protein G Sepharose slurry was washed once and 50 μL was added to each sample. The samples were set on an orbital shaker overnight at 4° C. Protein G beads were collected by centrifugation at 2000×g for 5 min and were washed four times with TNE buffer. The bound proteins were eluted by boiling in 2× protein-loading dye for 5 min. Immunoblots were performed on 12% SDS-PAGE gels as described for Western blotting.

### Nuclear and Cytoplasmic Fractionation

Nuclear and cytoplasmic fractions were performed using the NE-PER Kit (Thermo Scientific) as previously described (Jin et al., 2011a; Jin et al., 2014). Briefly, 5×10⁶ cells were treated with LPS or TNFα for 15 min. CER1 reagent (200 μL) was used for each sample to extract the cytoplasmic fraction, and 50 μL of NER reagent was used to extract the nuclear fraction. Tubulin or β-actin was used as a cytosolic fraction marker. Lamin B was used as a nuclear fraction marker.

### Fyn Kinase Assays

Cell pellets were washed with ice-cold PBS and resuspended in lysis buffer (25 mM HEPES at pH 7.5, 20 mM β-glycerophosphate, 0.1 mM sodium orthovanadate, 0.1% Triton X-100, 0.3 M NaCl, 1.5 mM MgCl₂, 0.2 mM EDTA, 0.5 mM dithiothreitol, 10 mM NaF, and 4 μg/mL each of aprotinin and leupeptin) (Kaul et al., 2005). Next, 50 μg of crude protein was incubated with 150 mM Fyn kinase substrate (Biomol), 100 mCi of [γ-³²P] ATP, Src-Mn-ATP cocktail and Src reaction buffer (Millipore) for 10 min at 30° C. with agitation. To precipitate the Fyn kinase substrate peptide, 20 mL of 40% trichloroacetic acid was added. Then 25 μL of the mixture was spotted onto a P81 phosphocellulose square, and 5 min after spotting, the squares were washed five times in 0.75% phosphoric acid in PBS with a final wash step in acetone. The squares were transferred into a scintillation vial and the CPMA counts were read in a liquid scintillation system after adding 5 mL of scintillation cocktail to each vial.

### PKCδ Kinase Assays

PKCδ IP kinase activity assays were performed as described previously (Anantharam et al., 2002; Latchoumycandane et al., 2011; Harischandra et al., 2014) with some modifications for microglial cells. Briefly, primary microglial cells were collected after treatments, washed in ice-cold PBS and resuspended in a mild RIPA lysis buffer containing protease and phosphatase inhibitor cocktail (Pierce Biotechnology). The cells were placed on ice for 20 min to allow for complete lysis and then centrifuged at 16,200×g for 45 min. The supernatant protein concentration was determined using the Bradford protein assay kit. Samples were normalized to a uniform total protein concentration of 2 μg/mL, and then 200 μg of total protein in a 250 μL reaction volume was immunoprecipitated overnight at 4° C. using 5 μg of PKCδ antibody. The next day, protein-A agarose beads (Sigma-Aldrich) were incubated for 1 h at room temperature. The protein A-bound antibody complexes were collected and washed 3 times in 2× kinase assay buffer (40 mM Tris, pH 7.4, 20 mM MgCl₂, 20 μM ATP, and 2.5 mM CaCl₂), and then resuspended in the same buffer. The kinase reaction was started by adding 40 μL of the reaction buffer containing 0.4 mg of histone H1, 50 μg/mL phosphatidylserine, 4 μM dioleoylglycerol, and 10 μCi of [γ-³²P] ATP at 3000 Ci/mM to the immunoprecipitated samples. The samples were then incubated for 10 min at 30° C. and the kinase reaction was stopped by adding 2×SDS loading buffer and boiling for 5 min. Proteins were separated on a 15% SDS-PAGE gel and the phosphorylated histone bands were imaged using a Fujifilm FLA 5000 imager. Image analysis and band quantification were performed using ImageJ.

### Nitric Oxide Detection

Nitric oxide production by primary microglia was measured indirectly by quantification of nitrite in the supernatant using the Griess reagent (Sigma Aldrich). Microglia were plated in poly-D-lysine-coated 96-well plates at 1×10⁵ cells/well. Cells were treated with 100 ng/mL of LPS for 24 h and after 100 μL of supernatant was collected from each well, an equal volume of the Griess reagent was added. The samples were incubated on a plate shaker at room temperature for 15 min until a stable color was obtained. The absorbance at 540 nm was measured using a Synergy 2 multi-mode microplate reader (BioTek Instruments) and the nitrite concentration was determined from a sodium nitrite standard curve.

### Multiplex Cytokine Luminex Immunoassays

Primary microglia obtained from wild-type, PKCδ^{-/-} and Fyn^{-/-} mice were seeded in poly-D-lysine-coated 96-well plates at 1×10⁵ cells/well. The cells were treated for 24 h with 100-200 ng/mL LPS or 10 ng/mL TNFα. After treatment, 50 μL of supernatant from each well was collected and frozen at −80° C. The levels of cytokines and chemokines in the supernatants were determined using the Luminex bead-based immunoassay platform (Vignali 2000) and pre-validated multiplex kits (Milliplex mouse cytokine panel Millipore) according to the manufacturer's instructions.

### DAB Immunostaining and Grading of Microglial Morphology

Iba-1 diaminobenzidine (DAB) immunostaining was performed on striatal and substantia nigral sections as described previously (Ghosh et al., 2010). Briefly, mice were perfused with 4% PFA and brains were post-fixed with PFA for 48 h before storage in 30% sucrose. Fixed brains were embedded in O.C.T compound (Tissue-Tek) and stored frozen at −80° C. until the frozen blocks were sliced into 30-μm coronal sections using a cryostat. Sections were probed with the primary antibodies overnight at 4° C. and then incubated with biotinylated anti-rabbit secondary antibody. The sections were then treated with Avidin peroxidase (Vectastain ABC Elite kit). The DAB reagent was used for producing the brown colored stain. Grading of microglial morphology was performed as described elsewhere (Lastres-Becker et al., 2012). For microglial grading, images were sharpened in ImageJ so the morphology could be more clearly visualized. The cell counter function in the ‘Analyze’ plugin was used to count the number of Type A, B, C and D microglia in the ventral midbrain sections.

### Data Analysis

Data analysis was performed using Prism 4.0 (GraphPad Software, San Diego, Calif.). The data were initially analyzed using one-way ANOVA and Bonferroni's post-test to compare the means of treatment groups. Differences of p<0.05 were considered statistically significant. Student's t-test was used when comparing two groups.

### REFERENCES

Anantharam V, Kitazawa M, Wagner J, Kaul S, Kanthasamy A G (2002) Caspase-3-dependent proteolytic cleavage of protein kinase Cdelta is essential for oxidative stress-mediated dopaminergic cell death after exposure to methylcyclopentadienyl manganese tricarbonyl. The Journal of neuroscience: the official journal of the Society for Neuroscience 22:1738-1751.
Barcia C, Ros C M, Annese V, Carrillo-de Sauvage M A, Ros-Bernal F, Gomez A, Yuste J E, Campuzano C M, de Pablos V, Fernandez-Villalba E, Herrero M T (2012) ROCK/Cdc42-mediated microglial motility and gliapse formation lead to phagocytosis of degenerating dopaminergic neurons in vivo. Sci Rep 2:809.
Barcia C, Ros C M, Ros-Bernal F, Gomez A, Annese V, Carrillo-de Sauvage M A, Yuste J E, Campuzano C M, de Pablos V, Fernandez-Villalba E, Herrero M T (2013) Persistent phagocytic characteristics of microglia in the substantia nigra of long-term Parkinsonian macaques. Journal of neuroimmunology 261:60-66.
Block M L, Zecca L, Hong J S (2007) Microglia-mediated neurotoxicity: uncovering the molecular mechanisms. Nat Rev Neurosci 8:57-69.
Bohnen N I, Frey K A, Studenski S, Kotagal V, Koeppe R A, Constantine G M, Scott P J, Albin R L, Muller M L (2014) Extra-nigral pathological conditions are common in Parkinson's disease with freezing of gait: An in vivo positron emission tomography study. Movement disorders: official journal of the Movement Disorder Society 29:1118-1124.
Burguillos M A, Deierborg T, Kavanagh E, Persson A, Hajji N, Garcia-Quintanilla A, Cano J, Brundin P, Englund E, Venero J L, Joseph B (2011) Caspase signalling controls microglia activation and neurotoxicity. Nature 472:319-324.
Carson M J (2002) Microglia as liaisons between the immune and central nervous systems: functional implications for multiple sclerosis. Glia 40:218-231.
Chaudhuri K R, Healy D G, Schapira A H (2006) Non-motor symptoms of Parkinson's disease: diagnosis and management. Lancet Neurol 5:235-245.
Choi H B, Ryu J K, Kim S U, McLarnon J G (2007) Modulation of the purinergic P2X7 receptor attenuates lipopolysaccharide-mediated microglial activation and neuronal damage in inflamed brain. The Journal of neuroscience: the official journal of the Society for Neuroscience 27:4957-4968.
Crotti A, Benner C, Kerman B E, Gosselin D, Lagier-Tourenne C, Zuccato C, Cattaneo E, Gage F H, Cleveland D W, Glass C K (2014) Mutant Huntingtin promotes autonomous microglia activation via myeloid lineage-determining factors. Nature neuroscience 17:513-521.
Dey N, Crosswell H E, De P, Parsons R, Peng Q, Su J D, Durden D L (2008) The protein phosphatase activity of PTEN regulates SRC family kinases and controls glioma migration. Cancer research 68:1862-1871.
Dunah A W, Sirianni A C, Fienberg A A, Bastia E, Schwarzschild M A, Standaert D G (2004) Dopamine D1-dependent trafficking of striatal N-methyl-D-aspartate glutamate receptors requires Fyn protein tyrosine kinase but not DARPP-32. Mol Pharmacol 65:121-129.
El Benna J, Faust R P, Johnson J L, Babior B M (1996) Phosphorylation of the respiratory burst oxidase subunit p47phox as determined by two-dimensional phosphopeptide mapping. Phosphorylation by protein kinase C, protein kinase A, and a mitogen-activated protein kinase. The Journal of biological chemistry 271:6374-6378.
Engelmann S, Togni M, Thielitz A, Reichardt P, Kliche S, Reinhold D, Schraven B, Reinhold A (2013) T cell-independent modulation of experimental autoimmune encephalomyelitis in ADAP-deficient mice. Journal of immunology 191:4950-4959.
Finberg R W, Yim C, Yan J, Cao L C, Mandell L, Kurt-Jones E A (2012) Phosphorylated Toll-Like Receptor 2 Interacts with Fyn and Cross-Talks with the Phosphorylation-Independent TLR2-Signaling Pathway. The Open Immunology Journal 5:36-45.
Gao H M, Zhou H, Zhang F, Wilson B C, Kam W, Hong J S (2011) HMGB1 acts on microglia Mac1 to mediate chronic neuroinflammation that drives progressive neurodegeneration. The Journal of neuroscience: the official journal of the Society for Neuroscience 31:1081-1092.
Ghosh A, Chandran K, Kalivendi S V, Joseph J, Antholine W E, Hillard C J, Kanthasamy A, Kalyanaraman B (2010) Neuroprotection by a mitochondria-targeted drug in a Parkinson's disease model. Free Radic Biol Med 49:1674-1684.
Ghosh A, Roy A, Liu X, Kordower J H, Mufson E J, Hartley D M, Ghosh S, Mosley R L, Gendelman H E, Pahan K (2007) Selective inhibition of N F-kappaB activation prevents dopaminergic neuronal loss in a mouse model of Parkinson's disease. Proceedings of the National Academy of Sciences of the United States of America 104:18754-18759.
Ghosh A, Saminathan H, Kanthasamy A, Anantharam V, Jin H, Sondarva G, Harischandra D S, Qian Z, Rana A, Kanthasamy A G (2013) The peptidyl-prolyl isomerase Pin1 up-regulation and proapoptotic function in dopaminergic neurons: relevance to the pathogenesis of Parkinson disease. The Journal of biological chemistry 288:21955-21971.
Glass C K, Saijo K, Winner B, Marchetto M C, Gage F H (2010) Mechanisms underlying inflammation in neurodegeneration. Cell 140:918-934.
Gomez G, Gonzalez-Espinosa C, Odom S, Baez G, Cid M E, Ryan J J, Rivera J (2005a) Impaired FceRI-Dependent Gene Expression and Defective Eicosanoid and Cytokine Production as a Consequence of Fyn Deficiency in Mast Cells. J Immunol 175.
Gomez G, Gonzalez-Espinosa C, Odom S, Baez G, Cid M E, Ryan J J, Rivera J (2005b) Impaired FcepsilonRI-dependent gene expression and defective eicosanoid and cytokine production as a consequence of Fyn deficiency in mast cells. Journal of immunology 175:7602-7610.
Gong P, Angelini D J, Yang S, Xia G, Cross A S, Mann D, Bannerman D D, Vogel S N, Goldblum S E (2008) TLR4 signaling is coupled to SRC family kinase activation, tyrosine phosphorylation of zonula adherens proteins, and opening of the paracellular pathway in human lung microvascular endothelia. The Journal of biological chemistry 283:13437-13449.
Gordon R, Anantharam V, Kanthasamy A G, Kanthasamy A (2012) Proteolytic activation of proapoptotic kinase protein kinase Cdelta by tumor necrosis factor alpha death receptor signaling in dopaminergic neurons during neuroinflammation. Journal of neuroinflammation 9:82.
Gordon R, Hogan C E, Neal M L, Anantharam V, Kanthasamy A G, Kanthasamy A (2011) A simple magnetic separation method for high-yield isolation of pure primary microglia. Journal of neuroscience methods 194:287-296.
Harischandra D S, Kondru N, Martin D P, Kanthasamy A, Jin H, Anantharam V, Kanthasamy A G (2014) Role of proteolytic activation of protein kinase Cdelta in the pathogenesis of prion disease. Prion 8:143-153.
Hayden M S, Ghosh S (2004) Signaling to NF-kappaB. Genes Dev 18:2195-2224.
Haynes S E, Hollopeter G, Yang G, Kurpius D, Dailey M E, Gan W B, Julius D (2006) The P2Y12 receptor regulates microglial activation by extracellular nucleotides. Nature neuroscience 9:1512-1519.
Henn A, Lund S, Hedtjarn M, Schrattenholz A, Porzgen P, Leist M (2009) The suitability of BV2 cells as alternative model system for primary microglia cultures or for animal experiments examining brain inflammation. ALTEX 26:83-94.
Hickman S E, Kingery N D, Ohsumi T K, Borowsky M L, Wang L C, Means T K, El Khoury J (2013) The microglial sensome revealed by direct RNA sequencing. Nature neuroscience 16:1896-1905.
Hirsch E C, Hunot S (2009) Neuroinflammation in Parkinson's disease: a target for neuroprotection? The Lancet Neurology 8:382-397.
Hu X, Zhang D, Pang H, Caudle W M, Li Y, Gao H, Liu Y, Qian L, Wilson B, Di Monte D A, Ali S F, Zhang J, Block M L, Hong J S (2008) Macrophage antigen complex-1 mediates reactive microgliosis and progressive dopaminergic neurodegeneration in the MPTP model of Parkinson's disease. J Immunol 181:7194-7204.
Imamura K, Hishikawa N, Sawada M, Nagatsu T, Yoshida M, Hashizume Y (2003) Distribution of major histocompatibility complex class II-positive microglia and cytokine profile of Parkinson's disease brains. Acta neuropathologica 106:518-526.
Jenner P, Olanow C W (2006) The pathogenesis of cell death in Parkinson's disease. Neurology 66:S24-36.
Jin H, Kanthasamy A, Anantharam V, Rana A, Kanthasamy A G (2011a) Transcriptional regulation of pro-apoptotic protein kinase Cdelta: implications for oxidative stress-induced neuronal cell death. The Journal of biological chemistry 286:19840-19859.
Jin H, Kanthasamy A, Ghosh A, Yang Y, Anantharam V, Kanthasamy A G (2011b) alpha-Synuclein negatively regulates protein kinase Cdelta expression to suppress apoptosis in dopaminergic neurons by reducing p300 histone acetyltransferase activity. The Journal of neuroscience: the official journal of the Society for Neuroscience 31:2035-2051.
Jin H, Kanthasamy A, Harischandra D S, Kondru N, Ghosh A, Panicker N, Anantharam V, Rana A, Kanthasamy A G (2014) Histone Hyperacetylation Up-regulates Protein Kinase Cdelta in Dopaminergic Neurons to Induce Cell Death: RELEVANCE TO EPIGENETIC MECHANISMS OF NEURODEGENERATION IN PARKINSON DISEASE. The Journal of biological chemistry 289:34743-34767.
Kaspar J W, Jaiswal A K (2011) Tyrosine phosphorylation controls nuclear export of Fyn, allowing Nrf2 activation of cytoprotective gene expression. FASEB journal: official publication of the Federation of American Societies for Experimental Biology 25:1076-1087.
Kaul S, Kanthasamy A, Kitazawa M, Anantharam V, Kanthasamy A G (2003) Caspase-3 dependent proteolytic activation of protein kinase Cdelta mediates and regulates 1-methyl-4-phenylpyridinium (MPP+)-induced apoptotic cell death in dopaminergic cells: relevance to oxidative stress in dopaminergic degeneration. European Journal of Neuroscience 18:1387-1401.
Kaul S, Anantharam V, Yang Y, Choi C J, Kanthasamy A, Kanthasamy A G (2005) Tyrosine phosphorylation regulates the proteolytic activation of protein kinase Cdelta in dopaminergic neuronal cells. The Journal of biological chemistry 280:28721-28730.
Kim C, Ho D H, Suk J E, You S, Michael S, Kang J, Joong Lee S, Masliah E, Hwang D, Lee H J, Lee S J (2013a) Neuron-released oligomeric alpha-synuclein is an endogenous agonist of TLR2 for paracrine activation of microglia. Nature communications 4:1562. Kim J H, Choi D J, Jeong H K, Kim J, Kim D W, Choi S Y, Park S M, Suh Y H, Jou I, Joe E H (2013b) DJ-1 facilitates the interaction between STAT1 and its phosphatase, SHP-1, in brain microglia and astrocytes: A novel anti-inflammatory function of DJ-1. Neurobiology of disease 60:1-10.
Kim Y S, Choi D H, Block M L, Lorenzl S, Yang L, Kim Y J, Sugama S, Cho B P, Hwang O, Browne S E, Kim S Y, Hong J S, Beal M F, Joh T H (2007) A pivotal role of matrix metalloproteinase-3 activity in dopaminergic neuronal degeneration via microglial activation. FASEB J 21:179-187.
Koistinaho M, Koistinaho J (2002) Role of p38 and p44/42 mitogen-activated protein kinases in microglia. Glia 40:175-183.
Kouadir M, Yang L, Tan R, Shi F, Lu Y, Zhang S, Yin X, Zhou X, Zhao D (2012) CD36 Participates in PrP106-126-Induced Activation of Microglia. PLOS One 7.
Lambert M P, Barlow A K, Chromy B A, Edwards C, Freed R, Liosatos M, Morgan T E, Rozovsky I, Trommer B, Viola K L, Wals P, Zhang C, Finch C E, Krafft G A, Klein W L (1998) Diffusible, nonfibrillar ligands derived from Ab1-42 are potent central nervous system neurotoxins. Proc Natl Acad Sci USA 95.
Larson M, Sherman M A, Amar F, Nuvolone M, Schneider J A, Bennett D A, Aguzzi A, Lesne S E The complex PrP(c)-Fyn couples human oligomeric Abeta with pathological tau changes in Alzheimer's disease. The Journal of neuroscience: the official journal of the Society for Neuroscience 32:16857-16871a.
Lastres-Becker I, Ulusoy A, Innamorato N G, Sahin G, Rabano A, Kirik D, Cuadrado A (2012) alpha-Synuclein expression and Nrf2 deficiency cooperate to aggravate protein aggregation, neuronal death and inflammation in early-stage Parkinson's disease. Human molecular genetics 21:3173-3192.
Latchoumycandane C, Anantharam V, Jin H, Kanthasamy A, Kanthasamy A (2011) Dopaminergic neurotoxicant 6-OHDA induces oxidative damage through proteolytic activation of PKCdelta in cell culture and animal models of Parkinson's disease. Toxicology and applied pharmacology 256:314-323.
Lee S, Nam Y, Koo J Y, Lim D, Park J, Ock J, Kim J, Suk K, Park S B (2014) A small molecule binding HMGB1 and HMGB2 inhibits microglia-mediated neuroinflammation. Nature chemical biology 10:1055-1060.
Levy O A, Malagelada C, Greene L A (2009) Cell death pathways in Parkinson's disease: proximal triggers, distal effectors, and final steps. Apoptosis 14:478-500.
Moore K J, El Khoury J, Medeiros L A, Terada K, Geula C, Luster A D, Freeman M W (2002) A CD36-initiated signaling cascade mediates inflammatory effects of beta-amyloid. The Journal of biological chemistry 277:47373-47379.
Olanow C W (2007) The pathogenesis of cell death in Parkinson's disease—2007. Mov Disord 22 Suppl 17:S335-342.
Olson J K, Miller S D (2004) Microglia Initiate Central Nervous System Innate and Adaptive Immune Responses through Multiple TLRs. J Immunol 173:3916-3924.
Palacios E H, Weiss A (2004) Function of the Src-family kinases, Lck and Fyn, in T-cell development and activation. Oncogene 23:7990-8000.
Parameswaran N, Patial S (2010) Tumor Necrosis Factor-a Signaling in Macrophages. Crit Rev Eukaryot Gene Expr 20:87-103.
Przedborski S (2005) Pathogenesis of nigral cell death in Parkinson's disease. Parkinsonism & related disorders 11 Suppl 1:S3-7.
Przedborski S, Tieu K, Perier C, Vila M (2004) MPTP as a mitochondrial neurotoxic model of Parkinson's disease. J Bioenerg Biomembr 36:375-379.
Puschmann A (2013) Monogenic Parkinson's disease and parkinsonism: clinical phenotypes and frequencies of known mutations. Parkinsonism & related disorders 19:407-415.
Qin L, Wu X, Block M L, Liu Y, Breese G R, Hong J S, Knapp D J, Crews F T (2007) Systemic LPS causes chronic neuroinflammation and progressive neurodegeneration. Glia 55:453-462.
Raj asekaran K, Kumar P, Schuldt K M, Peterson E J, Vanhaesebroeck B, Dixit V, Thakar M S, Malarkannan S (2013) Signaling by Fyn-ADAP via the Carmal-Bcl-10-MAP3K7 signalosome exclusively regulates inflammatory cytokine production in N K cells. Nature immunology 14:1127-1136.
Reed-Geaghan E G, Savage J C, Hise A G, Landreth G E (2009) CD14 and toll-like receptors 2 and 4 are required for fibrillar A{beta}-stimulated microglial activation. The Journal of neuroscience: the official journal of the Society for Neuroscience 29:11982-11992.
Saminathan H, Asaithambi A, Anantharam V, Kanthasamy A G, Kanthasamy A (2011) Environmental neurotoxic pesticide dieldrin activates a non receptor tyrosine kinase to promote PKCdelta-mediated dopaminergic apoptosis in a dopaminergic neuronal cell model. Neurotoxicology 32:567-577.
Sperber B R, Boyle-Walsh E A, Engleka M J, Gadue P, Peterson A C, Stein P L, Scherer S S, McMorris F A (2001) A Unique Role for Fyn in CNS Myelination. The Journal of neuroscience: the official journal of the Society for Neuroscience 21:2039-2047.
Sriram K, Matheson J M, Benkovic S A, Miller D B, Luster M I, O'Callaghan J P (2006) Deficiency of TNF receptors suppresses microglial activation and alters the susceptibility of brain regions to MPTP-induced neurotoxicity: role of TNF-alpha. FASEB journal: official publication of the Federation of American Societies for Experimental Biology 20:670-682.
Steinberg S F (2004) Distinctive activation mechanisms and functions for protein kinase Cδ. Biochem J 384:449-459.
Stott S R, Barker R A (2014) Time course of dopamine neuron loss and glial response in the 6-OHDA striatal mouse model of Parkinson's disease. Eur J Neurosci 39:1042-1056.
Stuart L M, Bell S A, Stewart C R, Silver J M, Richard J, Goss J L, Tseng A A, Zhang A, El Khoury J B, Moore K J (2007) CD36 signals to the actin cytoskeleton and regulates microglial migration via a p130Cas complex. The Journal of biological chemistry 282:27392-27401.
Su X, Maguire-Zeiss K A, Giuliano R, Prifti L, Venkatesh K, Federoff H J (2008) Synuclein activates microglia in a model of Parkinson's disease. Neurobiology of aging 29:1690-1701.
Tansey M G, Goldberg M S (2010) Neuroinflammation in Parkinson's disease: its role in neuronal death and implications for therapeutic intervention. Neurobiology of disease 37:510-518.
Um J W, Nygaard H B, Heiss J K, Kostylev M A, Stagi M, Vortmeyer A, Wisniewski T, Gunther E C, Strittmatter S M Alzheimer amyloid-beta oligomer bound to postsynaptic prion protein activates Fyn to impair neurons. Nature neuroscience 15:1227-1235.
Virgone-Carlotta A, Uhlrich J, Akram M N, Ressnikoff D, Chretien F, Domenget C, Gherardi R, Despars G, Jurdic P, Honnorat J, Nataf S, Touret M (2013) Mapping and kinetics of microglia/neuron cell-to-cell contacts in the 6-OHDA murine model of Parkinson's disease. Glia 61:1645-1658.
Wake H, Lee P R, Fields R D (2011) Control of local protein synthesis and initial events in myelination by action potentials. Science 333:1647-1651.
Wu D C, Teismann P, Tieu K, Vila M, Jackson-Lewis V, Ischiropoulos H, Przedborski S (2003) NADPH oxidase mediates oxidative stress in the 1-methyl-4-phenyl-1,2,3,6-tetrahydropyridine model of Parkinson's disease. Proceedings of the National Academy of Sciences of the United States of America 100:6145-6150.
Wu D C, Jackson-Lewis V, Vila M, Tieu K, Teismann P, Vadseth C, Choi D K, Ischiropoulos H, Przedborski S (2002) Blockade of microglial activation is neuroprotective in the 1-methyl-4-phenyl-1,2,3,6-tetrahydropyridine mouse model of Parkinson disease. The Journal of neuroscience: the official journal of the Society for Neuroscience 22:1763-1771.
Zhang D, Anantharam V, Kanthasamy A, Kanthasamy A G (2007) Neuroprotective effect of protein kinase C delta inhibitor rottlerin in cell culture and animal models of Parkinson's disease. The Journal of pharmacology and experimental therapeutics 322:913-922.

### Example 2

Neuroinflammation has been implicated as a major pathophysiological process of Parkinson's disease (PD) in recent years. Among various neuroinflammatory triggers, protein aggregates have been shown to be a predominant pathological trigger for microglial activation and subsequent proinflammatory cytokine and chemokine production in the brain, which in turn contributes to the accelerated progression of neurodegenerative processes. Also, emerging evidence indicates that aggregated pathogenic proteins, including α-synuclein (αSyn), are packaged into exosomes, which propagate protein aggregates from affected neurons to other brain cells, including microglial cells, through a non-cellular autonomous process, leading to a heightened neuroinflammatory response. Despite these advances, the cellular mechanisms underlying microglia-mediated neuroinflammatory events following stimulation with αSyn aggregates and αSyn-containing exosomes are yet to be defined.

Although endogenous αSyn protein is involved in many synaptic functions, misfolded αSyn serves as an endogenous antigen, provoking a self-propelling cycle of glial activity and upregulated proinflammatory mediators under both in vitro and in vivo conditions (1, 7-10). Emerging evidence indicates that aggregated extracellular αSyn can spread via a prion-like transmission mechanism, thereby spreading αSyn pathology to neighboring neuronal and glial cells (2, 3, 8, 11-14). Though its secretory mechanisms remain controversial, the exosomal release of pathogenic αSyn has been emerging as a novel concept in CNS diseases progression (2, 4, 5, 15). Also, αSyn aggregates are known to interact with microglial immune receptors. Recently, Kim et al. provided evidence that TLR2, but not TLR3 or TLR4, acts as a receptor for extracellular αSyn in microglia (16). Additionally, the pattern recognition receptor CD36 is involved in αSyn-mediated microglial activation (17). Thus, αSyn aggregates can activate TLR2/CD36 receptors. Nevertheless, the key signaling pathways involved in aggregated αSyn-mediated neuroinflammation in the nigral dopaminergic system are largely unknown. This idea of extracellular αSyn as an immunogenic agent to glial cells is extremely appealing given its emerging role in disease progression. Hence, the novel goal of this study is to characterize the role of a major non-receptor tyrosine kinase, Fyn, in regulating αSyn aggregate-induced neuroinflammation in PD. Apart from providing novel insights into the signaling mechanisms mediating neuroinflammatory responses during protein aggregation in PD pathology, this study also provides translational potential for developing neuroprotective strategies targeting Fyn kinase signaling during neuroinflammation in PD related neurodegenerative diseases.

### Results

Aggregated Human α-Synuclein Acts as an Efficient Danger Signal for the NLRP3 Inflammasome, Inducing IL-1β Maturation and Release

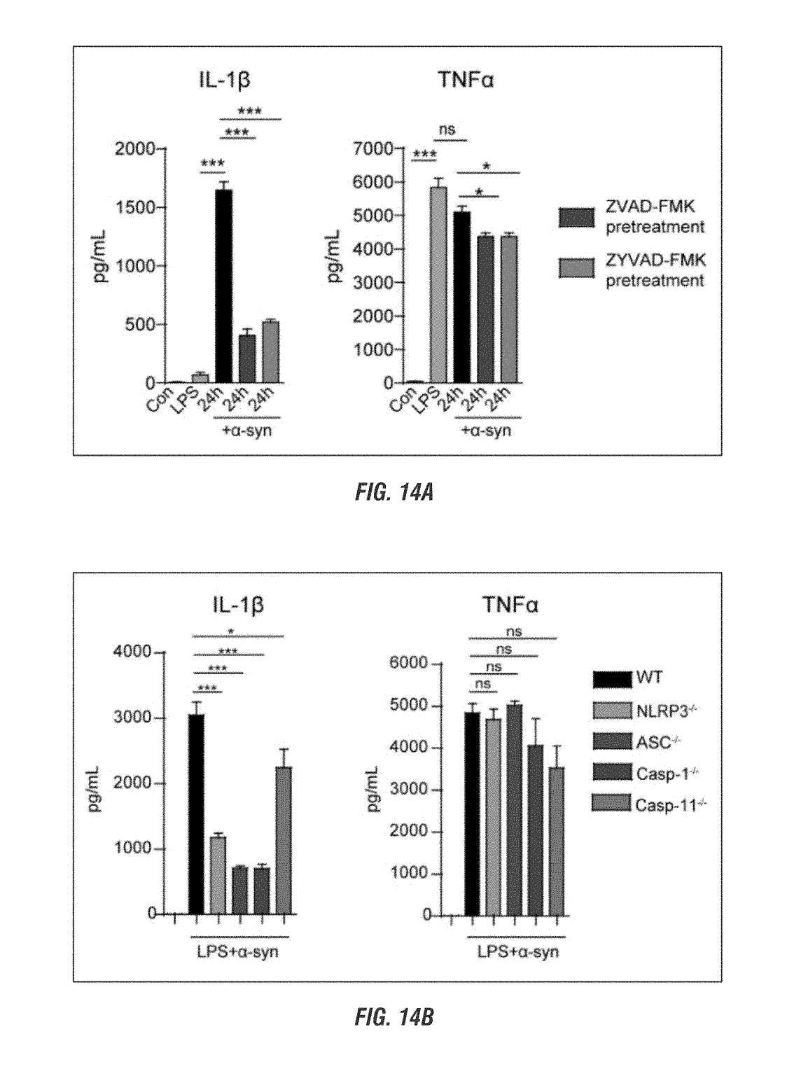

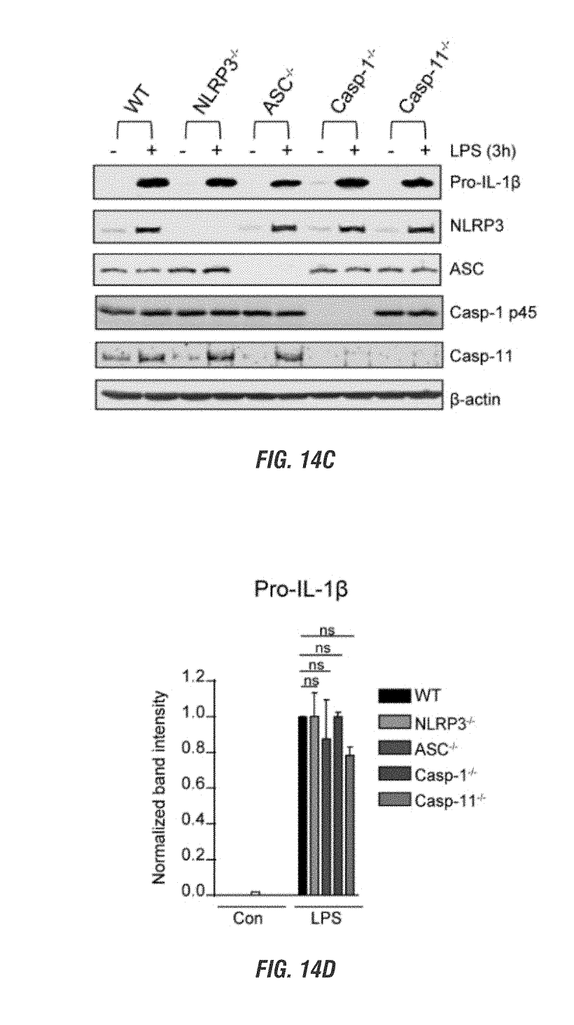

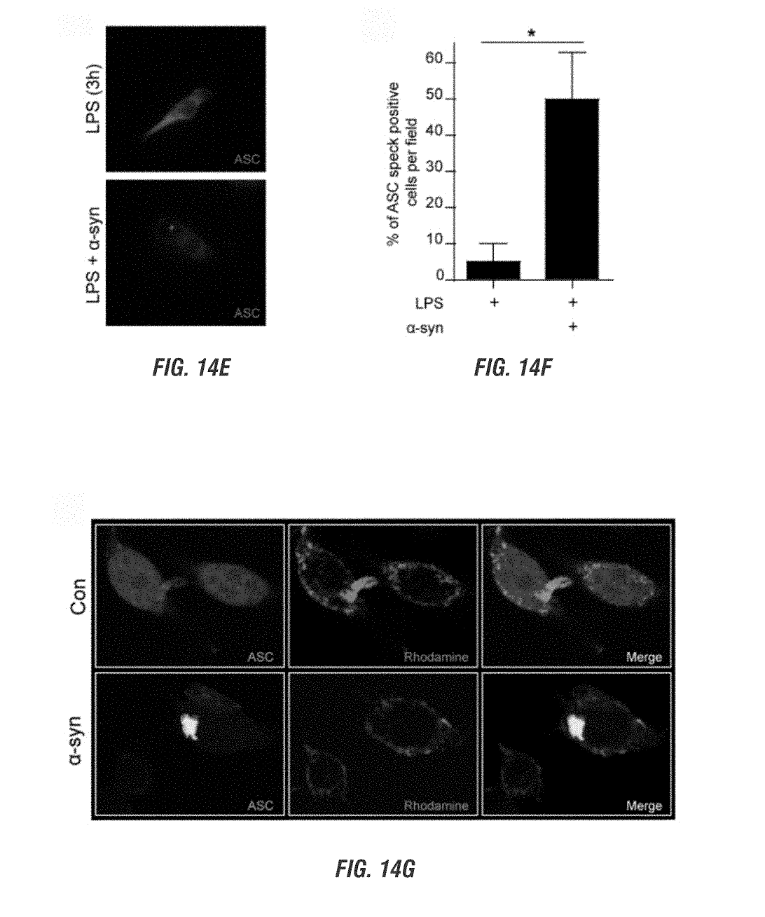

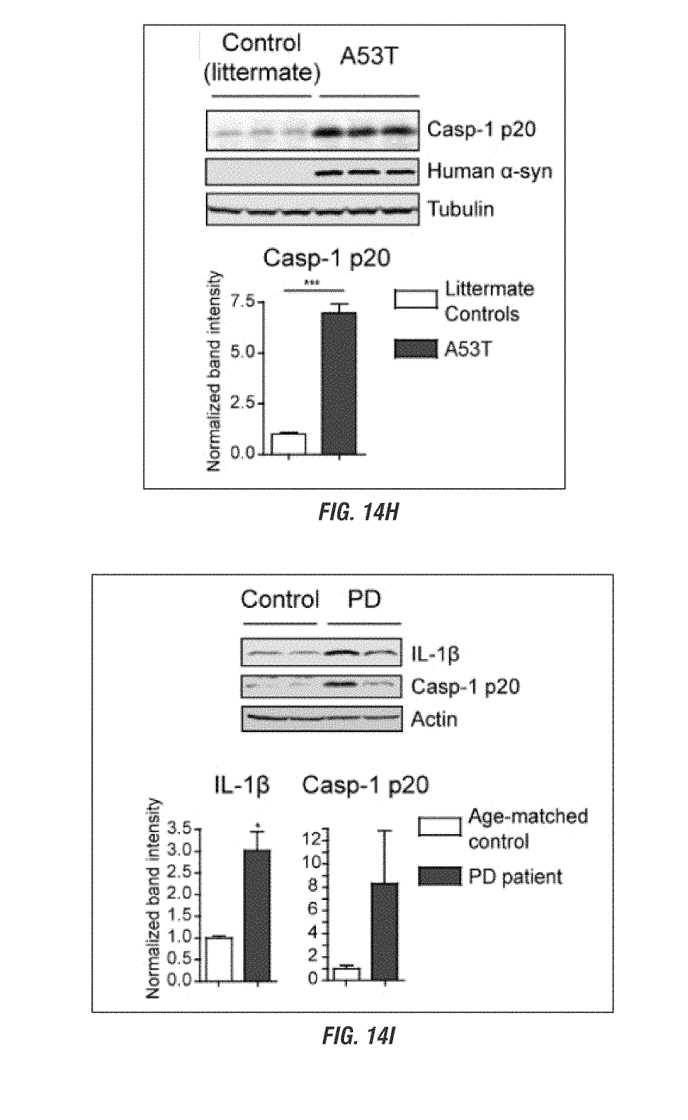

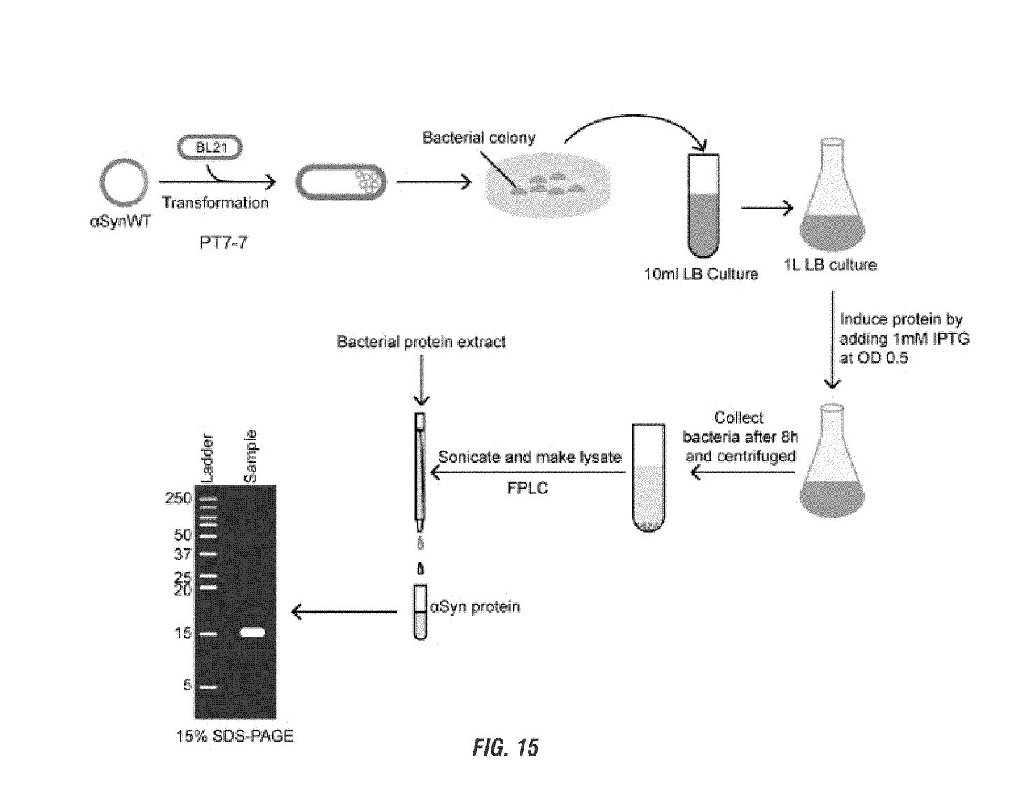

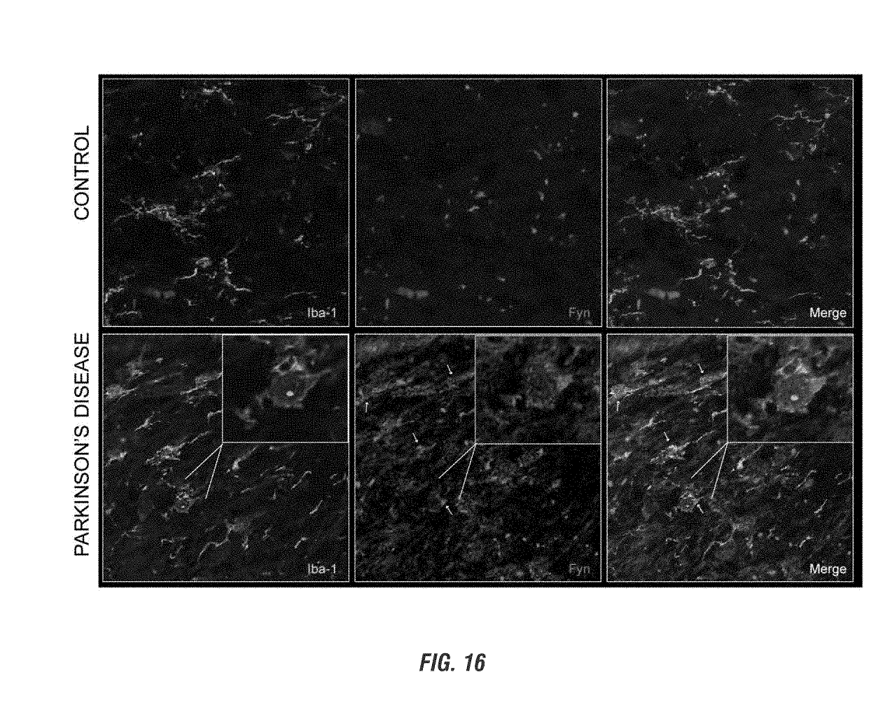

Misfolded proteins can activate the NLRP3 inflammasomes in microglia (Hafner-Bratkovic et al., 2012; Halle et al., 2008). After obtaining and purifying human αSyn (FIG. 15), we determined that endotoxin contamination was minimal, and that an equivalent concentration of LPS could not activate pro-inflammatory signaling in microglia (data not shown). Aggregation of αSyn was performed as previously described (Zhang et al., 2005). LPS-primed primary murine microglia were treated with α-synuclein for 24 h, with or without the pre-treatment of the pan-Caspase inhibitor ZVAD-FMK or the Caspase-1 specific inhibitor ZYVAD-FMK. Treatment with αSyn sharply induced IL-1β cleavage and secretion, which was reduced in the group pre-treated with the Caspase inhibitors (FIG. 14A). Secretion/production of TNFα in the αSyn-treated cells did not significantly change when compared to the LPS primed group and was only marginally reduced in the Caspase-1 and pan-Caspase inhibitor pre-treated groups. Next, we treated LPS-primed bone marrow-derived macrophages (BMDMs) from WT, NLRP3^{-/-}, ASC^{-/-}, Caspase-1^{-/-} and Caspase-11^{-/-} mice with aggregated human α-synuclein for 24 h (FIG. 14B). Secretion of IL-1β from NLRP3-, ASC- and Caspase-1-deficient macrophages was dramatically reduced, but not from Caspase-11-deficient macrophages (Caspase-11^{-/-} macrophages were used as a control, since the Caspase-1^{-/-} mouse line inherently lacks Caspase-11). The levels of supernatant TNFα did not change in any of the genotypes. By immunoblotting the levels of pro-IL-1β produced upon LPS treatment (FIG. 14C, 14D), we confirmed that none of these proteins played a role in the priming of the NLRP3 inflammasome. Next, we assessed the inflammasome activating ability of aggregated human αSyn in primary murine microglia by staining for ASC in LPS and LPS+αSyn treated microglia. LPS treatment alone did not elicit SC oligomerization, but the number of cells displaying ASC specks significantly increased in the LPS+αSyn treated group (FIG. 14E, 14F). We also utilized the ASC-CFP reporter cell line, which overexpresses ASC tagged with cyan fluorescent protein (CFP). NLRP3 inflammasome activation causes ASC to coalesce into a single speck within each cell. We counted the number of ASC-positive specks to measure inflammasome activation. ASC-CFP cells were primed with LPS and treated with aggregated αSyn for 2 h. Endogenous CFP fluorescence was assessed. In the αSyn treated group, specks of ASC were observed, indicating inflammasome activation (FIG. 14G). NLRP3 inflammasome activation was conclusively demonstrated in the APP/PS-1 AD model, as well as in post-mortem AD brains (Heneka et al., 2013), as indicated by increased levels of cleaved Caspase-1. Accordingly, we detected significantly increased cleaved Caspase-1 levels in the striatal lysates of 4-month-old A53T mice and their littermate controls, indicating that αSyn aggregation/overexpression can elicit inflammasome activation in vivo (FIG. 14H). Lastly, both Caspase-1 and IL-1β levels were significantly increased in the human PD nigral lysates when compared to those of control patients (FIG. 14I). In summary, aggregated αSyn acts as an efficient danger signal to activate the NLRP3 inflammasome. Data from the A53T model and from PD patient lysates provide in vivo relevance as well. We also observed increased Fyn expression within the microglia in PD tissues, when compared to age-matched control tissues (FIG. 16).

### α-Synuclein Amplifies LPS-Induced Priming of the NLRP3 Inflammasome in a Fyn-Dependent Manner

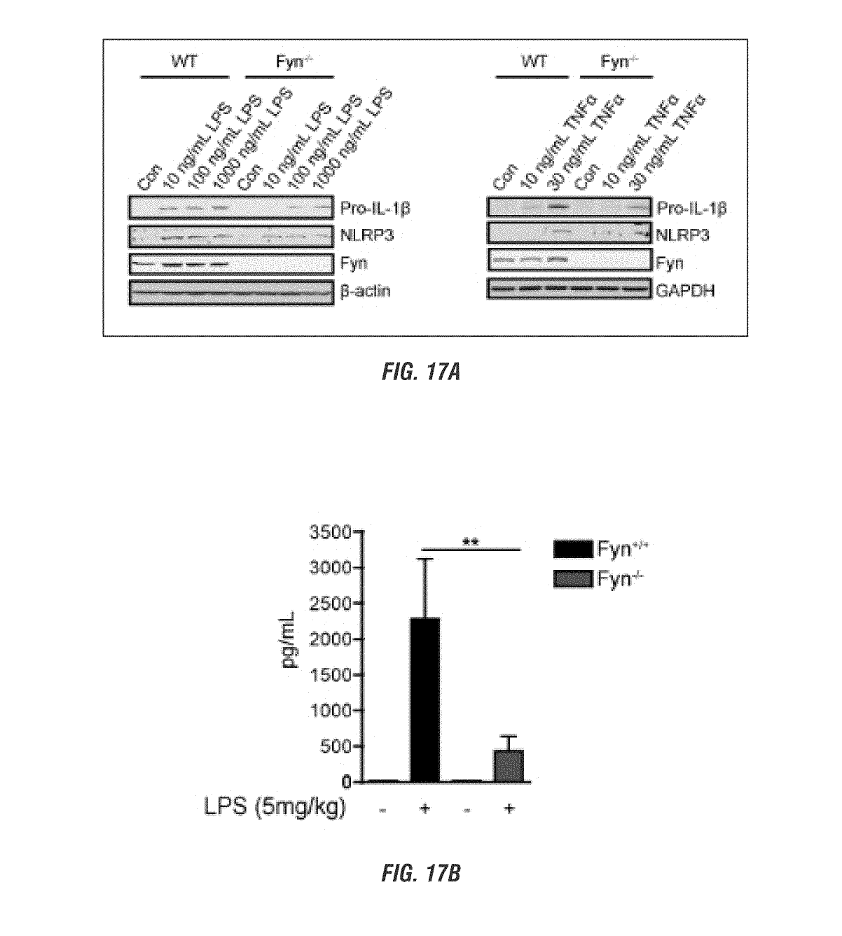

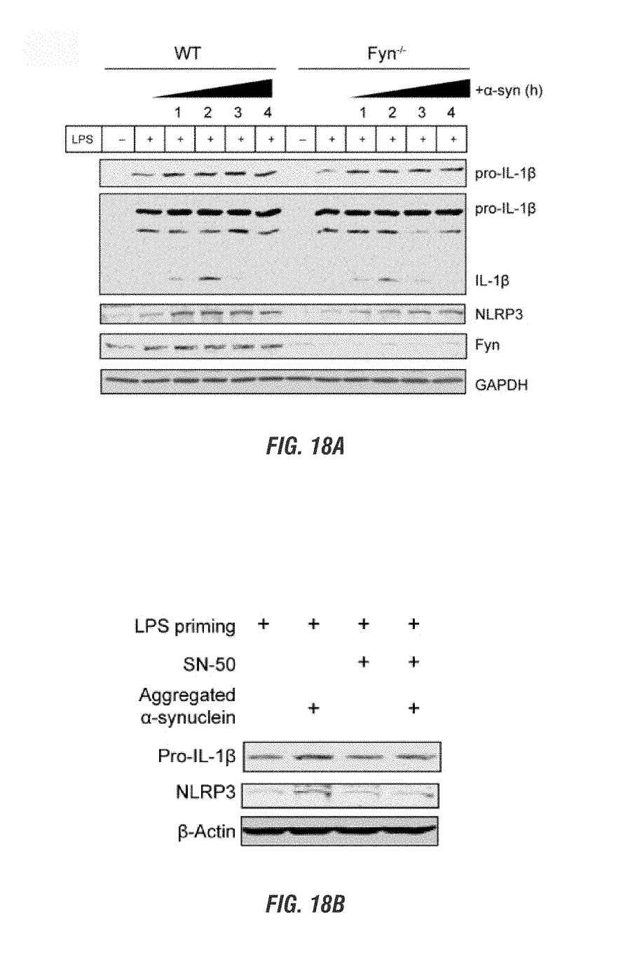

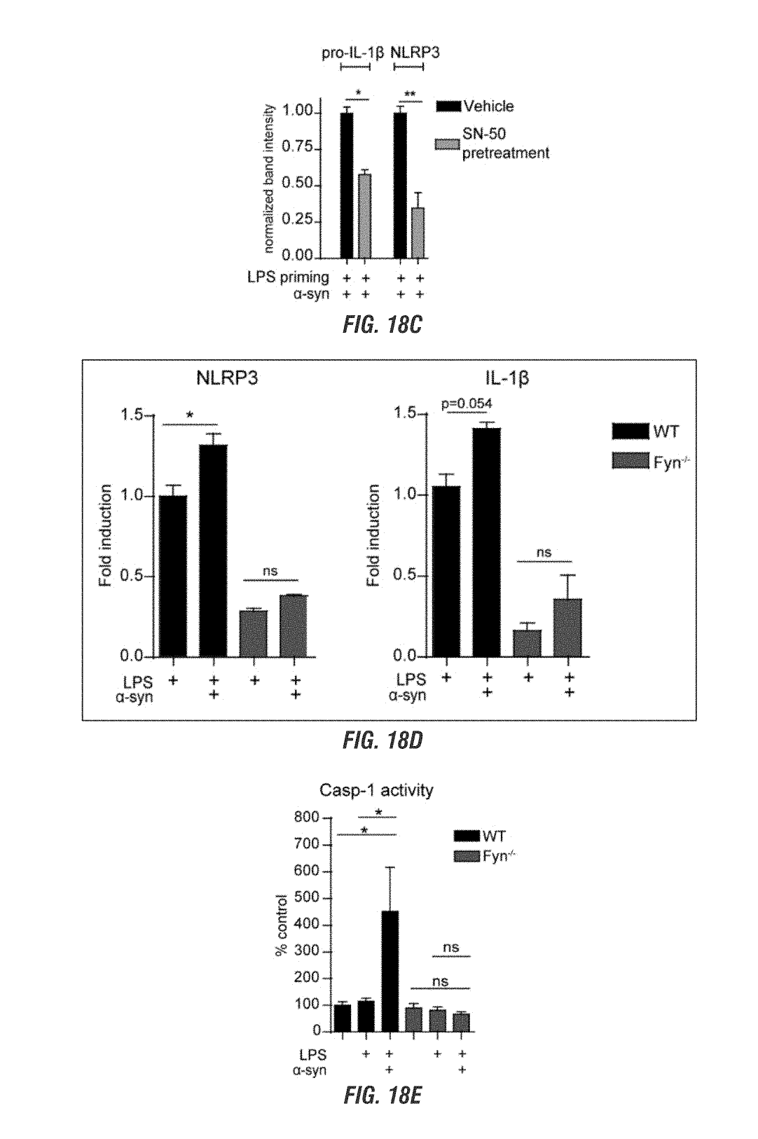

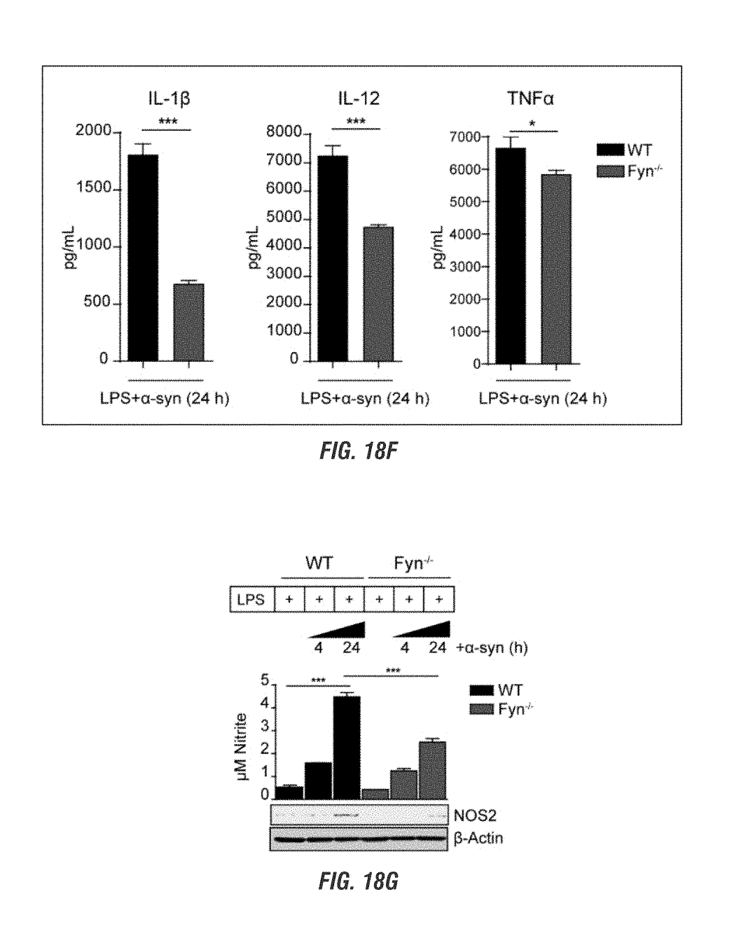

The first step in the activation process of the NLRP3 inflammasome is priming, which entails the NF-κB-p65-mediated induction of pro-IL-1β and NLRP3, which occurs subsequent to the engagement of appropriate pro-inflammatory ligands or inflammogens to their respective receptors (Hayden and Ghosh, 2004). We demonstrated in Example 1 how inflammogen stimulation of microglia resulted in rapid Fyn activation, and Fyn-mediated PKCδ tyrosine phosphorylation and activation, which contributed to LPS- and TNFα-mediated cytokine production and pro-inflammatory signaling (Panicker et al., 2015). Accordingly, we primed WT and Fyn^{-/-} microglia with various doses of LPS and TNFα to test the role of Fyn in NLRP3 inflammasome priming as assessed by blotting for pro-IL-1β and NLRP3. As expected, LPS and TNFα elicited a dose-dependent increase of pro-IL-1β and NLRP3 levels in WT microglia in contrast to a significantly lower response in Fyn^{-/-} microglia (FIG. 17A, 17B). We next treated WT and Fyn^{-/-} mice with 5 mg/kg LPS for 24 h and determined via Luminex that the LPS-mediated serum IL-1β production in the Fyn^{-/-} mice was strongly attenuated (FIG. 17B). Having established αSyn as an efficient danger signal of the inflammasome, we investigated the mechanisms through which it might activate the NLRP3 inflammasome. Immunoblot analysis of LPS-primed, αSyn-treated WT and Fyn^{-/-} microglia revealed that αSyn actually amplified the LPS induction of WT pro-IL-1β and NLRP3 levels in contrast the much more muted response in Fyn^{-/-} microglia (FIG. 18A), leading us to hypothesize that aggregated αSyn directly potentiated LPS priming by further activating the NF-κB pathway. The αSyn-mediated induction of pro-IL-1β and NLRP3 in microglia was abolished in cells pre-treated (post-LPS priming) with SN-50, an NF-κB inhibitor (FIG. 18B, 18C). Next, using LPS- and LPS/αSyn-treated WT and Fyn^{-/-} microglia, we determined that αSyn treatment induced the expression of pro-IL-1β and NLRP3 mRNAs in WT microglia, whereas levels of the respective mRNA in the Fyn^{-/-} microglia were consistently lower (FIG. 18D). The well utilized FLICA assay was then employed on primed WT and Fyn^{-/-} microglia. The αSyn-mediated induction of Caspase-1 activity was strongly induced in WT microglia, but not at all in Fyn-deficient microglia, strengthening our view that Fyn played a role in the inflammasome activation (FIG. 18E). The αSyn-mediated secretion of IL-1β and various other pro-inflammatory cytokines was also diminished in Fyn^{-/-} microglia (FIG. 18F). NOS2, the rate-limiting enzyme mediating nitrite production in various immune cells, can be induced in microglia following amyloid stimulation and in APP/PS1 mice (Heneka et al., 2013). We show that αSyn treatment of LPS-primed microglia also significantly induced NOS2 and increased supernatant nitrite levels much more in WT than in Fyn^{-/-} microglia (FIG. 18G). Taken together, our results indicate that aggregated human αSyn amplified LPS-induced priming, while simultaneously acting as a danger signal of the NLRP3 inflammasome, culminating in the release of mature IL-1β and nitrite, in a signaling pathway that utilizes Fyn kinase.

### Aggregated Human αSyn Primes and Activates the NLRP3 Inflammasome, Resulting in IL-1β Processing and Secretion

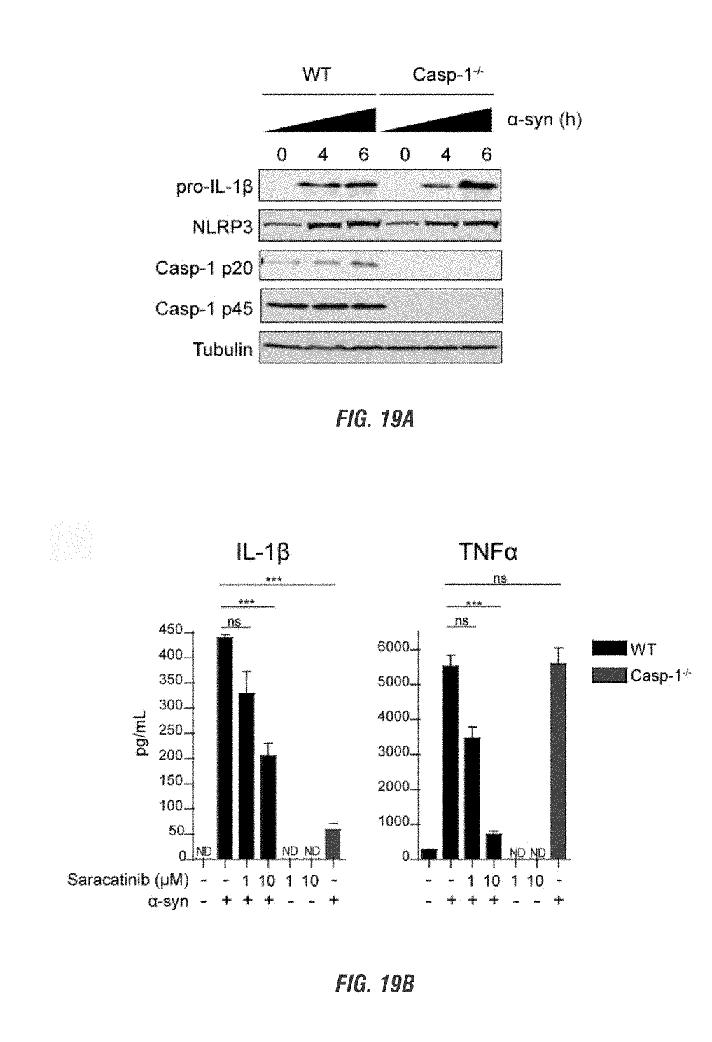

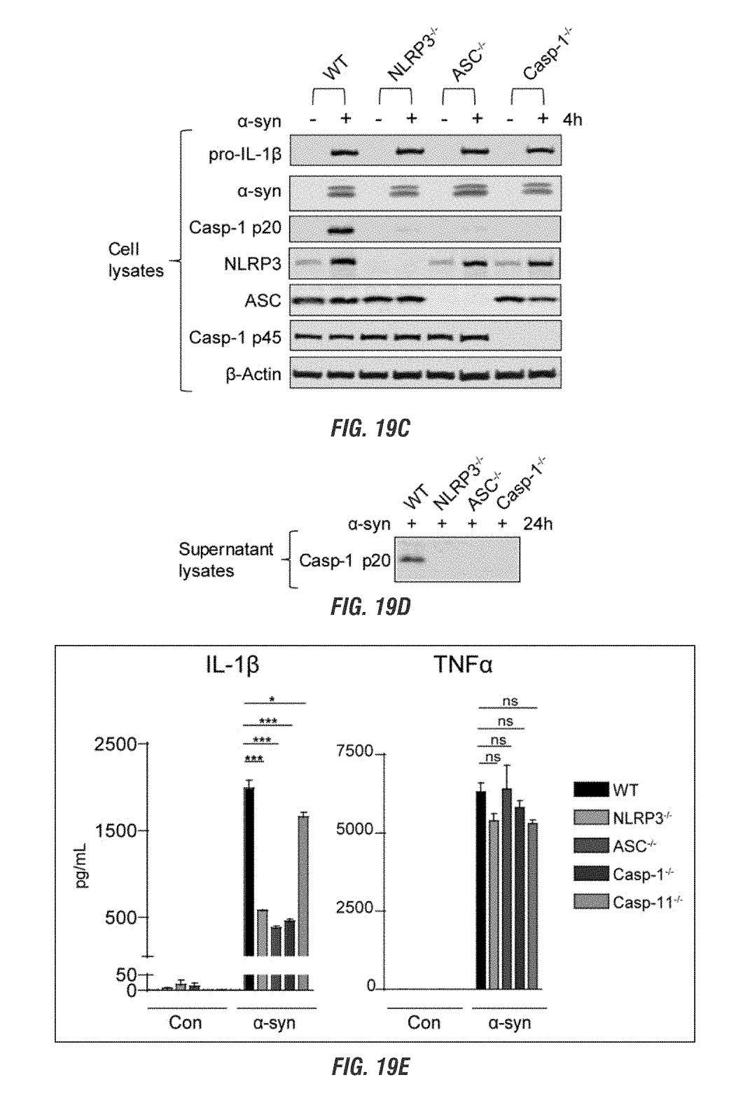

The current model of the NLRP3 inflammasome postulates a two-step mechanism. A TLR or TNFR1 agonist, acting as the initial signal, induces the expression of pro-IL-13 and NLRP3 proteins. Subsequently, a second signal causes lysosomal rupture, mitochondrial ROS generation and/or K⁺ efflux that acts as a danger signal resulting in the assembly and activation of the inflammasome complex and the processing and secretion of IL-1β into the cell supernatant. Since we observed that αSyn can signal the inflammasome and can also amplify LPS-mediated activation of NLRP3 and pro-IL-1β at the mRNA and protein level, we wondered if it could activate the NLRP3 inflammasome independent of an LPS-mediated priming step. Unprimed and immortalized WT and Capsase-1^{-/-} microglial cells were treated with aggregated αSyn for 4 h. As assessed via immunoblot, aggregated αSyn induced pro-IL-1β and NLRP3 to equivalent levels in both cell types (FIG. 19A). It also brought about the secretion of IL-1β in WT, and to a strikingly lower extent in the Caspase-1 deficient microglial cells. The secretion of IL-1β could also be inhibited by pre-treatment with the Fyn inhibitor Saracatinib in a dose-dependent manner (FIG. 19B). We next utilized primary BMDMs from WT, NLRP3^{-/-}, Caspase-1^{-/-} and Caspase-11^{-/-} mice to test the ability of αSyn to activate the NLRP3 inflammasome in them. As expected, we saw no genotypic difference in the ability of macrophages to induce pro-IL-1β and import αSyn (FIG. 19C). However, the αSyn-mediated processing of Caspase-1 was almost completely absent in ASC^{-/-}, NLRP3^{-/-} and Caspase-1^{-/-} macrophages. This was further evidenced using a Caspase-1 immunoblot from 24 hr αSyn treated WT, NLRP3^{-/-}, ASC^{-/-}, Caspase-1^{-/-} cell supernatants. Only the WT BMDM supernatant contained cleaved Caspase-1 (FIG. 19D) Supernatant analysis of unprimed macrophages treated with αSyn for 12 h showed that αSyn elicited robust IL-1β production from WT macrophages (FIG. 19E). In contrast, IL-1β production was severely diminished in NLRP3^{-/-}, ASC^{-/-} and Caspase-1^{-/-} macrophages, although it was largely restored in the Caspase-11^{-/-} macrophages. The αSyn-mediated TNFα production was not statistically different in any of the cell types.

### Aggregated αSyn Treatment Rapidly Activates Fyn-PKCδ Signaling

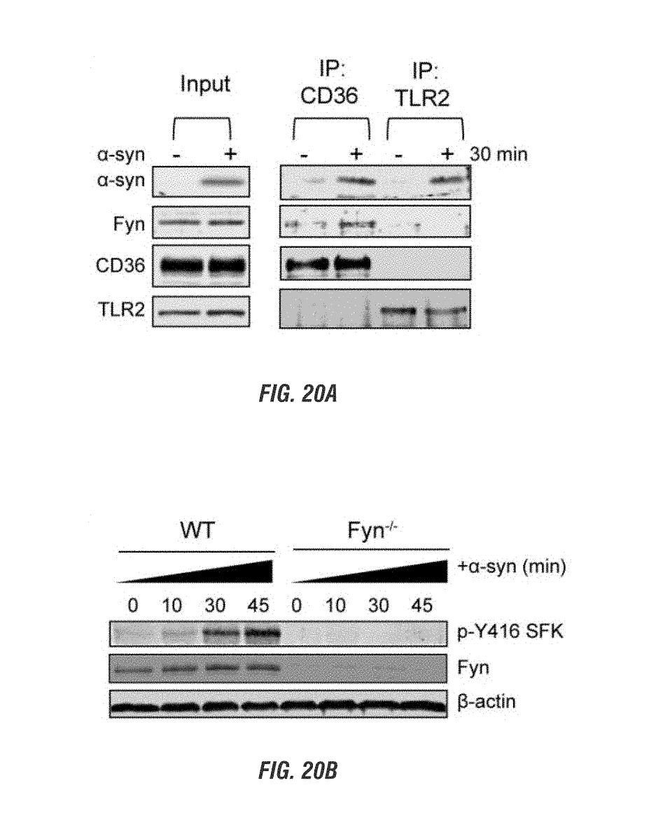

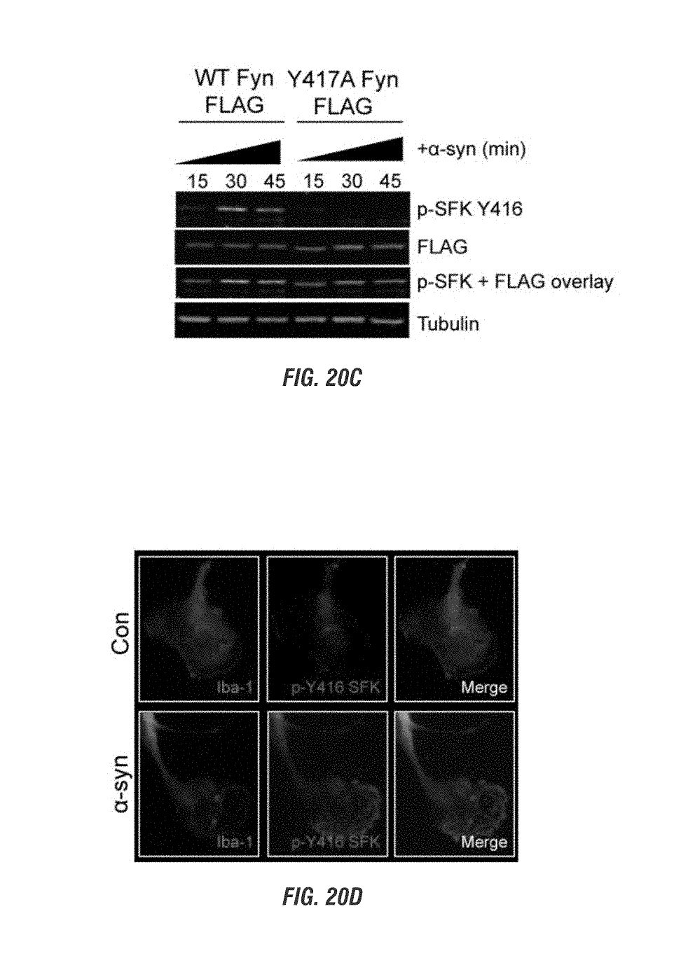

We then looked for the signaling mechanism through which αSyn activates the NLRP3 inflammasome in microglia. Various studies have indirectly implicated disparate receptors in binding to and mediating αSyn signaling. Kim et al. (2013) considered TLR2 essential for αSyn-induced pro-inflammatory signaling and to contribute to αSyn import into microglia. TLR4-, CD36- and FcγR-deficient microglia have demonstrated attenuated neuroinflammatory responses to αSyn treatment (Fellner et al., 2013). Misfolded αSyn also interacts with microglial TLR1/2 and mediates Myd-88-dependent pro-inflammatory signaling (Daniele et al., 2015). Since we had previously demonstrated early inflammogen-mediated activation of Fyn and PKCδ, we sought to link αSyn recognition by a microglial receptor to Fyn activation, which is known to occur downstream of CD36 (Chen et al., 2008; Moore et al., 2002) and TLR2 (Finberg et al., 2012). We immunoprecipitated CD36 and TLR2 in control and αSyn-stimulated WT microglia and discovered that both CD36 and TLR2 interacted with αSyn, but only CD36 interacted with Fyn in αSyn-stimulated microglia (FIG. 20A). Also, early αSyn stimulation of primary microglia rapidly induced the active loop phosphorylation of Fyn (FIG. 20B, 20C). We had used the p-Y416 Src family kinase (p-Y416 SFK) antibody to recognize activated Fyn. Since this antibody recognizes all active Src family kinases, we verified that αSyn did not induce any discernable Src kinase activation in Fyn^{-/-} microglia. We also treated primary microglial cells overexpressing FLAG-tagged WT and activation loop mutant Fyn (Y417A Fyn) with aggregated αSyn for 15, 30 and 45 min. Whole cell lysates were probed for FLAG and p-SFKY416 antibodies. WT Fyn-FLAG transfected cells demonstrated a rapid induction of p-Y416 SFK levels, which was abolished in the Y417A Fyn-FLAG transfected groups. The FLAG and p-Y416 SFK levels were assessed using secondary antibodies on the red and green channels, respectively. The FLAG and p-Y416 SFK bands perfectly co-localized in the WT-Fyn transfected cells, indicating that Fyn was the Src family kinase preferentially activated in aggregated αSyn-stimulated cells. We have previously demonstrated in microglial cells that activated Fyn associates with the serine threonine kinase PKCδ and mediates its phosphorylation via tyrosine at residue Y311 (Example 1). The αSyn-mediated p-Y311 PKCδ levels were diminished in Fyn-deficient microglia, suggesting a conserved pro-inflammatory signaling pathway downstream of inflammogen activation. Lastly, ICC analysis showed that p-Y416 SFK levels rapidly increased in microglial cells stimulated with aggregated αSyn. Active Fyn is preferentially localized along the membrane periphery, which is to be expected since it is activated rapidly following association with membrane-bound CD36.

### Fyn Contributes to Aggregated αSyn-Mediated NF-κB Activation, Contributing to Priming of the NLRP3 Inflammasome

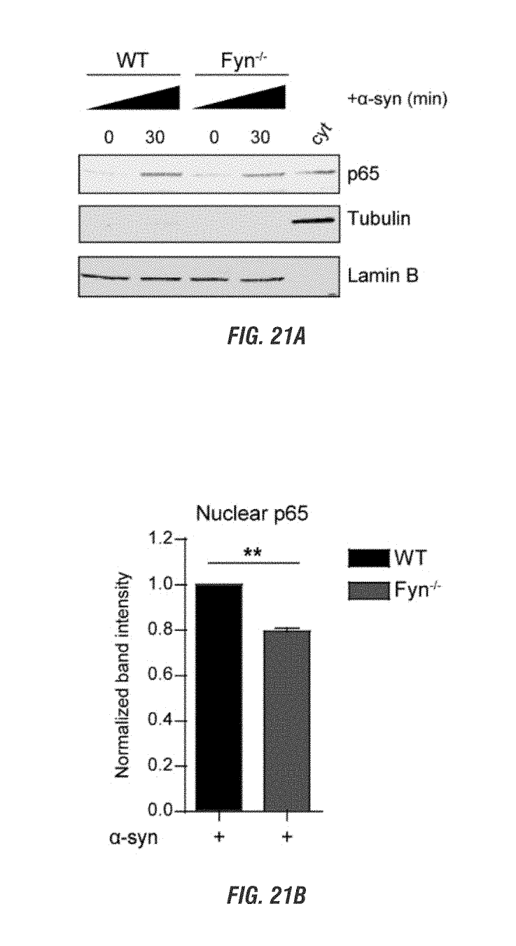

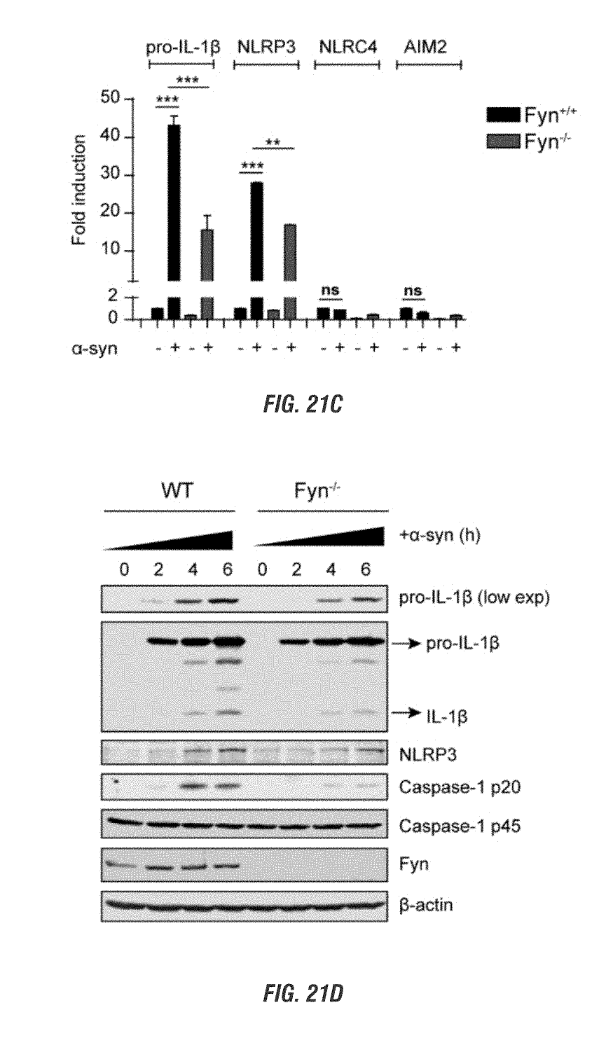

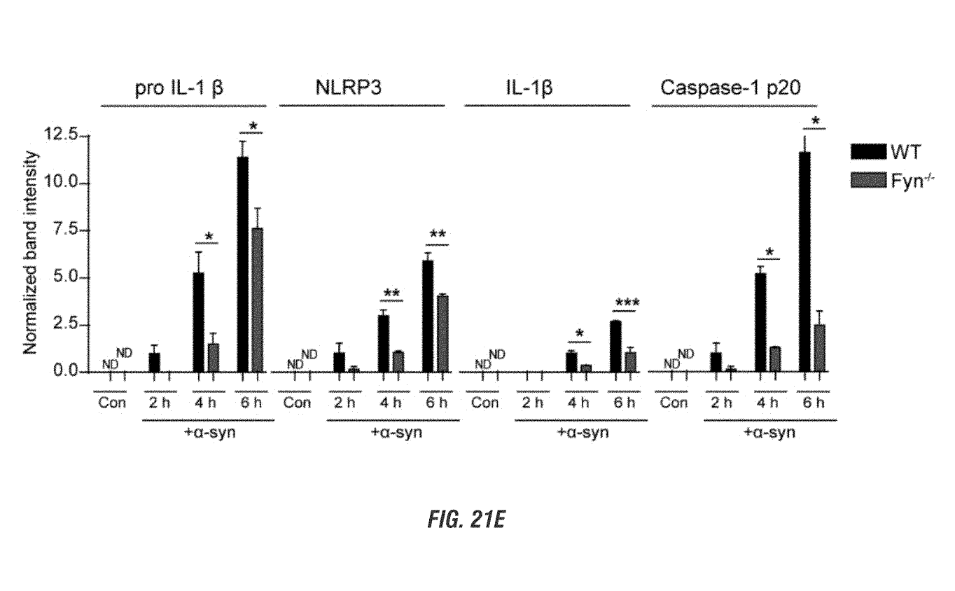

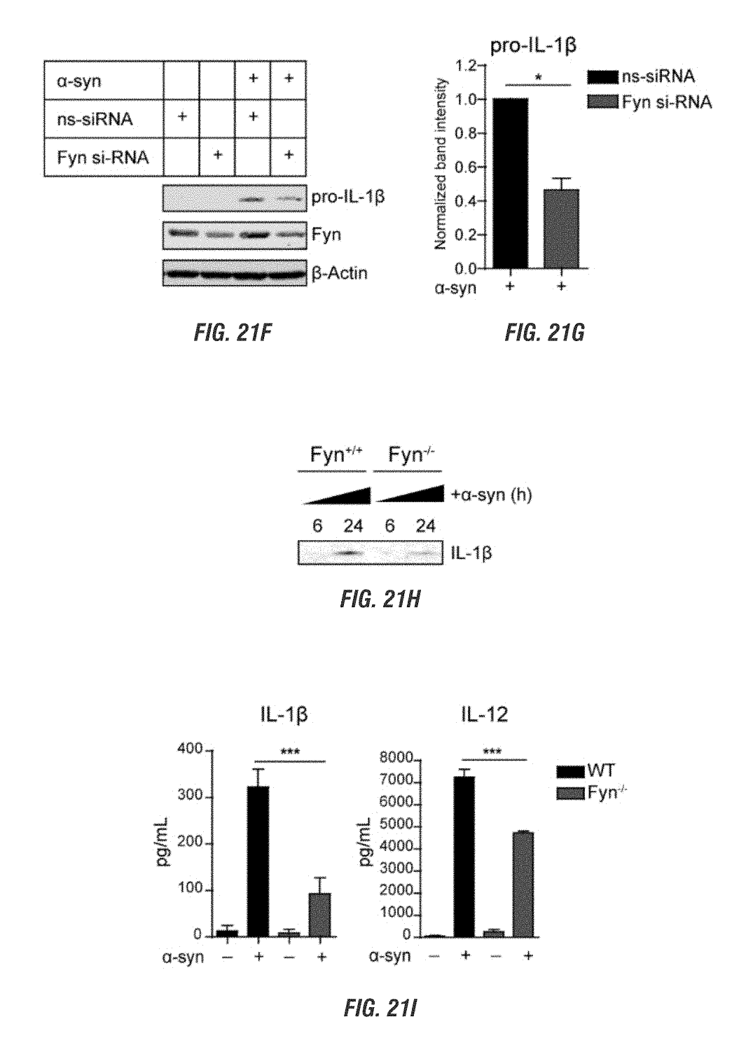

Priming of the NLRP3 inflammasome involves activation of the NF-κB pathway downstream of TLR/TNFR1 engagement of their respective ligands. The NF-κB heterotrimeric complex comprises IκBα, p65 and p50. Upon inflammogen stimulation, IκBα is phosphorylated and rapidly degraded, allowing nuclear entry of the p65-NF-κB subunit. The transcription factor p65 binds to the promoters of various pro-inflammatory cytokine genes as well as the NLRP3 gene, bringing about their transcription, thereby leading to the production of pro-inflammatory cytokines/pre-cytokines. We have previously shown that Fyn contributes to LPS and TNFα-mediated NF-κB activation in microglia (Example 1; Panicker et al., 2015). To assess the role of Fyn in αSyn-mediated priming of the NLRP3 inflammasome, we first treated WT and Fyn^{-/-} microglia with aggregated αSyn for 30 min, and then prepared nuclear and cytosolic extracts. Probing the nuclear extracts revealed less αSyn-induced nuclear translocation of p65 in the Fyn^{-/-} microglial nuclear lysates relative to WT (FIG. 21A, 21B). To directly assess the role of Fyn in priming of the inflammasome, we treated WT and Fyn^{-/-} microglial cells with αSyn for 45 min. The αSyn-mediated induction of pro-IL-1β and NLRP3 mRNAs was significantly attenuated in the Fyn^{-/-} microglia. Notably, αSyn treatment did not induce NLRC4 and AIM2 inflammasome levels, demonstrating that the NLRP3 inflammasome was preferentially activated (FIG. 21C). Next, immunoblotting analysis of WT and Fyn^{-/-} lysates of microglia treated with αSyn for 2, 4 and 6 h revealed that pro-IL-1β, NLRP3 and cleaved Caspase-1 levels were significantly diminished in the Fyn^{-/-} microglia (FIG. 21D, 21E). We also knocked Fyn down using Fyn-specific siRNA and observed diminished induction of pro-IL-1β in aggregated αSyn treated microglia (FIG. 21F, 21G). Lastly, immunoblot and Luminex analyses of αSyn-treated microglial supernatants revealed diminished secretion of mature IL-1β and IL-12 from Fyn^{-/-} cells (FIG. 21H, 21I).

Fyn Contributes to αSyn Import into Microglial Cells, Thereby Contributing to NLRP3 Inflammasome-Associated Mitochondrial Dysfunction.

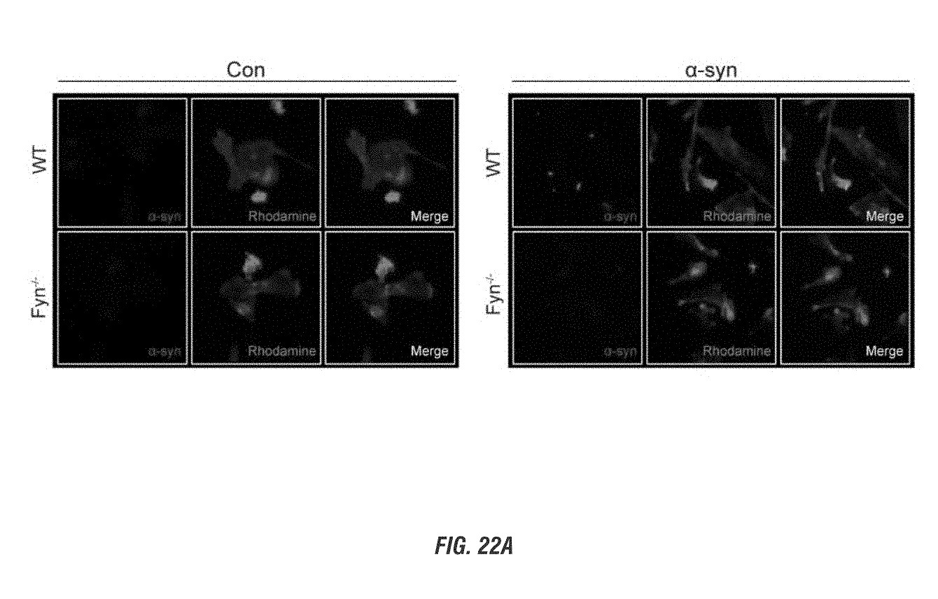

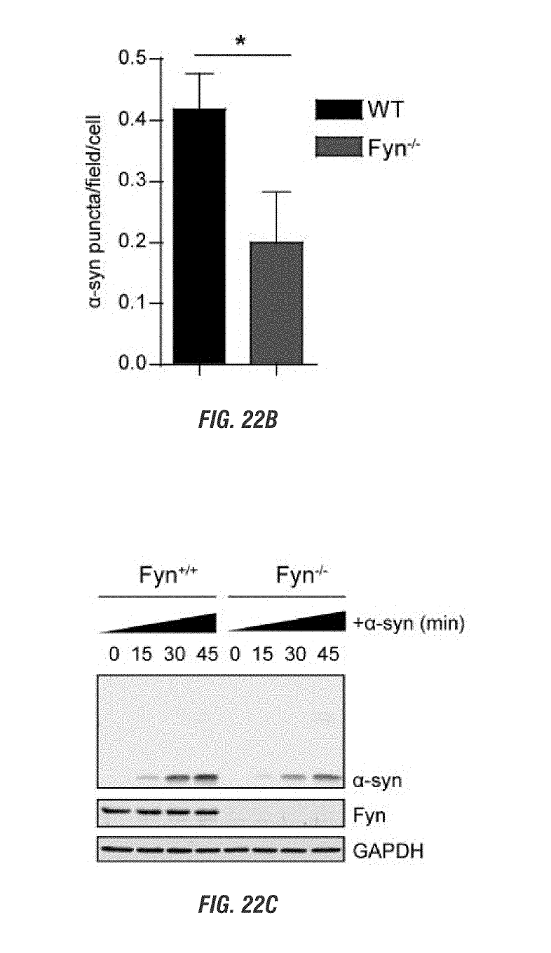

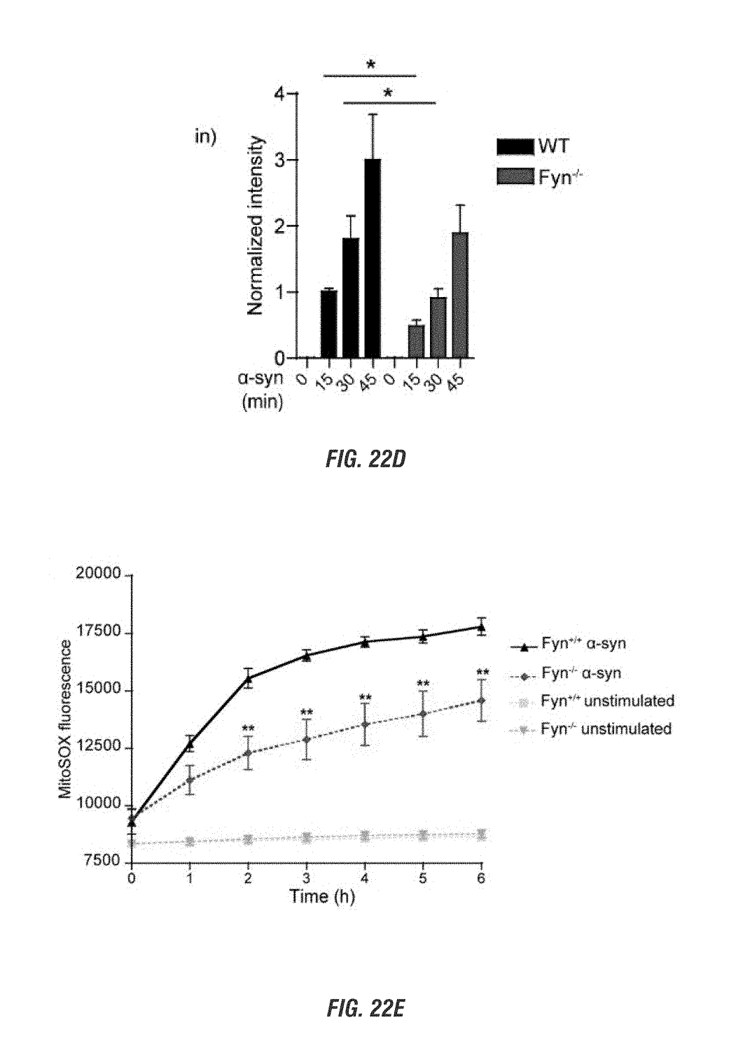

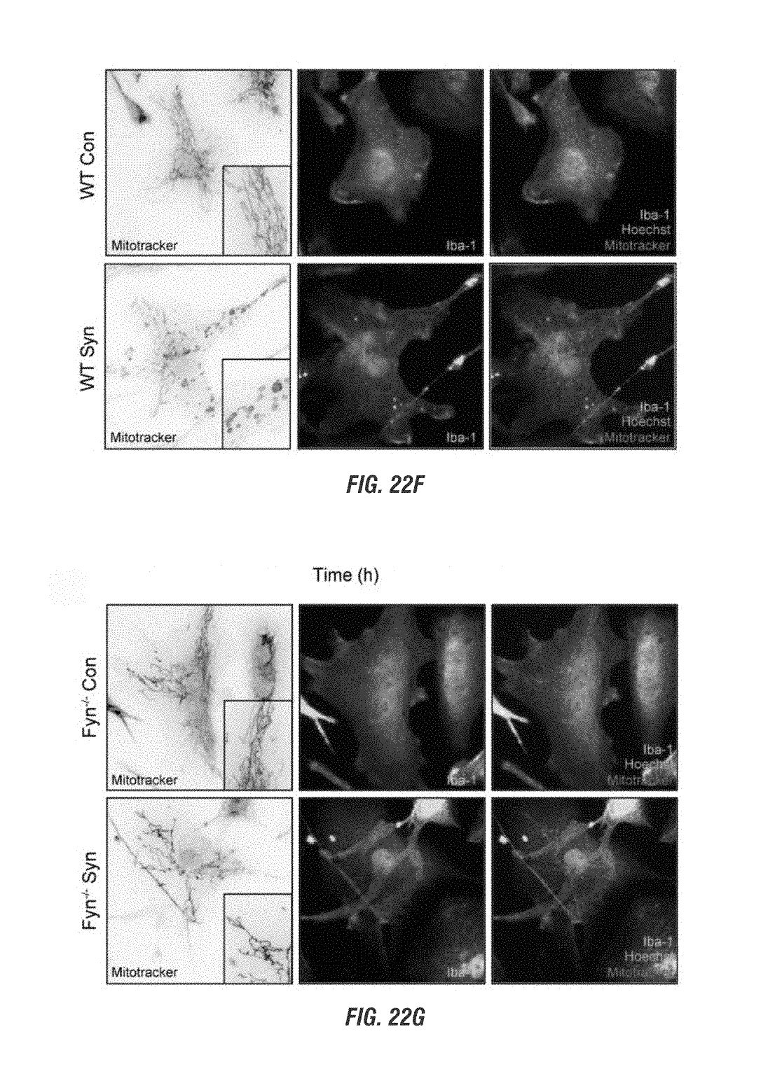

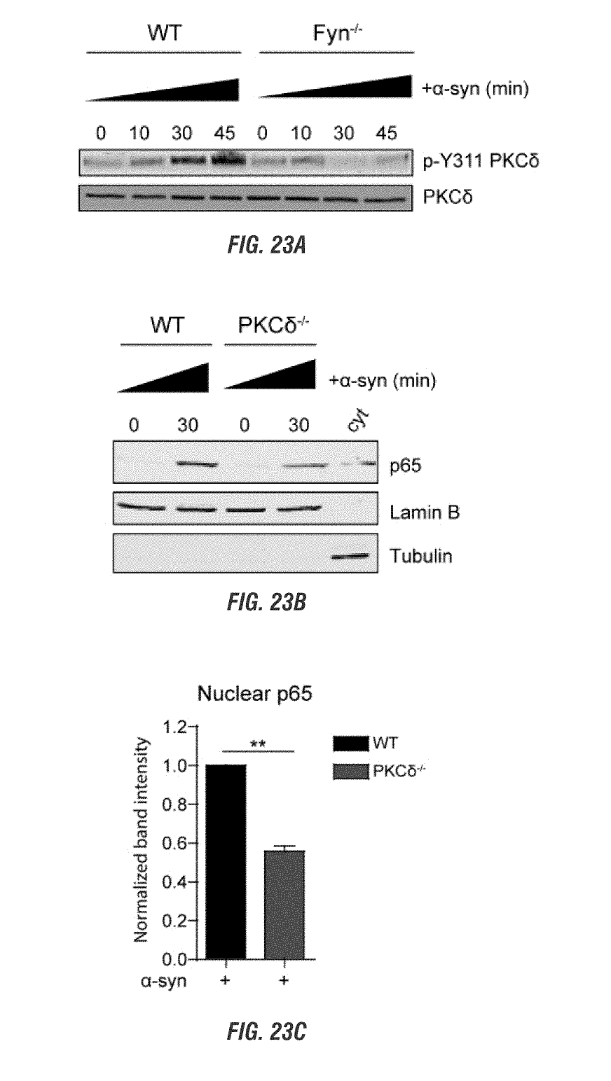

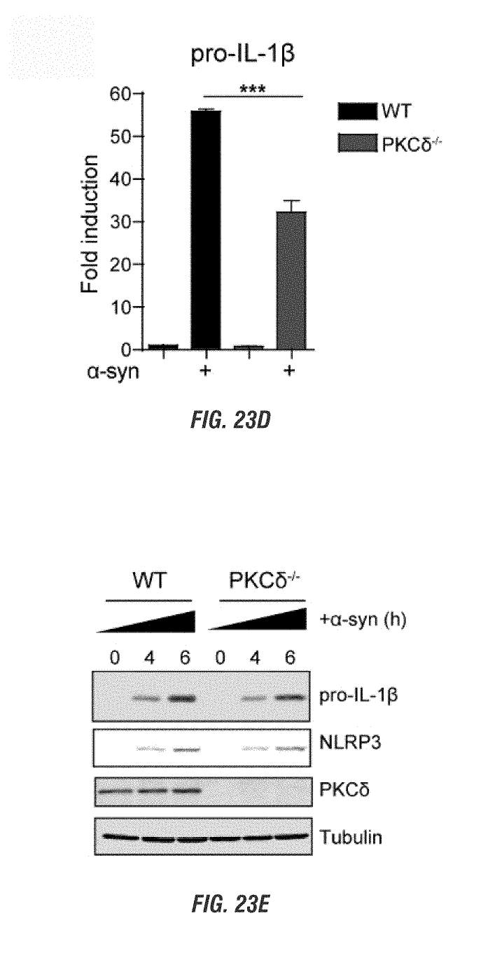

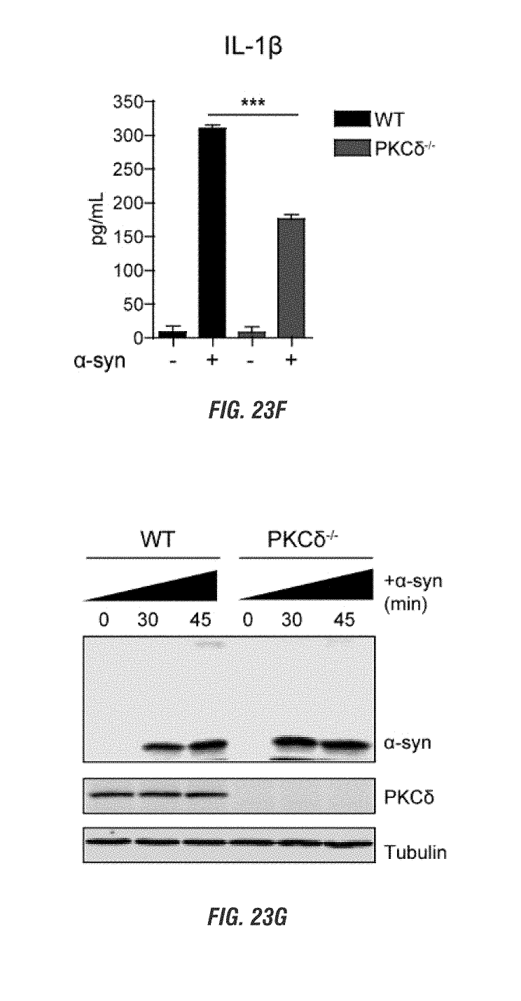

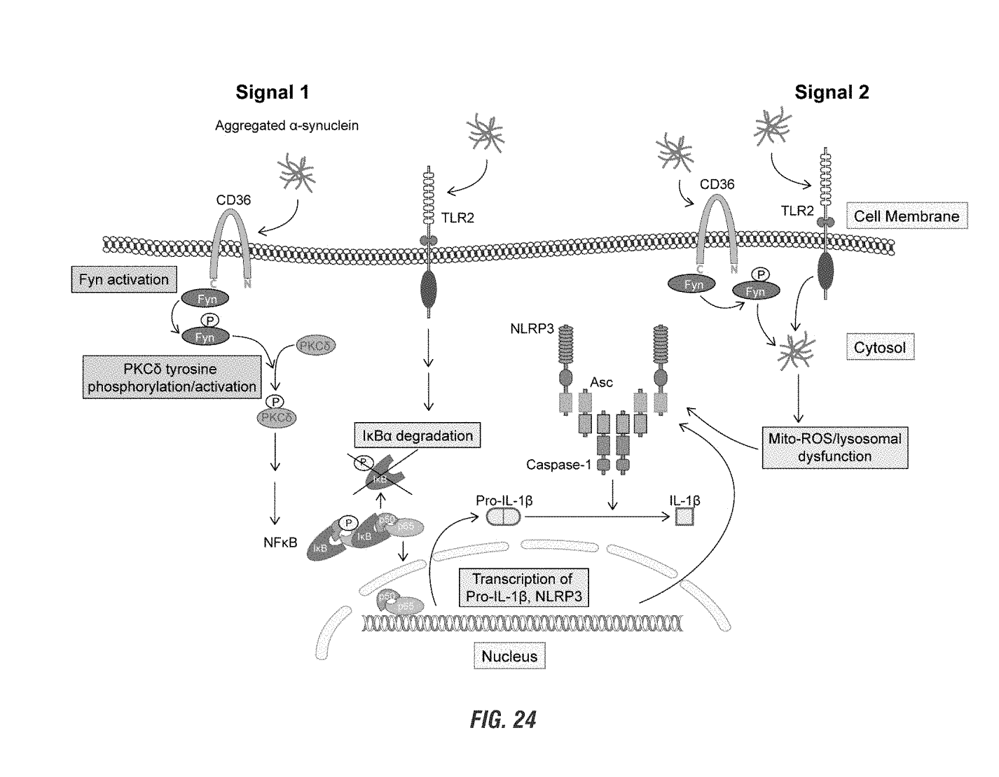

Although most existing studies agree on the steps to priming the NLRP3 inflammasome, the exact nature of the danger or activating signal remains contested. The current consensus points to lysosomal dysfunction, mitoROS generation and potassium efflux as possible molecular events leading to the assembly of the NLRP3 inflammasome. Since we observed Fyn interacting with CD36, a receptor protein involved in the uptake and aggregation seeding ability of amyloid (Sheedy et al., 2013), we tested whether Fyn plays a role in the uptake of αSyn into microglial cells by adding human αSyn to WT and Fyn^{-/-} microglia for various time points. Cells were triple washed with PBS, then fixed and stained for microglial uptake of human αSyn, which showed up as intracellular puncta. The number of puncta per cell per field were quantified. Taken together, Fyn^{-/-} microglia display diminished uptake of human αSyn (FIG. 22A, 22B). Immunoblots of whole cell lysates from WT and Fyn^{-/-} microglia treated with human αSyn for 15, 30 and 45 min indicated significantly attenuated uptake in the Fyn-deficient cells (FIG. 22C, 22D). Uptake of an inflammasome activator disrupts cellular homeostasis through several possible mechanisms. ROS generation, specifically from the mitochondria, has gained acceptance as a prime contributor to inflammasome activation (Zhou et al., 2011). We utilized the MitoSOX dye to quantify αSyn-induced mitoROS generation in WT and Fyn^{-/-} microglia. Treatment of cells with αSyn rapidly induced progressively increasing mitoROS generation to a significantly greater extent in the WT microglia than in Fyn^{-/-} microglia (FIG. 22E). Mitochondrial dysfunction is also characterized by a change in mitochondrial morphology, changing from thread-like to rounded. Microglia in our WT αSyn-treated group demonstrated rounded mitochondria 24 h post treatment, whereas the Fyn^{-/-} microglia exhibited no discernable change in mitochondrial morphology (FIG. 22F, 22G). We have previously demonstrated that upon activation following LPS and TNFα stimulation, Fyn associates with and tyrosine phosphorylates the serine threonine kinase PKCδ in microglial cells at residue Y311. We observed that αSyn also mediated an increase in p-Y311 PKCδ levels, but did not do so in the Fyn deficient microglia, suggesting a conserved pro-inflammatory signaling pathway downstream of inflammogen activation (FIG. 23A). Upon checking nuclear lysates from αSyn treated PKCδ^{+/+} and PKCδ^{-/-} cells for p65, we observed diminished activation of the NF-κB pathway, evidenced by reduced nuclear p65 in the PKCδ^{-/-} nuclear fractions, reminiscent of the Fyn-PKCδ mediated signaling cascade downstream of LPS and TNFα activation (FIG. 23B, 23C). The αSyn mediated upregulation of pro-IL-1β mRNA was also significantly attenuated in the PKCδ deficient microglia (FIG. 23D), as was the synthesis of pro-IL-1β and NLRP3 proteins (FIG. 23E) and the production of supernatant IL-1β (FIG. 23F). We also wanted to check whether PKCδ played a role in αSyn import. We used whole cell lysates from PKCδ^{+/+} and PKCδ^{-/-} microglia treated with aggregated α-synuclein for 30 and 45 minutes. Upon probing for human α-synuclein, we saw no change between either genotype with respect to αSyn import (FIG. 23G). These results suggest a bifurcation of Fyn dependent signaling, showing that Fyn activation feeds into the PKCδ dependent NF-κB pathway activation and priming of the NLRP3 inflammasome, and the PKCδ independent import of αSyn. This signaling pathway is represented in FIG. 24.

### Fyn Kinase Contributes to Microgliosis and Microglial Inflammasome Activation in the Syn-AAV Mouse Model of Parkinson's Disease

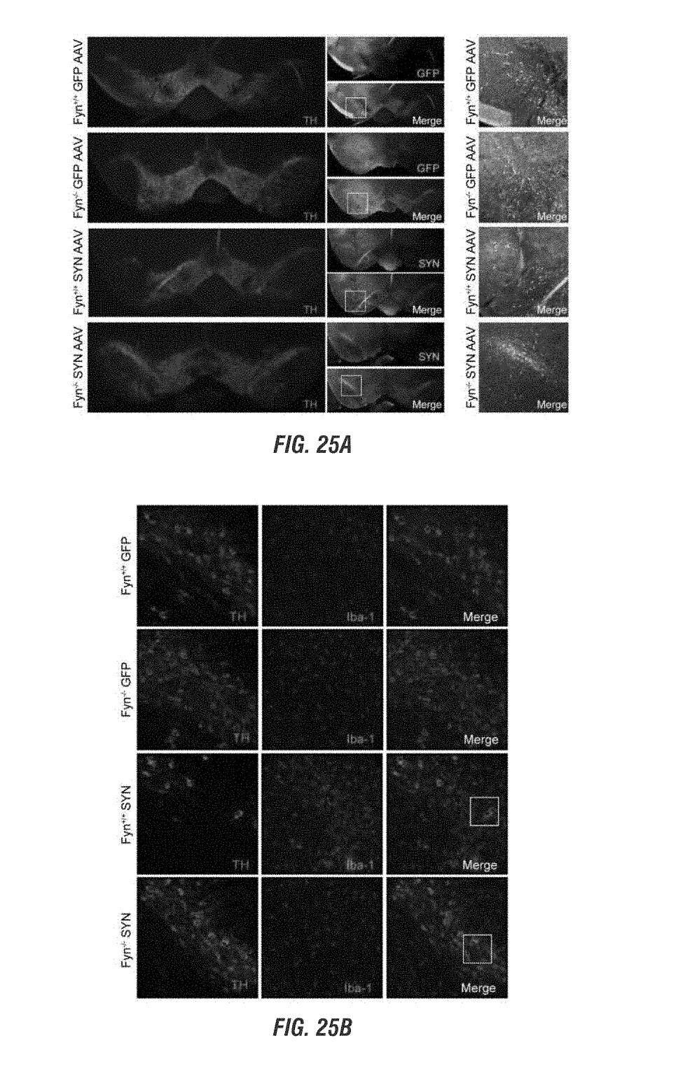

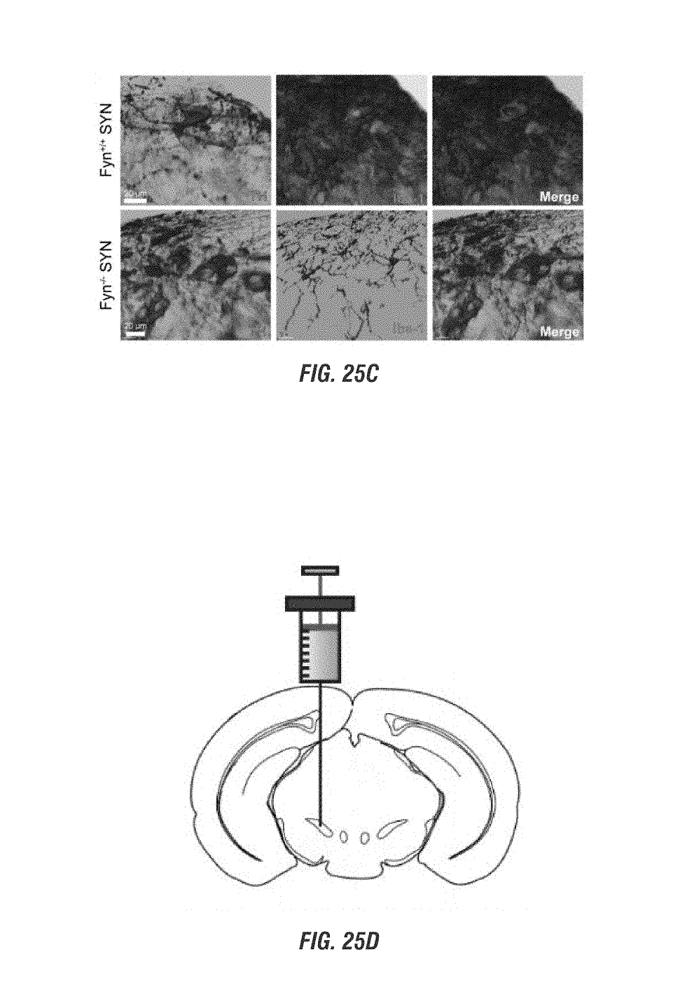

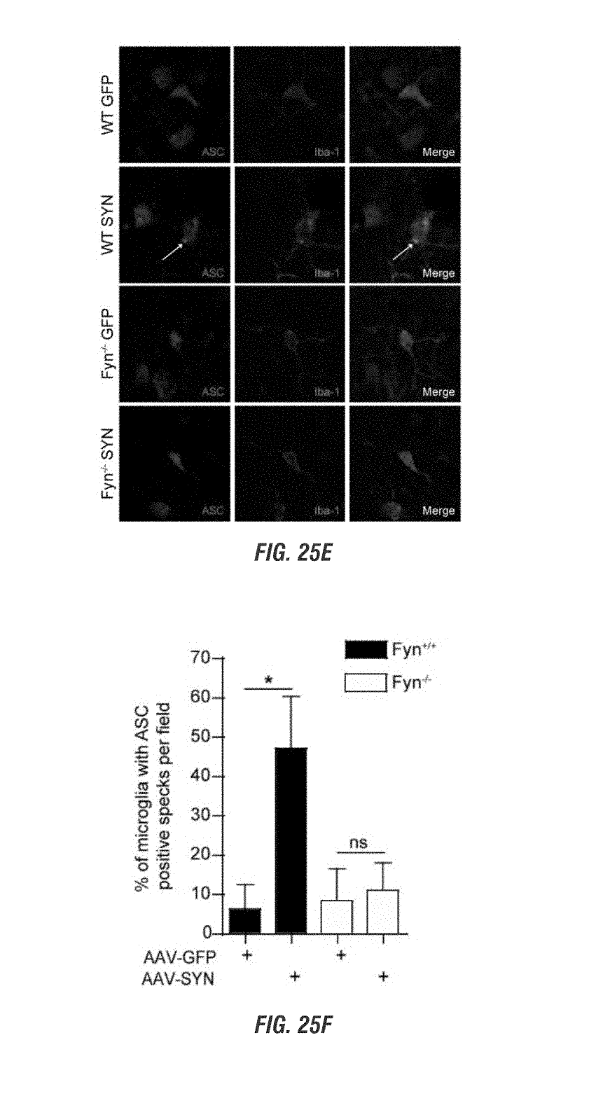

Activation of the NLRP3 inflammasome in PD models is still an active area of investigation and has not been conclusively demonstrated yet. However, there is some evidence to suggest that it may be activated under certain conditions. Subjecting the transgenic db/db diabetic cell line to a regimen of the Parkinsonian toxicant MPTP activates the NLRP3 inflammasome and exacerbates neuroinflammation and neurodegeneration (Wang et al., 2014). Moreover, αSyn AAV overexpression in the nigra significantly increases IL-1β and TNFα in a rat model of synucleinopathy (Chung et al., 2009). Viral vector-mediated overexpression of IL-1β in the mouse SN can affect dopaminergic neurodegeneration (Ferrari et al., 2006). A recent study showed that NLRP3^{-/-} mice were resistant to MPTP-induced TH-positive cell loss and dopaminergic neuron loss (Yan et al., 2015). Accordingly, to assess whether microglial inflammasome activation could be driven by αSyn overexpression in vivo we injected WT and Fyn^{-/-} mice SNs with AAVs coding for GFP and human αSyn (FIG. 25D). Brains were obtained 45 d post-injection. Successive coronal mouse brain sections were probed for the dopaminergic neuronal marker tyrosine hydroxylase (TH) and GFP or human αSyn to verify that virus injections were correctly targeted to the SNPc of the mice, as evidenced by a colocalization of the TH-positive dopaminergic neurons and the GFP or human αSyn. Interestingly, a modest loss of TH-positive cells was evident within the SNPc of WT AAV-αSyn injected mice (FIG. 25A). Strikingly, in ventral midbrain sections stained for TH and Iba-1, αSyn overexpression induced massive nigral microgliosis in the WT but not the Fyn^{-/-} mice (FIG. 25B). To visualize the degree of microgliosis with greater clarity, we used the Imaris software to construct 3D reconstructions of the Z-stack maximum projection images (FIG. 25C). One feature that validates activation of the inflammasome in vivo is the formation of ASC specks in the microglia. This was done in the APP/PS1 mouse AD model (Heneka et al., 2013). Accordingly, to assess whether microglial inflammasome activation could be driven by αSyn overexpression in vivo, we stained sections from this study with antibodies to ASC and Iba-1 and quantified the number of microglial cells per field showing ASC specks. AAV-Syn-injected WT, but not Fyn^{-/-} mice, demonstrated a significant increase in the number of ASC speck-positive microglia, showing that the AAV-Syn model affected the Fyn-dependent activation of the inflammasome in microglia (FIG. 25E, 25F).

### Saracatinib, a Src Family Kinase Inhibitor, Attenuates Aggregated α-Synuclien Induced Inflammation in Microglial Cell Line

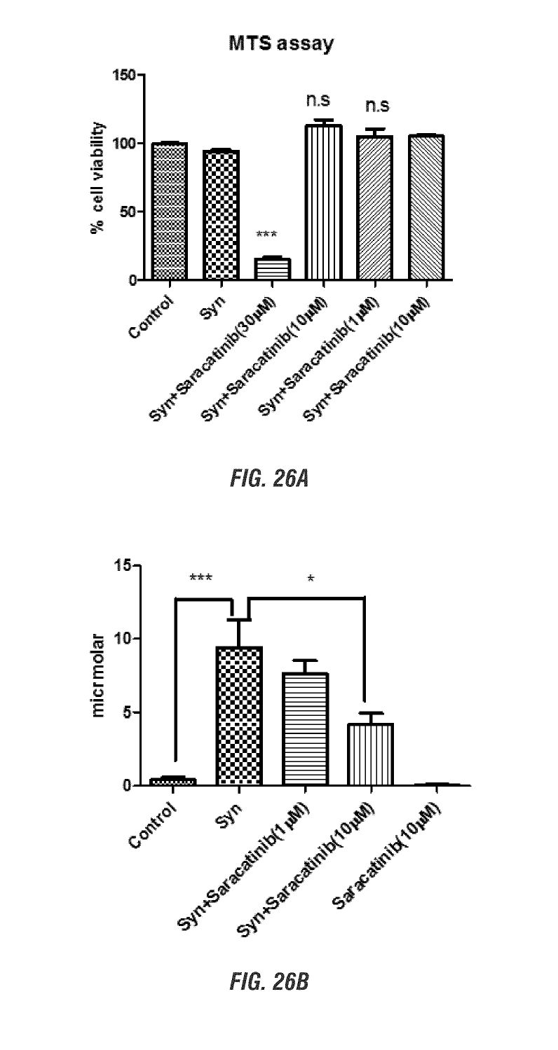

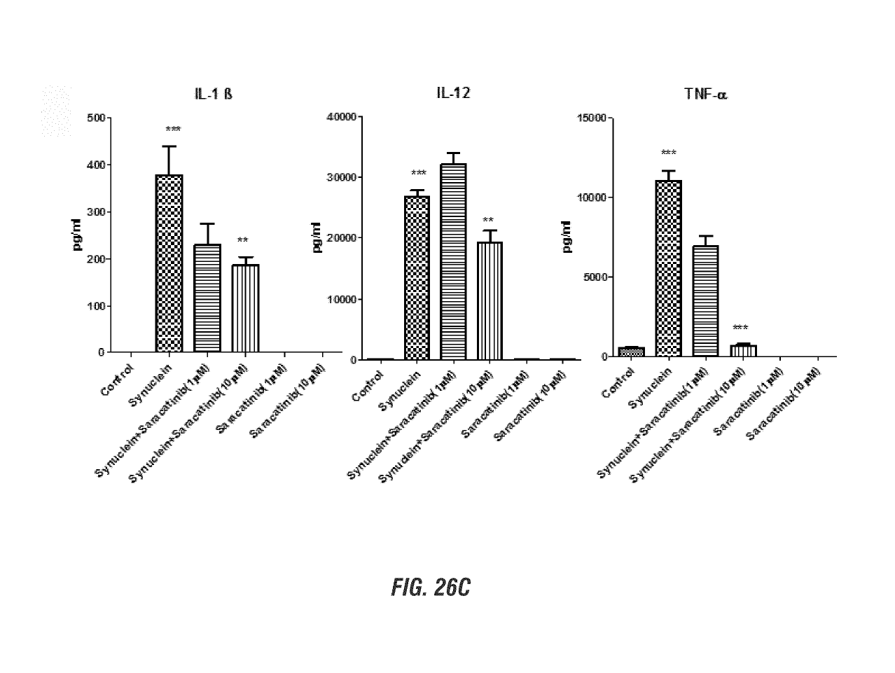

In this study microglial cell line were pretreated with 1-30 μM Saracatinib, a Fyn kinase inhibitor followed by aggregated α-synuclein (1 μM) for 24 hours in a 96 well plate. As shown in FIG. 26A saractinib did not induce cytotoxic cell death in cell viability assays. In Griess assays, saractinib significantly blocked aggregated α-synuclein induced nitrite production in a dose-dependent manner (FIG. 26B) suggesting that Fyn kinase mediates aggregated α-synuclein induced nitrite production. Further, in Luminex cytokine assays, saractinib significantly attenuated pro-inflammatory cytokines like IL-1β, 1L-12 and TNF-α production induced by treatment with aggregated α-synuclein in a dose-dependent manner (FIG. 26C). Together, these results suggest an anti-inflammatory property of saractinib.

### Sarcanatib Inhibits Microglia Activation in the SN of MPTP Animal Models of Parkinson's Disease

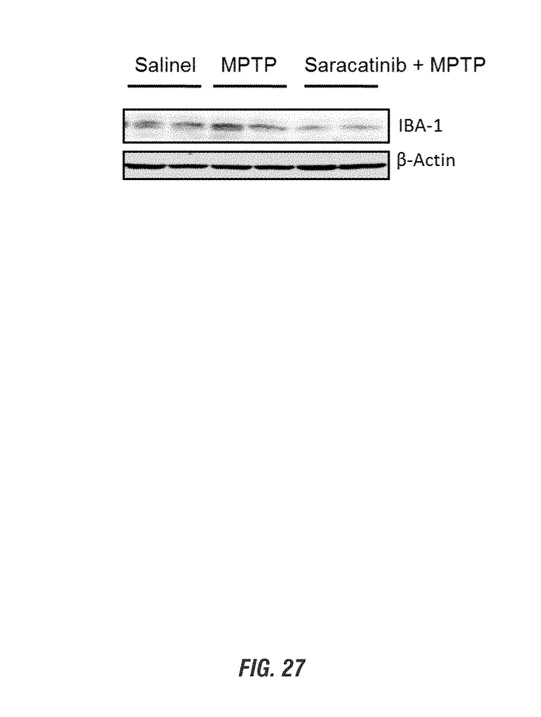

Increasing evidences suggest that activated microglial cells in the SN pars compacta (SNpc) play an important role in dopaminergic neurodegeneration in human PD patients and in the MPTP mouse model. Thus, we evaluated whether the Sarcanatib attenuates microglial activation in the substantia nigra. C57 black mice were orally administered saracatinib (5 mg/kg, once) on days 1 and 2. On day 2, after sarcanatib administration, mice were subject to acute MPTP regimen. Mice were administered 18 mg/kg MPTP (4 i.p. injections at 2 hour intervals) and control animals received saline. Mice were sacrificed 24 hrs post last MPTP injection. Brain harvested and nigral tissue lysates were subject to Western blot analysis. As shown in FIG. 27, Western blotting revealed significantly increased expression of IBA-1 in MPTP-treated SN tissues compared with saline-treated control samples, whereas orally administered sarcanatib attenuated the expression of IBA-1 in MPTP-treated mice. Together, these results strongly validate the anti-inflammatory property of saractinib in animal models and could serve as a promising therapeutic for the treatment of PD and other related neurodegenerative disorders.

### REFERENCES

1. Zhang W, Wang T, Pei Z, Miller D S, Wu X, Block M L, Wilson B, Zhou Y, Hong J S, Zhang J. Aggregated alpha-synuclein activates microglia: a process leading to disease progression in Parkinson's disease. FASEB journal: official publication of the Federation of American Societies for Experimental Biology. 2005; 19(6):533-42. Epub 2005/03/26. doi: 10.1096/fj.04-2751com. PubMed PMID: 15791003.
2. Alvarez-Erviti L, Seow Y, Schapira A H, Gardiner C, Sargent I L, Wood M J, Cooper J M. Lysosomal dysfunction increases exosome-mediated alpha-synuclein release and transmission. Neurobiology of disease. 2011; 42(3):360-7. Epub 2011/02/10. doi: 10.1016/j.nbd.2011.01.029. PubMed PMID: 21303699 PMCID: PMC3107939.
3. Emmanouilidou E, Melachroinou K, Roumeliotis T, Garbis S D, Ntzouni M, Margaritis L H, Stefanis L, Vekrellis K. Cell-produced alpha-synuclein is secreted in a calcium-dependent manner by exosomes and impacts neuronal survival. The Journal of neuroscience: the official journal of the Society for Neuroscience. 2010; 30(20):6838-51. Epub 2010/05/21. doi: 10.1523/JNEUROSCI.5699-09.2010. PubMed PMID: 20484626.
4. Pan-Montojo F, Schwarz M, Winkler C, Arnhold M, O'Sullivan G A, Pal A, Said J, Marsico G, Verbavatz J M, Rodrigo-Angulo M, Gille G, Funk R H, Reichmann H. Environmental toxins trigger PD-like progression via increased alpha-synuclein release from enteric neurons in mice. Scientific reports. 2012; 2:898. Epub 2012/12/04. doi: 10.1038/srep00898. PubMed PMID: 23205266 PMCID: PMC3510466.
5. Russo I, Bubacco L, Greggio E. Exosomes-associated neurodegeneration and progression of Parkinson's disease. American journal of neurodegenerative disease. 2012; 1(3):217-25. Epub 2013/02/06. PubMed PMID: 23383394 PMCID: PMC3560468.
6. Walsh J G, Muruve D A, Power C. Inflammasomes in the CNS. Nature reviews Neuroscience. 2014; 15(2):84-97. doi: 10.1038/nrn3638. PubMed PMID: 24399084.
7. Couch Y, Alvarez-Erviti L, Sibson N R, Wood M J, Anthony D C. The acute inflammatory response to intranigral alpha-synuclein differs significantly from intranigral lipopolysaccharide and is exacerbated by peripheral inflammation. Journal of neuroinflammation. 2011; 8:166. Epub 2011/11/30. doi: 10.1186/1742-2094-8-166. PubMed PMID: 22122884 PMCID: PMC3239418.
8. Lee H J, Suk J E, Patrick C, Bae E J, Cho J H, Rho S, Hwang D, Masliah E, Lee S J. Direct transfer of alpha-synuclein from neuron to astroglia causes inflammatory responses in synucleinopathies. The Journal of biological chemistry. 2010; 285(12):9262-72. Epub 2010/01/15. doi: 10.1074/jbc.M109.081125. PubMed PMID: 20071342 PMCID: PMC2838344.
9. Roodveldt C, Labrador-Garrido A, Gonzalez-Rey E, Fernandez-Montesinos R, Caro M, Lachaud C C, Waudby C A, Delgado M, Dobson C M, Pozo D. Glial innate immunity generated by non-aggregated alpha-synuclein in mouse: differences between wild-type and Parkinson's disease-linked mutants. PloS one. 2010; 5(10):e13481. Epub 2010/11/05. doi: 10.1371/journal.pone.0013481. PubMed PMID: 21048992 PMCID: PMC2964342.
10. Alvarez-Erviti L, Couch Y, Richardson J, Cooper J M, Wood M J. Alpha-synuclein release by neurons activates the inflammatory response in a microglial cell line. Neuroscience research. 2011; 69(4):337-42. Epub 2011/01/25. doi: 10.1016/j.neures.2010.12.020. PubMed PMID: 21255620.
11. Desplats P, Lee H J, Bae E J, Patrick C, Rockenstein E, Crews L, Spencer B, Masliah E, Lee S J. Inclusion formation and neuronal cell death through neuron-to-neuron transmission of alpha-synuclein. Proceedings of the National Academy of Sciences of the United States of America. 2009; 106(31):13010-5. Epub 2009/08/05. doi: 10.1073/pnas.0903691106. PubMed PMID: 19651612 PMCID: PMC2722313.
12. Lee H J, Patel S, Lee S J. Intravesicular localization and exocytosis of alpha-synuclein and its aggregates. The Journal of neuroscience: the official journal of the Society for Neuroscience. 2005; 25(25):6016-24. Epub 2005/06/25. doi: 10.1523/JNEUROSCI.0692-05.2005. PubMed PMID: 15976091.
13. Lee H J, Baek S M, Ho D H, Suk J E, Cho E D, Lee S J. Dopamine promotes formation and secretion of non-fibrillar alpha-synuclein oligomers. Experimental & molecular medicine. 2011; 43(4):216-22. Epub 2011/03/19. doi: 10.3858/emm.2011.43.4.026. PubMed PMID: 21415592 PMCID: PMC3085740.
14. Jang A, Lee H J, Suk J E, Jung J W, Kim K P, Lee S J. Non-classical exocytosis of alpha-synuclein is sensitive to folding states and promoted under stress conditions. Journal of neurochemistry. 2010; 113(5):1263-74. Epub 2010/03/30. doi: 10.1111/j.1471-4159.2010.06695.x. PubMed PMID: 20345754.
15. Vella L J, Sharples R A, Nisbet R M, Cappai R, Hill A F. The role of exosomes in the processing of proteins associated with neurodegenerative diseases. European biophysics journal: EBJ. 2008; 37(3):323-32. Epub 2007/12/08. doi: 10.1007/s00249-007-0246-z. PubMed PMID: 18064447.
16. Kim C, Ho D H, Suk J E, You S, Michael S, Kang J, Joong Lee S, Masliah E, Hwang D, Lee H J, Lee S J. Neuron-released oligomeric alpha-synuclein is an endogenous agonist of TLR2 for paracrine activation of microglia. Nature communications. 2013; 4:1562. Epub 2013/03/07. doi: 10.1038/ncomms2534. PubMed PMID: 23463005.
17. Su X, Maguire-Zeiss K A, Giuliano R, Prifti L, Venkatesh K, Federoff H J. Synuclein activates microglia in a model of Parkinson's disease. Neurobiology of aging. 2008; 29(11):1690-701. Epub 2007/06/01. doi: 10.1016/j.neurobiolaging.2007.04.006. PubMed PMID: 17537546 PMCID: PMC2621109.
18. Franchi L, Munoz-Planillo R, Nunez G. Sensing and reacting to microbes through the inflammasomes. Nature immunology. 2012; 13(4):325-32. doi: 10.1038/ni.2231. PubMed PMID: 22430785 PMCID: PMC3449002.
19. Liu L, Chan C. The role of inflammasome in Alzheimer's disease. Ageing research reviews. 2014; 15C:6-15. doi: 10.1016/j.arr.2013.12.007. PubMed PMID: 24561250 PMCID: PMC4029867.
20. Beraud D, Twomey M, Bloom B, Mittereder A, Ton V, Neitzke K, Chasovskikh S, Mhyre T R, Maguire-Zeiss K A. alpha-Synuclein Alters Toll-Like Receptor Expression. Frontiers in neuroscience. 2011; 5:80. Epub 2011/07/13. doi: 10.3389/fnins.2011.00080. PubMed PMID: 21747756 PMCID: PMC3128248.
21. Su X, Federoff H J, Maguire-Zeiss K A. Mutant alpha-synuclein overexpression mediates early proinflammatory activity. Neurotoxicity research. 2009; 16(3):238-54. Epub 2009/06/16. doi: 10.1007/s12640-009-9053-x. PubMed PMID: 19526281 PMCID: PMC2877375.
22. Marques O, Outeiro T F. Alpha-synuclein: from secretion to dysfunction and death. Cell death & disease. 2012; 3:e350. Epub 2012/07/25. doi: 10.1038/cddis.2012.94. PubMed PMID: 22825468 PMCID: PMC3406593.
23. Kettenmann H, Hanisch U K, Noda M, Verkhratsky A. Physiology of microglia. Physiological reviews. 2011; 91(2):461-553. Epub 2011/04/30. doi: 10.1152/physrev.00011.2010. PubMed PMID: 215277

1: A method of treating a subject in need thereof for Parkinson's disease comprising:
administering to said subject an effective amount of saracatinib, or a pharmaceutically acceptable salt, prodrug or solvate thereof, so that Fyn tyrosine kinase activity is inhibited, and wherein said Parkinson's disease does not comprise amyloid-beta accumulation.

2: The method of claim 1, wherein the saracatinib is administered intravenously, intramuscularly, subcutaneously, intraorbitally, intracapsularly, intraperitoneally, intrarectally, or intracisternally to said subject.

3: The method of claim 8, wherein said administration is orally.

4: The method of claim 1 wherein said subject is a human.

5: The method of claim 1 wherein said saracatinib is selected from the group consisting of saracatinib free base, saracatinib difumarate, and a combination thereof.

6: The method of claim 1 wherein said saracatinib is saracatinib free base.

7: The method of claim 1 wherein said saracatinib is saracatinib difumarate.

8: The method of claim 1 wherein said effective amount of saracatinib includes an amount sufficient for a cerebral spinal fluid trough concentration from about 0.9 nM to about 2.2 nM.

9: The method of claim 1 wherein said saracatinib administration includes a pharmaceutically acceptable carrier.

10: The method of claim 1 further comprising the step of administering a second Parkinson's disease treating compound.

11: The method of claim 10 wherein said second Parkinson's treating compound is a second Src tyrosine kinase inhibitor.

12: The method of claim 10 wherein said second Parkinson's treating compound is one or more of the following: a dopamine agonists, amantadine, an anticholinergic, a COMT inhibitor, or a MAO-B inhibitor.

13: A method of treating neuroinflammation in a central nervous system of a mammal in need thereof, said method comprising:
administering to said mammal a Fyn tyrosine kinase inhibitor, thereby decreasing inflammation, and wherein said neuroinflammation is not associated with amyloid-beta accumulation.

14: The method of claim 13, wherein said Fyn tyrosine kinase inhibitor is a polynucleotide, peptide, polysaccharide, lipid, small molecule or drug.

15: The method of claim 13, wherein said Fyn tyrosine kinase inhibitor is N-(5-chloro-1,3-benzodioxol-4-yl)-7-[2-(4-methylpiperazin-1-yl)ethoxy]-5-(oxan-4-yloxy)quinazolin-4-amine.

16: The method of claim 13, wherein the neuroinflammation is associated with Parkinson's disease, Tourette's syndrome, schizophrenia, Huntington's disease, symptoms of attention deficit hyperactivity disorder, drug abuse or clinical depression.

17: The method of claim 13 wherein the mammal is suffering from Parkinson's disease.

18: The method of claim 13, wherein the Fyn tyrosine kinase inhibitor is administered intravenously, intramuscularly, subcutaneously, intraorbitally, intracapsularly, intraperitoneally, intrarectally, orally, or intracisternally to the mammal.

19: The method of claim 13, wherein said Fyn tyrosine kinase inhibitor is administered to said mammal subsequent, prior or during the onset of Parkinson's disease.

20: The method of claim 13 wherein said Fyn tyrosine kinase inhibitor is administered with a second Parkinson's disease treating agent.

21: A method for identifying a potential therapeutic agent for treating Parkinson's disease or other diseases associated with neuroinflammation comprising:
Contacting a test compound with a cell, and
assaying for the ability of said compound to inhibit Fyn tyrosine kinase activity or expression.

22: The method of claim 7 wherein the cell is a mammalian cell.

23: A pharmaceutical composition for treating an animal with Parkinson's disease comprising:
a first Parkinson's treating compound of saracatinib;
a second Parkinson's treating compound; and
a pharmaceutically acceptable carrier.

24: The pharmaceutical composition of claim 24 wherein said saracatinib is selected from the group consisting of saracatinib free base, saracatinib difumarate, and/or a combination thereof.

25: The pharmaceutical composition of claim 24 wherein said saracatinib is saracatinib free base.

26: The pharmaceutical composition of claim 24 wherein said saracatinib is saracatinib difumarate.

27: The pharmaceutical composition of claim 24 The method of claim 1 wherein said saracatinib is N-(5-chloro-1,3-benzodioxol-4-yl)-7-[2-(4-methylpiperazin-1-yl)ethoxy]-5-(oxan-4-yloxy)quinazolin-4-amine or a salt or derivative thereof.

28: The pharmaceutical composition of claim 24 wherein said second Parkinson's treating compound is a second Src tyrosine kinase inhibitor.

29: The pharmaceutical composition of claim 29 wherein the Src tyrosine kinase inhibitor is an Fyn tyrosine kinase inhibitor.

30: The pharmaceutical composition of claim 24 wherein said second Parkinson's treating compound is one or more of the following: a dopamine agonist, amantadine, an anticholinergic, a COMT Inhibitor, or a MAO-B inhibitor.
